(19) United States
(12) Patent Application Publication
(10) Pub. No.: US 2021/0338652 A1
(43) Pub. Date: Nov. 4, 2021

(54) COMPOSITION AND USE FOR THE TREATMENT OF PARKINSON'S DISEASE AND RELATED DISORDERS

(71) Applicant: CHASE THERAPEUTICS CORPORATION, Washington, DC (US)

(72) Inventors: Thomas N. CHASE, Washington, DC (US); Kathleen E. CLARENCE-SMITH, Washington, DC (US)

(73) Assignee: CHASE THERAPEUTICS CORPORATION, Washington, DC (US)

(21) Appl. No.: 17/279,644

(22) PCT Filed: Sep. 24, 2019
(86) PCT No.: PCT/US2019/052705

§ 371 Date: Mar. 25, 2021

Related U.S. Application Data
(60) Provisional application No. 62/735,997, filed on Sep. 25, 2018.
(60) Provisional application No. 62/785,602, filed on Dec. 27, 2018.
(60) Provisional application No. 62/785,605, filed on Dec. 27, 2018.
(60) Provisional application No. 62/785,606, filed on Dec. 27, 2018.
(60) Provisional application No. 62/787,614, filed on Jan. 2, 2019.
(60) Provisional application No. 62/817,274, filed on Mar. 12, 2019.
(60) Provisional application No. 62/840,539, filed on Apr. 30, 2019.

(51) Int. Cl. A61K 31/454; A61P 1/08; A61K 31/428

(52) U.S. Cl. CPC A61K 31/454; A61K 31/428; A61P 1/08

(57) ABSTRACT

The present invention provides a pharmaceutical combination comprising an inhibitor of dopamine agonist adverse effects and a dopamine agonist, for treating Parkinson's disease and Parkinson's disease-related disorders.

### RELATED APPLICATIONS

This application claims the benefit of U.S. Provisional Patent Application Ser. No. 62/735,997, filed Sep. 25, 2018, and U.S. Provisional Patent Application Ser. No. 62/785,602 filed on Dec. 27, 2018, and U.S. Provisional Patent Application Ser. No. 62/785,605, filed Dec. 27, 2018, U.S. Provisional Patent Application Ser. No. 62/785,606, filed Dec. 27, 2018, U.S. Provisional Patent Application Ser. No. 62/787,614, filed Jan. 2, 2019, and U.S. Provisional Patent Application Ser. No. 62/817,274 filed on Mar. 12, 2018, and U.S. Provisional Patent Application Ser. No. 62/840,539, filed Apr. 30, 2019, the disclosures of which are incorporated herein in their entirety by reference.

### FIELD OF THE INVENTION

The present invention pertains to the field of the treatment of Parkinson's disease and related disorders, which benefit from drugs that augment dopamine mediated neurotransmission in the central nervous system.

### OBJECT OF THE INVENTION

The present invention concerns an inhibitor of the adverse effects (or events) of dopaminergic agents (“dopamine-agonist Adverse Effects Inhibitor”, herein below simply referred to as “AEsI”) in combination with a pharmaceutical agent of the DA-agonist class (“DA-agonist”), for use in the treatment of Parkinson's disease and related disorders where stimulation of dopaminergic transmission in the central nervous system (“CNS”) provides benefits.

### Definitions

“CNS”: Central Nervous System.

“AD”: Alzheimer's Disease.

“PD”: Parkinson's disease.

“RLS”: Restless Leg Syndrome.

“PMND”: Protein Misfolding Neurodegenerative Disease (or Disorder).

“LD”: levodopa.

“Disease selected from the group consisting of PD and PD-related disorders”: refers to Parkinson's disease and PD-related disorders including RLS and motor symptoms in some patients suffering from atypical or Parkinson plus syndromes such as multiple system atrophy (“MSA”), Dementia with Lewy Bodies (“DLB”), Lewy Body Dementia (“LBD”), Dopamine-responsive Dystonia Syndrome (“DDS”), Progressive Supranuclear Palsy (“PSP”), Fronto-Temporal Lobe Dementia (“FTLD”) and Corticobasal Degeneration (“CBD”).

“IR”: Immediate Release of the active ingredient from a composition.

“ER”: Extended Release of the active ingredient from a composition.

“GI”: Gastro-Intestinal.

“TDDS”: Transdermal Drug Delivery System.

“DA-agonist(s)”: dopamine receptor agonist(s).

“Dopaminergic”: involving DA as a neurotransmitter; drugs with this effect are used to treat PD and PD-related disorders.

“Effective daily dose of a DA-agonist” the daily dose of a DA-agonist shown effective or approved for the treatment of PD or of hyperprolactinemia.

“Effective dose per unit form of a DA-agonist” the dose of a DA-agonist per unit form shown effective or approved for the treatment of PD or of hyperprolactinemia when given one or more times per day.

“Peripheral DA-antagonist”; in the context of the present disclosure, a substance that blocks dopamine peripheral receptors thus acting as anti-constipation, and as an antiemetic used in the treatment of nausea and vomiting.

“AEs”: Adverse Effects (or Events): in the context of the present disclosure, the adverse effects (or events) typical of dopaminergic agents, in particular nausea, vomiting, diaphoresis, constipation, lightheadedness and headache.

“AEsI” (Adverse Effects—or Event—Inhibitor): Inhibitor of the Adverse Effects of DA-agonists, also referred to as “DA-agonist-AEsI”; in the context of the present disclosure, a substance that inhibits the above pramipexole AEs, in particular a peripheral DA-antagonist, a 5HT3-antagonist or a NK1-antagonist.

“Effective daily dose of an AEsI”: the daily dose of an AEsI as least as high as that shown effective or approved for preventing or treating constipation, nausea and vomiting in particular in pediatric or adult patients undergoing cancer chemotherapy according to the current protocols for said treatment or in patients suffering from motion sickness or dizziness. In the context of the present invention, said effective daily dose is from 1 μg to 600 mg.

“Effective dose per unit form of an AEsI”: the dose of an AEsI per unit form as least as high as that shown effective or approved for the prevention or treatment of constipation, nausea or vomiting of any nature, in particular in the case of post-operative nausea and vomiting, in pediatric or adult patients undergoing cancer chemotherapy according to the current protocols for said treatment or in patients suffering from motion sickness or dizziness. In the context of the present invention, said effective dose per unit form is from 1 μg to 600 mg.

“Pramipexole”: International Non-proprietary name of (S)-6-propylamino-4,5,6,7-tetrahydro-1,3-benzothiazole-2-amine Unless otherwise specified “pramipexole” include the free bases and pharmaceutically acceptable salts thereof; and the relative doses (daily or per unit form) are given in equivalents of pramipexole dihydrochloride monohydrate.

“Salts or solvates thereof”, “salt or solvate thereof”, and “salts and solvates thereof”: these expressions, in reference to a cited DA-antagonists, AEsI or of DA-agonist, in particular to domperidone and pramipexole, indicates that the salt of any of said cited DA-antagonists, AEsIs or DA-agonists, in particular domperidone and pramipexole, may be solvated with a solvent, normally water.

The term “comprise,” “comprises,” “comprising” “include,” “includes,” and “including” are interchangeable and not intended to be limiting. It is to be further understood that where descriptions of various embodiments use the term “comprising,” those skilled in the art would understand that the present disclosure also contemplates such embodiments alternatively described using the language “consisting essentially of” or “consisting of”.

### BACKGROUND OF THE INVENTION

Dopamine receptor agonists are pharmaceutical compounds that stimulate dopamine receptors. As a result, they activate intraneuronal signaling pathways through trimeric G-proteins and β-arrestins, ultimately leading to characteristic changes in gene transcription and nerve impulse activity. Drugs in this class are a widely used in the palliative treatment of Parkinson's disease (PD) and related disorders.

Neurodegenerative disorders cause a progressive injury and death of nerve cells in the central nervous system. They include Parkinson's disease and related disorders. All are incurable, resulting in an inexorable loss of neurologic and psychiatric function. Although the clinical features of these disorders differ, they all share one similar characteristic—pathogenesis involves a prion-like misprocessing of a normal brain protein into an aggregated form (misfolded protein) that is capable of replication, propagation and neurotoxicity. In PD for example the misfolding protein is alpha-synuclein.

As research progresses, many similarities appear that relate these protein misfolding neurodegenerative diseases to one another on a cellular and sub-cellular level. Discovering these similarities offers hope for therapeutic advances that might help could ameliorate many diseases simultaneously. There are many parallels between different neurodegenerative disorders including atypical protein assemblies as well as induced cell death. Neurodegeneration can be found in many different levels of neuronal circuitry ranging from molecular to systemic.

The synthesis and misfolding of oligomeric/aggregated species of such proteins as amyloid-β, tau and alpha-synuclein now appear to be the predominant pathology underlying most, if not all, neurodegenerative disorders, including AD as well as Parkinson's disease and related disorders. Whilst end stage insoluble products of aggregation have been well characterized in human and animal models of disease, increasing evidence from in vitro and in vivo studies indicates that soluble intermediates of aggregation, i.e. oligomers, especially those in the 4 to 20 mers range, may be the key species that mediate toxicity and underlie seeding and spreading in disease (Choi and Gandhi 2018).

A-β is a 38 to 43 amino acid peptide generated by the sequential proteolytic cleavage of amyloid precursor protein (“APP”) by β- and γ-secretases (Chow et al. 2010). It is thought that the over-production of A-β generated from APP plays a role in AD development. Soluble A-β oligomers, on the other hand, have been shown to produce cognitive deficits in the absence of plaques (Gandy et al. 2010). The larger aggregates are not essential to cognitive impairment (Petersen et al. 2013) nor responsible for neurodegeneration and the smaller soluble oligomers are presumed to be the toxic species of A-β. The toxic soluble oligomers are spherical in shape ranging from about 3 to 10 nm. These spheroidal structures come together forming strings of beads, termed protofibrils, which reportedly also possess toxic effects (Glabe 2006).

Alpha-synuclein, a protein composed of 140 amino acids encoded by the SNCA (Synuclein-Alpha) gene, is abundantly expressed in the human brain and to a lesser extent in various other organs. In brain, alpha-synuclein (hereafter also referred to as simply “synuclein”) is mainly found in neuronal terminals, especially in the cortex, hippocampus, substantia nigra and cerebellum, where it contributes to the regulation of neurotransmitter release, and passes into the peripheral blood stream (Marques and Outeiro, 2012), in part packaged within exosomal vesicles originating from the CNS (Shi et al. 2014).

Many amyloidogenic proteins, such as tau and amyloid-beta (A-β) species, may also be aberrantly converted from their normal monomeric form into soluble intermediates of aggregation, i.e., oligomeric species that can become neurotoxic. For A-β, toxic oligomers are now thought to range from 8-24 mers while α-synuclein oligomers are 6-18 mers and tau oligomers are 3-15 mers (Sengupta et al 2016), suggesting a universal mechanism of toxicity for amyloid proteins such as tau (Gerson and Kayed, 2013, cited in Sengupta et al. 2016), α-Synuclein (Sengupta et al. 2016), and TAR DNA-binding protein 43 (TDP-43) (Choksi et al. 2014, Fang et al. 2014, both cited in Sengupta et al. 2016).

Under normal circumstances, these proteins appear to form stably folded oligomers that resist aggregation. But, in certain pathological conditions, for unknown reasons, they misfold, oligomerize and aggregate (with the eventual formation of fibrils). Somewhere along this aberrant pathway, toxic misfolded protein species are believed to be formed which may also pass into the peripheral (systemic) circulation.

Aberrant protein oligomerization and aggregation are now thought to be the cause of PMNDs, notably PD, LBD, DLB, Parkinsonian disorders associated with glucocerebrosidase (GBA) mutations, MSA, AD, HD, multiple tauopathies, and several other disorders, which are collectively referred to as “synucleinopathies”. Alpha-synuclein is a ubiquitous protein that is especially abundant in the brain and has been postulated to play a central role in the pathogenesis of Parkinson's disease (PD), some cases of Alzheimer's disease, and other neurodegenerative disorders (Sweeney et al 2017).

Synucleinopathies are generally defined as a group of protein misfolding neurodegenerative disorders characterized in part by the intracellular accumulation of abnormal synuclein aggregates, some of which are toxic and contribute to the pathogenesis of the aforementioned disorders.

An abnormal ratio of monomeric to oligomeric synuclein species, or more specifically of particular oligomeric species, in brain-derived exosomes in peripheral blood of a patient, is postulated to be a diagnostic hallmark of a synucleinopathy and thus, for example, of one of the aforementioned neurodegenerative disorders of the human CNS. PD is a common neurodegenerative disorder of the human CNS (Poewe et al. 2017) typically presenting with three major clinical signs: resting tremor, bradykinesia, and muscular rigidity. In addition, postural instability and various neurobehavioral disabilities may occur. In the US alone it is now estimated that well over one million individuals are afflicted by this inexorably progressive disorder. Along with the aging of the American population, prevalence rates and societal costs are expected to rise exponentially. Parkinsonian signs largely reflect a loss of dopamine-containing neurons in the basal ganglia. Drugs now used to relieve symptoms generally act by restoring brain dopaminergic function (Connolly and Lang. 2014). None are known to alter the basic parkinsonian disease process. Indeed, notwithstanding prodigious investigative efforts over the past half-century, the cause and cure of these fatal disorders remain elusive.

Mutations in the glucocerebrosidase gene (GBA) can result in the autosomal recessive disorder Gaucher disease, Different lines of evidence suggest that mutant GBA can be a risk factor for some cases of Parkinson's disease. Indeed, GBA mutations are now thought to be the single largest risk factor for development of idiopathic PD. Clinically, on imaging and pharmacologically, GBA PD is nearly identical to idiopathic PD (O'Regan et al. 2017). The molecular mechanisms which lead to this increased PD risk in GBA mutation carriers are not fully elucidated, but have been shown to be associated with the accumulation of synuclein (Soria et al. 2017).

Other disorders, which may involve a decrement in dopaminergic function that improves with levodopa treatment, include RLS, dopamine responsive dystonia as well as possibly such disorders as dementia with Lewy bodies (DLB), Lewy body dementia (LBD), progressive supranuclear palsy, corticobasal degeneration (CBD) and multiple systems atrophy.

Multiple system atrophy (“MSA”) is a neurodegenerative disease of undetermined etiology that presents with parkinsonism along with signs of autonomic and other motor system dysfunction. A mild to moderate striatal loss of DA is a feature of some forms of this disorder. A reduction in striatal dopamine D2 receptor binding has also been reported, possibly contributing to the limited response to levodopa even in those with predominantly parkinsonian features. MSA with orthostatic hypotension is the current term for a neurological disorder that was once called Shy-Drager syndrome. A progressive disorder of the central and autonomic nervous systems, it is characterized by orthostatic hypotension (an excessive drop in blood pressure when standing up), which causes dizziness and fainting. Multiple system atrophy can occur without orthostatic hypotension, but instead have urinary tract involvement (urgency/incontinence). Neurologists classify the disorder into 3 types: the Parkinsonian-type includes symptoms of Parkinson's disease such as slow movement, stiff muscles, and tremor; the cerebellar-type, which causes problems with coordination and speech; and the combined-type, which includes symptoms of both parkinsonism and cerebellar dysfunction. Problems with urinary incontinence, constipation, and sexual impotence may happen early in the course of the disease. Other symptoms include generalized weakness, double vision or other vision disturbances, difficulty breathing and swallowing, sleep disturbances, and decreased sweating. Because the disease resembles others, a correct diagnosis may take years.

Alzheimer disease (AD) is characterized by deposition of β-amyloid peptides, phosphorylated tau protein (3- and 4-repeat tau) and frequent α-synuclein (aSyn, as abbreviated by the authors) deposits (Jellinger 2008). Lewy body diseases, such as sporadic Parkinson disease (PD) and dementia with Lewy bodies (DLB), show a-Synuclein-positive deposits in neurons, neurites, glia, and presynaptic terminals, while frontotemporal dementias present tau-positive and tau-negative, ubiquitin, and TDP-43-positive neuronal and glial inclusions (Jellinger 2008b). Molecular interactions between major proteins, which may occur within the same brain in various distribution patterns, cause variable phenotypes and mixed pathologies, e.g. AD with a-Synuclein pathology in the brainstem and amygdala, PD and DLB with AD lesions, and frontotemporal dementia with a mixture of various deposits, while others are featured by one principal pathology without other lesions (e.g. tangle-predominant type of dementia, pure PD, brainstem-predominant LBD) (Jellinger 2008b). In Alzheimer's disease, amyloid-β and tau proteins become oligomerized and accumulate in brain tissue where they appear to cause neuronal injury and loss; indeed, some aver that such soluble intermediates of aggregation, or oligomers, are the key species that mediate toxicity and underlie seeding and spreading in disease (Cline et al. 2018, Choi and Gandhi 2018).

LBD is used here as an umbrella term that may include both Parkinson's disease dementia and dementia with Lewy bodies (DLB). Clinically these disorders manifest as parkinsonism together with cognitive and other neurobehavioral deficits. Dopaminergic treatments, especially at relatively large doses, improve motor features in some patients but generally to a lessor degree than in PD. DLB patients who display parkinsonian signs have severe DA deficiencies similar to those in those with PD. While both groups have striatal presynaptic dopamine transporter deficiencies, postsynaptic striatal DA receptors are reduced more in DLB than in PD, possibly contributing to the poorer response to LD treatment. Researchers do not know precisely why alpha-synuclein accumulates into Lewy bodies or how synuclein species can cause the symptoms of LBD. The formation of Lewy bodies has been considered to be a marker for PD; however, Lewy bodies have also have also been observed in approximately 60% of both sporadic and familial cases of Alzheimer's disease (AD) (Al-Mansoori et al. 2013). Accordingly, the aggregation of α-synuclein has been strongly implicated as a critical step in the development of neurodegenerative diseases (Al-Mansoori et al. 2013).

Sporadic PD or brainstem-predominant type LBD, and dementia with Lewy bodies (DLB) are the two most frequent α-synucleinopathies, and are progressive multisystem neurodegenerative disorders with widespread occurrence of α-synuclein deposits in the central, peripheral, and autonomic nervous system (Jellinger 2008). Reportedly, there is considerable clinical and pathologic overlap between PD (with or without dementia) and DLB, corresponding to Braak LB stages 5 and 6 (Braak et al. 2003), both frequently associated with variable Alzheimer-type pathology (Jellinger 2008).

Restless legs syndrome (RLS) is a PD-related disorder (Peeraully et al. 2012), that produces a strong urge to move one's legs. Characteristically, the unpleasant feeling in the legs can improve by moving them. These feelings generally occur while at rest and thus can interfere with sleep. Understandably, RLS patients may also experience daytime sleepiness, irritability and a depressed affect. RLS affects some 2.5 to 15% of the U.S. population. It becomes more common with age, and women are affected more often than men. The pathogenesis is unknown. Risk factors for RLS include PD, diabetes mellitus, rheumatoid arthritis, iron deficiency, kidney failure, and pregnancy. Several drug classes, among them antidepressants, antipsychotics and calcium channel blockers have also been linked to triggering this disorder. RLS may resolve if the underlying causal factors are addressed. Otherwise treatment includes certain lifestyle changes and medication such as levodopa or a dopamine agonist such as a DA-agonist. The pathophysiology of RLS is only partially understood. Multiple studies, however, implicate brain dopaminergic system dysfunction, especially projections from the A11 cell group to the spinal cord, possibly related to iron deficiency (Shiyi et al. 2017). In a post-mortem examination of RLS patients, Lewy bodies were not identified and α-synuclein immunohistochemistry was uniformly negative, thus suggesting that τ- or α-synuclein brain pathology is not a component of primary RLS (Pittok et al. 2004).

Dopamine-responsive dystonia syndrome (DDS) (Segawa Syndrome—Segawa et al 1976) is a genetic movement disorder that typically appears during early childhood. Symptoms include dystonic postures and movements along with parkinsonian features especially involving the lower extremities. These disabilities are often less severe in the morning and progressively worsen during the day (diurnal fluctuations) or upon exertion. The disorder responds well to levodopa treatment.

Progressive Supranuclear Palsy (“PSP”), the most common atypical parkinsonian syndrome, is characterized by akinesia, rigidity and postural instability as well as various non-parkinsonian symptoms. Standard dopaminergic treatments benefits some individuals with this disorder, bur usually not very much or for very long. The pathophysiology of PSP remains incompletely understood, although the dopamine neuron loss has been reported to occur mainly in the extranigral A10 midbrain cell groups rather than in the A9 group forming the nigral-striatal pathway as is the case in PD (Murphy et al. 2008).

Corticobasal Degeneration (“CBD”), pathologically classified as taupathy, presents with various phenotypes some of which include parkinsonian features, especially rigidity and akinesia (Reich and Grill 2009). Only in a few do these manifestations benefit from standard dose dopaminergic therapy, usually only to a moderate degree and short-lived duration, notwithstanding evidence of neuronal loss in the substantia nigra and a reduction in presynaptic dopamine transporter binding in the striatum.

Many now believe that protein misfolding processes leading to protein oligomerization and aggregation may be central to the cellular injury and destruction occurring in PD and PD-related disorders.

α-Synuclein and tau aggregates can co-exist in several neurodegenerative disorders, including Parkinson's disease, Alzheimer's disease, and progressive supranuclear palsy (PSP) and indeed there is evidence that α-synuclein enhances the harmful effects of tau, thus contributing to disease progression (Castillo-Carranza et al. 2018; Erro Aguirre et al. 2015). Tau oligomers in biological fluids, in particular in CSF, can be measured by ELISA and Western blot analysis using anti-tau oligomer antibodies (Sengupta et al. 2017).

The mechanism of alpha-synuclein aggregation in these disorders remains poorly understood. Current evidence suggests the conversion of a soluble alpha helical structure into a beta pleated conformation with subsequent oligomer formation leads to the aggregation, fibrillization and ultimately deposition of synuclein. Certain of the oligomeric forms of synuclein appear highly neurotoxic and could contribute to the neurodegenerative process characterizing PD and PD-related disorders. These characteristics are similar to the aberrant processing of prion proteins that also can become highly neurotoxic. In addition, phosphorylation of alpha-synuclein at the serine-129 residue has been implicated as a contributory factor (Chen et al. 2016). According to Prusiner et al. 2015, a prion form of alpha-synuclein could be a causal agent, especially for multiple system atrophy. Prions are small proteins that also can misfold, oligomerize, aggregate and propagate to other cells. The result in brain is a profound and spreading neurotoxic process.

Accordingly, inhibiting the initial misfolding, oligomerization and aggregation of certain brain proteins may be beneficial in slowing or even arresting the progression of PD and related disorders.

Current evidence further suggests that this aberrant misfolding, oligomerization, and fibrillization process also involves other brain proteins such as beta-amyloid and tau proteins and that certain of these abnormal species thus formed may play a role in the pathogenesis of disorders such as various tauopathies including PSP (Murphy et al. 2008), and FTLB (Nilson et al, 2017).

Accordingly, drugs that block the formation and/or neurotoxic action of these aberrant species may confer therapeutic benefit to patients suffering from these disorders, such as Alzheimer's disease and PD (Choi and Ghandi 2018; Nilson et al. 2017; Sengupta et al, 2017).

As mentioned above, alpha-synuclein, as well as other of the afore-mentioned oligomerized species, readily pass into extracellular spaces and have been identified in cerebrospinal fluid, blood, urine, and saliva (Marques and Outeiro 2012). The mechanisms of alpha-synuclein excretion are not fully understood, but studies have demonstrated that at least a fraction of alpha-synuclein is excreted within exosomes, the 40 nm to 100 nm vesicles of endocytic origin (reviewed in Shi et al. 2014). The ratio of monomeric to oligomeric species within exosomes in peripheral blood originating from the CNS could thus reflect disease transient intensity and/or cumulative severity (Shi et al. 2014), thus suggesting that peripheral blood exosomal alpha-synuclein and related species can help monitor the clinical state of a neurodegenerative disease and response to therapy. Similarly, alpha-synuclein levels in brain-derived exosomes have been reported to correlate with severity of impairment in cross-sectional samples from patients with LBD (Stuendl et al. 2016).

Based on the above, drugs that normalize the ratio of monomeric to oligomeric alpha-synuclein species in peripheral blood exosomes deriving from brain should slow or even arrest the neurodegenerative process associated with the synucleinopathies.

Various compositions for the treatment of PD and PD-related disorders that target the aggregation of brain proteins such as synuclein pathway have been proposed. The discovery process primarily involves cellular and animal models of prion- and synuclein-induced neurodegeneration (Prusiner et al. 2015; Visanji et al. 2016). Unfortunately, none of these models has been validated and all are currently regarded uncertain predictors of effects in humans. Nevertheless, these models continue to be widely used in the absence of better discovery techniques.

DA-agonists used to treat these PD-related disorders act by binding to and stimulating a particular set of DA receptor subtypes expressed in pathways coursing throughout the CNS. Neuronal DA receptors belong to a class of cell-surface proteins that responds to endogenous chemical signals (e.g., a neurotransmitter like DA) or to exogenous chemicals (e.g., drugs like DA-agonists) by changes in their electrical activity. Drugs of this type now used to treat PD-like disorders act on neuronal dopaminergic receptors, especially those of the D1, D2, and D3 subtypes. DA-agonists may be classified into various groups including partial and full agonists and indirect agonists. In most common use today for the treatment of neuropsychiatric disorders are the agonists considered to have essentially full efficacy at the DA receptor. A typical DA-agonist for this treatment is a DA-agonist. Full DA-agonists are now mainly employed in the treatment of PD and RLS. In contrast, partial DA-agonists, such as quinpirole, are rarely used in the treatment of PD and such indirect DA-agonists as amphetamine and/or dextroamphetamine, bupropion, and methylphenidate or dexmethylphenidate are mainly utilized therapeutically in such diverse conditions as ADHD, nicotine addiction, depression and narcolepsy.

Full DA-agonists, such as apomorphine (Apokin®), bromocriptine (Parlodel®), cabergoline (Dostinex®), ciladopa, dihydrexidine, lisuride, pergolide, pramipexole (Mirapex®), and rotigotine (Neupro®), on the other hand, have been used successfully for over 40 years to relieve symptoms of PD and PD-related disorders. In patients with early stage disease, drugs of this type often rival LD, the gold standard the treatment of PD, in efficacy. Moreover, considerable evidence suggests that their use in early stage patients may be safer than LD, since onset of Motor Complications (MC) occurs later when longer acting DA-agonists are used. In patients with later sage PD, who already suffering from MC, administration of longer acting DA-agonists tends to be associated with a diminished severity of MC compared with treatment with LD or a short duration agonist.

Unfortunately, DA-agonist usually must be administered along with LD in relatively advanced PD patients. This reflects the need to achieve the satisfactory degree of antiparkinsonian efficacy conferred by LD. Exacerbation of MC severity results.

What is needed is a drug with the efficacy of LD but the long action of most DA-agonists. Clearly, an oral preparation that combines the potentially long duration of action of many DA-agonists (thereby delaying onset of future MC or reducing the severity of existing MC) with the antiparkinsonian efficacy of levodopa would greatly advantage to those suffering from a PD-like disorder.

Motor complications, the most serious complication of the long-term treatment of PD with LD or a DA-agonist, manifest as fluctuations in the response to dopaminergic drugs and dyskinesias. The term fluctuations refers mainly to the progressive shortening of the motor response to each LD dose. Dyskinesias refer to the abnormal involuntary movements that may affect any part of the body. These complications appear in nearly half of PD patients within 5 years of treatment initiation and in 80-90% within 10 years. Characteristically, motor complications become nearly as disabling as symptoms of the underlying disease itself. The pathogenesis of motor fluctuations, especially those of the wearing-off type, has been linked to the progressive loss of DA stoppage capacity as a result of natural disease progression; dyskinesias appear to largely reflect changes in downstream striatal neurons as a result of their chronic intermittent, high intensity (non-physiologic) stimulation. Therapeutic options for MC remain limited and generally no more than marginally effectual.

Various factors have been proposed to explain the limited antiparkinsonian efficacy of DA-agonists as compared with LD. These include an action at the wrong set of DA receptors or conformational difference compared to the natural ligand that result in abnormal docking and thus the aberrant activation of down-stream signaling pathways. Nevertheless, despite strenuous effort spanning several decades, no DA-agonist or any other type drug, has ever been founds to be as effective as LD in relieving PD signs. One possibility, never adequately evaluated, is that agonist efficacy is limited by the inability to administer high enough dose levels. DA-agonists characteristically evidence a clear dose-response relation with respect to antiparkinsonian efficacy: higher doses confer greater efficacy. But, unfortunately, DA-agonists have a narrow therapeutic index, i.e., larger doses inevitably bring increased toxicity. Dose limiting adverse effects of these drugs typically involve the gastrointestinal system, especially manifesting as nausea and vomiting. A means to safely block these side effects might enable the administration of higher and thus more effective DA-agonist dosages in those who suffer from PD like disorders.

An example of a high emetogenic DA-agonist is apomorphine that has been proposed (US 2003/0073715, the content of which is incorporated herein in its entirety by reference) for ameliorating erectile dysfunction in psychogenic male patients. This document discloses that nausea side effects associated with the use of apomorphine can be substantially reduced by the pre-administration or co-administration of an antiemetic, as also reported in the Apokin® package insert recommending the use of trimethobenzamide as an antiemetic.

In US 2003/0073715, apomorphine is also proposed, alone or in association with an antiemetic such as prochlorperazine, in a layered tablet for sublingual administration or in a solution for spray nasal administration. This combination is acceptable for the sporadic use, but not for the treatment of PD and PD-related disorders needing a high-dose regimen of DA-agonist in a chronic treatment. In fact, this document does not make any distinction among the mechanisms of action of the antiemetics, for example by suggesting the use, as an antiemetic, of nicotine and, in particular, of prochlorperazine and domperidone that are DA-antagonists and thus potentially contraindicated in the treatment of PD and PD-related disorders such as MSA, DLB, LBD, RLS, DDS, FTLD, PSP, or CBD.

More than ten to fifteen years after these publications, no one though it useful to associate an AEsI, such as a peripheral DA-antagonist, a 5HT3-antagonist or a NK1-antagonist, with a DA-agonist, such as apomorphine or pramipexole, even at doses higher than the maximal recommended doses, for safely treating a PMND, in particular PD and PD-related disorders.

Thus, the problem of finding a safe as well as full effective treatment of these disorders is still unresolved.

### SUMMARY OF THE INVENTION

Considering the results of the above-cited, previously published studies in patients with PD indicating a dose-response relation between the amount of DA-agonist administered and the degree of motor improvement achieved in the range of doses studied clinically, it has been assumed that if dose limiting side effects of DA-agonists could be reduced or eliminated, then the administration of higher doses might provide a much needed increase the size of the therapeutic effect on parkinsonian symptoms but with a reduce risk of producing severe motor complications as a consequence of the characteristically longer duration of agonists' dopaminergic action, as compared to LD, long the standard of care in PD treatment, while at the same time having no significant deleterious effect on other aspects of safety or tolerability.

The therapeutic approach according to the present invention reverses the approach taught by the prior art, in the sense that it provides the use of an AEsI such as a peripheral DA-antagonist, a 5HT3-antagonist or a NK1-antagonist for an increase of the therapeutic effect of the DA-agonist (i.e., increase in antiparkinsonian efficacy) by concurrently globally counteracting their side effects as opposed to the prior art, teaching that DA-agonists (especially short acting ones like apomorphine) can be administered sporadically in combination with an anti-emetic to ameliorate an “off period” type of Motor Complications (MCs) but not intended to benefit parkinsonian symptoms and, in addition, acting to increase the risk of future MCs.

In addition, the literature recommends not using a 5HT3-antagonist for mitigating the nausea-vomiting caused by apomorphine (Chen J J 2011).

On the contrary, it has been found that the sporadic treatment with apomorphine may be safely conducted in combination with an AEsI such as a peripheral DA-antagonist, a 5HT3-antagonist or a NK1-antagonist and also that the chronic administration of said AEsI in combination with a DA-agonist, especially if it is appreciably long-acting, enables higher agonist doses and consequent greater antiparkinsonian efficacy.

It has also surprisingly been found that the peripheral DA-antagonists, in particular domperidone, known for its beneficial action on gastro-intestinal disorders, the 5HT3-antagonists and the NK1-antagonists, both known as antiemetics used for the prevention or treatment of post-operative nausea and vomiting and for the prevention or treatment of chemotherapy-induced nausea and vomiting, also act to reduce all the most frequent non-emetic AEs of DA-agonists, such as diaphoresis, lightheadedness and headache. Consequently, the peripheral DA-antagonists, the 5HT3-antagonists and the NK1-antagonists further enhance the tolerability of said DA-agonists, thus appearing as complete inhibitors of the AEs of the DA-agonists. Thus, said AEsI allows for a safe treatment of a PMND. For example, domperidone may be used in combination, including fixed-doses combinations, with pramipexole at doses higher, and even much higher, than its maximum recommended doses (daily and per unit form) for the treatment of a PMND.

In particular, it is actually possible to maximize the effects of prevention or treatment of a DA-agonist in improving the symptoms of PD and PD-related disorders in a patient suffering from said symptoms by administering to said patient said DA-agonist in combination with an AEsI, in particular at least one selected from the group consisting of a peripheral DA-antagonists, 5HT3-antagonists, and NK1-antagonists, that will act each as an antagonist at all relevant receptors not mediating an antiparkinsonian response, at doses that are not likely to introduce additional side effects.

In addition, by a combined AEsI/DA-agonist treatment, the maximization of the dopaminergic antiparkinsonian efficacy is achieved with DA-agonist doses higher, normally three-fold higher, and even much higher than the currently maximal tolerated ones and with AEsI doses equal to those currently used for preventing or treating nausea and vomiting of any nature, in particular in pediatric or adult patients undergoing cancer chemotherapy, without onset of clinically significant associated adverse effects.

It has been found that, by using an AEsI, in combination with pramipexole or a pharmaceutically acceptable salt or solvate thereof, it is possible to treat a patient suffering from a PD-related disorder such as RLS by maintaining a therapeutically effective pramipexole or pharmaceutically acceptable salt or solvate thereof daily dose with minimal adverse effect. More particularly, as set forth above, in the case of pramipexole dihydrochloride monohydrate, its combination with a peripheral DA-antagonist, a 5HT3-antagonist or a NK1-antagonist allows the administration of a therapeutic effective dose that, in many patients suffering from a PD-related disorder such as DDS, FTLD, PSP, and CGD, will significantly exceed the maximum recommended dose (4.5 mg/day) of pramipexole dihydrochloride monohydrate for the treatment of the motor symptoms of PD, thus increasing its efficacy in the treatment of said patients, including unexpectedly slowing disease progression.

For example, an AEsI, in particular a peripheral DA-antagonist such as domperidone, a 5HT3 antagonist such as ondansetron or dolasetron, or NK1-antagonist such as aprepitant or rolapitant, acts to effectively counteract the adverse effects associated with the administration of DA-agonists.

In summary, it has been found that, in combination with an AEsI, in particular of the peripheral DA-, 5HT3- and/or NK1-antagonist type, a DA-agonist acts by mimicking the levodopa/carbidopa action, with the main advantages of
being used at doses at least as high as, but also higher and, in some cases, even much higher than their maximum recommended doses indicated in the treatment of PD, thus providing a strong dopaminergic action;
generally having a longer duration of action in respect of levodopa/carbidopa; and thus
drastically reducing the adverse effects not only in respect of levodopa/carbidopa, but also in respect of their own adverse effects.

Consequently, said AEsIs are for use for safely treating PD and PD-related disorders in combination with a DA-agonist. For such a treatment, each AEsI is formulated in a pharmaceutical composition in dosage unit form to be administered to a patient in need of said treatment, in combination with said DA-agonist, also in a pharmaceutical composition in dosage unit form. Thus, the present invention also provides the use of an AEsI, especially of the peripheral DA-antagonist, 5HT3-antagonist and/or NK1-antagonist type, for the preparation of a medicament for the treatment of a PMND selected from the group consisting of PD and PD-related disorders such as MSA, DLB, LBD, RLS, DDS, FTLD, PSP, and CBD, in combination with a DA-agonist.

Clinical studies were conducted in subjects receiving a single oral dose of a DA-agonist, with or without a single oral dose of domperidone, ondansetron or aprepitant as representative AEsIs. The studies were carried out at a single center using a single-blind, cross-over design. Their objective was to demonstrate that currently approved doses of domperidone, ondansetron or aprepitant could safely attenuate the side effects of a DA-agonist given in doses equivalent or higher than those approved in the treatment of PD.

According to the present invention, pharmaceutical compositions comprising a pharmacologically active amount of at least one AEsI and a pharmacologically active amount of a DA-agonist, in admixture with pharmaceutical carriers, improve the symptoms of PD and PD-related disorders in patients suffering from said symptoms, even in the case of patients who have been withdrawn or are no longer benefitting from the DA-agonist therapy because of the severe side-effects, including those of the motor complication type. Thus assuring not only an improvement of the quality of life of the patients, but also an objective and previously unrealized improvement of their symptoms.

The present invention provides
the use of an AEsI for treating a protein misfolding neurodegenerative disease, in combination with a DA-agonist in a patient, or
a method for treating a protein misfolding neurodegenerative disease in a patient, which comprises administering to said patient in need of said treatment an effective daily dose of an AEsI, in combination with an effective daily dose of a DA-agonist.

In particular, the present invention provides the use of an AEsI for treating a disease selected from the group consisting of PD and PD-related disorders, in combination with a DA-agonist in a patient, or a method for treating a disease selected from the group consisting of PD and PD-related disorders in a patient, which comprises administering to said patient in need of said treatment an effective daily dose of an AEsI, in combination with a therapeutically effective daily dose of a DA-agonist, said effective daily dose of said DA-agonist being higher, normally up to three-fold higher, and even much higher than its maximum recommended daily dose.

Said DA-agonist is selected from the group consisting of apomorphine, bromocriptine, cabergoline, ciladopa, dihydrexidine, dihydroergocryptine, dinapsoline, doxanthrine, epicriptine, lisuride, pergolide, piribedil, pramipexole, propylnorapomorphine, quinagolide, romergoline, ropinirole, rotigotine, roxindole, sumanirole, and pharmaceutically acceptable salts and solvates and prodrugs of each of these DA-agonists.

According to a preferred embodiment, in said method (or use), said AEsI is preferably at least one selected from the group consisting of peripheral DA-antagonists, 5HT3-antagonists and NK1-antagonists, at an effective daily dose; said dopamine agonist is pramipexole or a pharmaceutically acceptable salt thereof, in an effective daily dose, for the treatment of a PD-related disorder selected from the group consisting of RLS, DDS, FTLD, PSP and CBD.

In particular, the addition of a DA-antagonist, a 5HT3-antagonist and/or a NK1-antagonist to pramipexole allows the administration of, for example, pramipexole doses (per unit form and daily) much higher than the maximum recommended pramipexole doses. Thus, for example, in a combination of a DA-antagonist, a 5HT3-antagonist and/or NK1-antagonist with pramipexole, the pramipexole daily doses, including pediatric daily doses and daily doses used in the titration period, are equivalent to from 0.375 mg to 45 mg of pramipexole dihydrochloride monohydrate.

According to an embodiment, in a combination of pramipexole with the peripheral DA-antagonist domperidone at a daily dose of from 2 mg to 120 mg, normally from 4 mg to 40 mg (including domperidone daily doses used in the pramipexole titration period), the pramipexole daily doses, including daily doses used in the titration period, are equivalent to from 0.375 mg to 45 mg of pramipexole dihydrochloride monohydrate.

According to this particular embodiment, in an adult patient, the pramipexole dihydrochloride monohydrate daily dose may be in a range equivalent to from more than 4.5 mg to 45 mg, from 6 mg to 45, and from more than 10 mg to 45 mg. Normally, said pramipexole dihydrochloride monohydrate daily dose in an adult patient is from 14.5 mg to 45 mg, from 15 mg to 35 mg, from 15 mg to 30 mg, from more than 20 mg to 25 mg, normally from 15 mg to 25 mg or from 20.25 mg to 25 mg.

These findings allow a safe treatment with pramipexole of a PMND such as PD or a PD-related disorder selected from the group consisting of RLS, DDS, FTLD, PSP, or CBD.

According to a preferred embodiment, this treatment is made by using a pharmaceutical composition comprising a pharmaceutical carrier or vehicle and a fixed-dose combination of domperidone or a pharmaceutically acceptable salt or solvate thereof, in an amount equivalent to from 2 mg to 120 mg of domperidone base and pramipexole or a pharmaceutically acceptable salt or solvate thereof, in an amount equivalent to from 0.125 mg to 45 mg. This pharmaceutical composition represents a further objet of the present invention.

Specifically, a peripheral DA-antagonist, a 5HT3-antagonist and/or a NK1-antagonist is for use in the treatment of a patient suffering from RLS in combination with pramipexole or a pharmaceutically acceptable salt or solvate thereof doses (per unit form and daily) equivalent to form from 0.125 mg to 6 mg or from 0.125 mg to 1 mg, normally from 0.125 mg to 0.75 mg or from 0.125 mg to 0.50 mg of pramipexole dihydrochloride monohydrate.

### DETAILED DESCRIPTION

The present invention relates to a pharmaceutical combination, including fixed-dose combinations, comprising a DA-agonists' AEsI Component (a), for use for the treatment of a PMND, in particular selected from the group consisting of PD and PD-related disorders such as MSA, DLB, LBD, RLS, DDS, FTLD, PSP, and CBD, in combination with a DA-agonist Component (b); and to the use of a pharmaceutical combination comprising said AEsI Component (a) and said DA-agonist Component (b), for the preparation of a medicament for treatment of a PMND selected from the group consisting of PD and PD-related disorders, wherein said DA-agonist in said combination is in an effective dose.

In particular, the invention provides
a DA-agonist-AEsI (AEsI) for use for the treatment of a disease selected from the group consisting of PD and PD-related disorders, in combination with a dopamine agonist;
the use of at least one AEsI (or an AEsI for use) for the treatment of a disease selected from the group consisting of PD and PD-related disorders, in combination with a dopamine agonist;
the use of at least one AEsI selected from the group consisting of peripheral dopamine antagonists (normally domperidone), 5HT3-antagonists and NK1-antagonists (or said AEsI for use) for the treatment of a disease selected from the group consisting of PD and PD-related disorders, in combination with a dopamine agonist;
the use at least one AEsI selected from the group consisting of peripheral dopamine antagonists (normally domperidone), 5HT3-antagonists and NK1-antagonists (or said AEsI for use) for the treatment of a disease selected from the group consisting of PD and PD-related disorders, in a fixed-dose combination with a dopamine agonist, wherein said dopamine adverse effects inhibitor is at least one AEsI selected from the group consisting of peripheral dopamine antagonists (normally domperidone), 5HT3-antagonists and NK1-antagonists, and the dopamine agonist is selected from the group consisting of pramipexole and pharmaceutically acceptable salt or solvate thereof, in admixture with a pharmaceutical carrier or vehicle, and said related disorder is Restless Leg Syndrome; and
a pharmaceutical composition for the treatment of restless leg syndrome comprising a pharmaceutical carrier or vehicle and a fixed-dose combination of at least one AEsI selected from the group consisting of peripheral DA-antagonists (normally domperidone), 5HT3-antagonists and NK1-antagonists, in an amount per unit form of from 1 μg to 600 mg and a DA-agonist selected from the group consisting of pramipexole and pharmaceutically acceptable salt or solvate thereof, in an amount per unit form equivalent to from 0.125 mg to 6 mg of pramipexole dihydrochloride monohydrate.

Thus, the present invention provides a pharmaceutical combination comprising a peripheral DA-antagonist, a 5HT-3 antagonist and/or a NK1-antagonist as Component (a); and a DA-agonist Component (b), for its use in the treatment of a disease selected from the group consisting of PD and PD-related disorders.

In this combination, the two components may be
each in a pharmaceutical composition in dosage unit form comprising, respectively, said Component (a) and said Component (b), each in admixture with a pharmaceutical carrier or vehicle, herein below also referred to as “combination (a/b)”; or
a mixture of Component (a) and Component (b), together, and a pharmaceutical carrier or vehicle, in a pharmaceutical composition in dosage unit form, in admixture with a pharmaceutical carrier or vehicle, herein below referred to as “Composition (ab)” or as “fixed-dose combination (ab)”

More particularly, the invention concerns, according to its aspects,
a method for the treatment of a disease selected from the group consisting of PD and PD-related disorders, in a patient in need of said treatment, which comprises administering to said patient at least one AEsI in combination with an effective DA-agonist daily dose;
at least one AEsI, for use for the treatment of a disease selected from the group consisting of PD and PD-related disorders in a patient in need of said treatment, in combination with an effective daily dose of a DA-agonist; and
the use of at least one AEsI for the manufacture of a medicament for the treatment of a disease selected from the group consisting of PD and PD-related disorders, in a patient in need of said treatment, in combination with an effective daily dose of DA-agonist.

The invention also provides the use at least one of said AEsI for the preparation of a medicament for the treatment of a disease selected from the group consisting of PD and PD-related disorders, in combination with a DA-agonist, said medicament being a fixed-dose combination, in particular a pharmaceutically composition in dosage unit form comprising, as an active ingredient, at least one of said AEsI and, as a second active ingredient, an effective dose per unit form of said DA-agonist, in admixture with a pharmaceutical carrier or vehicle, useful for the treatment of PD and PD-related disorders.

According to the present invention, the protective action of the peripheral DA-antagonist (normally domperidone), the 5HT3-antagonist, and/or the NK1-antagonist, said method (or use) assures at least a safe increase of the pramipexole daily doses with a consequent opportunity of arresting or at least slowing the progression of a PMND such as PD or a PD-related disorder in said patient.

The further benefit of an early discovery of the disease condition derives from ongoing studies directed at the determination of, for example, misfolded exosomal α-Synuclein in the human peripheral blood.

### The AEsI Component (a)

Any AEsI of the class of 5HT3-antagonists, of NK1-antagonists and/or of peripheral DA-antagonists (normally domperidone) can be used for the treatment of a PMND such as PD and PD-related disorders in combination with a DA-agonist, for allowing the full expression of efficacy of said DA-agonist in the treatment of PD and PD-related disorders. In some cases, in said combination, said AEsI allows for the administration of DA-agonist doses (daily or per unit form) that are higher, and even much higher than the maximum recommended or shown effective doses.

Said AEsI, in said combination, is used in a pharmaceutical composition in dosage unit form comprising said AEsI in an amount per unit form of from 1 μg to 600 mg, in admixture with a pharmaceutical carrier or vehicle and is administered at a daily dose of from 1 μg to 600 mg.

Preferably, in the aforementioned method, use and combination, including fixed-dose combinations, said AEsI is at least one AEsI selected from the group consisting of 5HT3-antagonists (A), NK1-antagonists, (B) and peripheral DA-antagonists (C), normally domperidone, at an effective daily dose; said dopamine agonist is pramipexole or a pharmaceutically acceptable salt thereof, at an effective daily dose; and said PMND is Parkinson's disease or a PD-related disorder is selected from the group consisting of Restless Leg Syndrome, dopamine-responsive dystonia syndrome, progressive supranuclear palsy, and corticobasal degeneration.

(A) The 5HT3-antagonist.

As set forth above, any of the 5HT3-antagonists disclosed in the literature may be used in combination with an effective daily dose of a DA-agonist. In particular, any of the 5HT3-antagonists disclosed in the literature may be used in combination with a daily dose a DA-agonist for the treatment of a disease selected from the group consisting of PD and PD-related disorders, as described in “The DA-agonist Component (b)” section below

The chronic use of this DA-agonist/AEsI combination, by mitigating or even eliminating adverse effects induced by the DA-agonist component, enables its high dose administration that slows progression of PD type disorders and more effectively reduces their symptoms.

The 5HT3-antagonist is preferably selected from the group consisting of 5-methyl-2-[(4-methyl-1H-imidazol-5-yl)methyl]-2,3,4,5-tetrahydro-1H-pyrido[4,3-b]indol-1-one (alosetron) and pharmaceutically acceptable salts and solvates thereof, especially its hydrochloride, disclosed in U.S. Pat. No. 5,360,800; N-(1-azabicyclo[2.2.2]octan-8-yl)-6-chloro-4-methyl-3-oxo-1,4-benzoxazine-8-carboxamide (azasetron) and pharmaceutically acceptable salts and solvates thereof, especially its hydrochloride, disclosed in U.S. Pat. No. 4,892,872; [(1S,5R)-8-methyl-8-azabicyclo[3.2.1]octan-3-yl] 3,5-dichlorobenzoate (bemesetron, CAS: 40796-97-2); (10R)-10-[(2-methyl-1H-imidazol-1-yl)methyl]-5,6,9,10-tetrahydro-4H-pyrido(3,2,1-jk)carbazol-11-one (cilansetron) and pharmaceutically acceptable salts and solvates thereof, especially its hydrochloride monohydrate, disclosed in U.S. Pat. No. 4,939,136; (3R)-10-oxo-8-azatricyclo[5.3.1.03,8]undec-5-yl 1H-indole-3-carboxylate (dolasetron) and pharmaceutically acceptable salts and solvates thereof, especially its monomethanesulfonate monohydrate, disclosed in U.S. Pat. No. 4,906,755; (+)-(R)-8,9-dihydro-10-methyl-7-[(5-methylimidazol-4-yl)methyl]pyrido[1,2-a]indol-6(7H)-one (fabesetron) and pharmaceutically acceptable salts and solvates thereof, especially its hydrochloride or maleate, disclosed in U.S. Pat. No. 5,141,945; 1-methyl-N-((1R,3r,5S)-9-methyl-9-azabicyclo[3.3.1]nonan-3-yl)-1H-indazole-3-carboxamide (granisetron) and pharmaceutically acceptable salts and solvates thereof, especially its hydrochloride, disclosed in U.S. Pat. No. 4,886,808; 2,3-dihydro-N-(8-methyl-8-azabicyclo[3.2.1]oct-3-yl)-2-oxo-1H-benzimidazole-1-carboxamide (itasetron) and pharmaceutically acceptable salts and solvates thereof, especially its hydrochloride, disclosed in U.S. Pat. No. 5,223,511; 1-phenylmethyl-2-(1-piperazinyl)-1H-benzimidazole (lerisetron) and pharmaceutically acceptable salts and solvates thereof, specially its hydrochloride, disclosed in U.S. Pat. No. 5,256,665 and, in a transdermal preparation, in U.S. Pat. No. 6,136,807; 6-fluoro-5-methyl-2-[(5-methyl-1H-imidazol-4-yl)methyl]-2,3,4,5-tetrahydro-1H-pyrido[4,3-b]indol-1-one (lurosetron, CAS 128486-54-4) and pharmaceutically acceptable salts and solvates thereof, especially its mesylate (GR 87442 N); (±) 1,2,3,9-tetrahydro-9-methyl-3-[(2-methyl-1H-imidazol-1-yl)methyl]-4H-carbazol-4-one (ondansetron) and pharmaceutically acceptable salts and solvates thereof, especially its hydrochloride dihydrate, disclosed in U.S. Pat. No. 4,695,578; (3aS)-2-[(S)-1-azabicyclo[2.2.2]oct-3-yl]-2,3,3a,4,5,6-hexahydro-1-oxo-1H-benz[de]isoquinoline (palonosetron) and pharmaceutically acceptable salts and solvates thereof, especially its hydrochloride, disclosed in U.S. Pat. No. 5,202,333; 1-methylindol-3-yl)-[(5R)-4,5,6,7-tetrahydro-3H-benzimidazol-5-yl]methanone (ramosetron) and pharmaceutically acceptable salts and solvates thereof, especially its fumarate, disclosed in U.S. Pat. No. 5,344,927; endo-N-(8-methyl-8-azabicyclo[3.2.1]oct-3-yl)-2,3-dihydro-3,3-dimethyl-indole-1-carboxamide (3,3-dimethyl-N-1αH,5αH-tropan-3α-yl-1-indolinecarboxamide, ricasetron, CAS 117086-68-7) and pharmaceutically acceptable salts and solvates thereof, especially its hydrochloride; the (3-endo)-8-methyl-8-azabicyclo[3.2.1]oct-3-yl ester of 1H-indole-3-carboxylic acid (3-tropanylindole-3-carboxylate, tropisetron) and pharmaceutically acceptable salts and solvates thereof, especially its hydrochloride, disclosed in U.S. Pat. No. 4,789,673; and 5-chloro-2,2-dimethyl-N-(8-methyl-8-azabicyclo[3.2.1]oct-3-yl)-2,3-dihydro-1-benzofuran-7-carboxamide (zatosetron) and pharmaceutically acceptable salts and solvates thereof, especially its maleate, disclosed in U.S. Pat. No. 5,563,148; the disclosures of all the US patents cited in this paragraph being incorporated herein in their entirety by reference.

Said 5HT3-antagonist is normally selected among those shown to be effective in or approved for the prevention or treatment of postoperative nausea and vomiting or for the prevention of the chemotherapy-induced nausea and vomiting.

Advantageously, said 5HT3-antagonist is selected from the group consisting of azasetron and pharmaceutically salts and solvates thereof, dolasetron and pharmaceutically acceptable salts and solvates thereof, granisetron and pharmaceutically salts and solvates thereof, ondansetron and pharmaceutically salts and solvates thereof, palonosetron and pharmaceutically salts and solvates thereof, ramosetron and pharmaceutically acceptable salts and solvates thereof, and tropisetron and pharmaceutically salts and solvates thereof.

Illustrative examples of pharmaceutically acceptable salts of these advantageous 5HT3-antagonists include acid addition salts with mineral or organic acids such as hydrochloric acid, hydrobromic acid, hydriodic acid, sulfuric acid, sulfamic acid, nitric acid, carbonic acid, phosphoric acid, formic acid, acetic acid, propionic acid, stearic acid, oxalic acid, malonic acid, succinic acid, fumaric acid, maleic acid, hydroxymaleic acid, lactic acid, malic acid, tartaric acid, citric acid, ascorbic acid, phenylacetic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid, 2,6-naphthalenedisulfonic acid, 1,5-naphthalenedisulfonic acid, aspartic acid, glutamic acid and pamoic (embonic) acid. Said salt may be solvated with a solvent, said solvent normally being water.

Antagonists of the 5HT3 receptor that are approved for the prevention or treatment of postoperative nausea and vomiting or for the prevention of the chemotherapy-induced nausea and vomiting are particularly useful according to the present invention. In particular, azasetron hydrochloride, commercially available in 10-mg tablets and in 10-mg vials for intravenous injection; dolasetron monomethanesulfonate monohydrate (also referred to as dolasetron mesylate), commercially available in 200-mg maximal dose tablets, and in 12.5 mg/0.625 ml vials; granisetron hydrochloride, commercially available in 2.24-mg maximal dose tablets; ondansetron hydrochloride dihydrate, commercially available in 10-mg maximal dose tablets and in a 2 mg/ml (in ondansetron base) solution available in 20-ml multidose vials; palonosetron hydrochloride, commercially available in 0.56-mg tablets and in 0.075 mg/1.5 ml or 0.25 mg/5 ml (in palonosetron base) vials; ramosetron hydrochloride, commercially available in 0.15 mg/ml for injection and in 0.1 mg oral tablets; and tropisetron hydrochloride, commercially available in 5.64-mg capsules, in 2.256 mg/2 ml vials for intravenous injection, and in 5.64-mg vials for intravenous or subcutaneous injection; are particularly advantageous 5HT3-antagonists.

According to the present invention, the 5HT3-antagonist is used in a pharmaceutical composition comprising, as an active ingredient, said 5HT3-antagonist in an amount per unit form of from 1 μg to 300 mg, in admixture with a pharmaceutical carrier or vehicle, and is administered at a daily dose of from 1 μg to 300 mg in combination with an effective dose of a DA-agonist.

Thus, for example, a pharmaceutical composition according to the present invention to be chronically administered in combination with a DA-agonist, may comprise a 5HT3-antagonist selected from the group consisting of azasetron and pharmaceutically acceptable salts and solvates and prodrugs thereof, in an amount per unit form equivalent to from 5 mg to 10 mg of azasetron hydrochloride, to be administered at a daily dose equivalent to from 15 mg to 40 mg of azasetron hydrochloride; dolasetron and pharmaceutically acceptable salts and solvates and prodrugs thereof, in an amount per unit form equivalent to from 25 mg to 200 mg of dolasetron mesylate, to be administered at a daily dose equivalent to from 75 mg to 200 mg of dolasetron mesylate; granisetron and pharmaceutically acceptable salts and solvates and prodrugs thereof, in an amount per unit form equivalent to from 0.5 mg to 2 mg granisetron base, to be administered at a daily dose equivalent to from 1.5 mg to 8 mg of granisetron base; ondansetron and pharmaceutically acceptable salts and solvates and prodrugs thereof, in an amount per unit form equivalent to from 0.5 mg to 32 mg, preferably from 0.5 mg to 16 mg, normally from 2 mg to 8 mg of ondansetron base, to be administered at a daily dose equivalent to from 6 mg to 64 mg, normally from 6 mg to 32 mg of ondansetron base; palonosetron and pharmaceutically acceptable salts and solvates and prodrugs thereof, in an amount per unit form equivalent to from 0.25 mg to 0.5 mg of palonosetron base, to be administered at a daily dose equivalent to from 0.75 to 2 mg of palonosetron base; ramosetron and pharmaceutically acceptable salts and solvates and prodrugs thereof, in an amount per unit form equivalent to from 0.05 mg to 0.2 mg of ramosetron hydrochloride, to be administered at a daily dose equivalent to from 0.05 mg to 0.2 mg of ramosetron hydrochloride; and tropisetron and pharmaceutically acceptable salts and solvates and prodrugs thereof, in an amount per unit form equivalent to from 2.5 mg to 5 mg of tropisetron base, to be administered at a daily dose equivalent to from 7.5 mg to 20 mg of tropisetron base.

Preferably, in said composition, said 5HT3-antagonist is selected from the group consisting of azasetron hydrochloride said 5HT3-antagonist is selected from the group consisting of azasetron hydrochloride, in an amount per unit form equivalent to from 5 mg to 10 mg to be administered at a daily dose equivalent to from 15 mg to 40 mg of azasetron hydrochloride; dolasetron mesylate, in an amount per unit form equivalent to from 25 mg to 200 mg of dolasetron mesylate, to be administered at a daily dose equivalent to from 75 mg to 200 mg; granisetron hydrochloride, in an amount per unit form equivalent to from 0.5 mg to 2 mg granisetron base, to be administered at a daily dose equivalent to from 1.5 mg to 16 mg, normally of from 2 mg to 8 mg; ondansetron hydrochloride dihydrate, in an amount equivalent to from 0.5 mg to 32 mg, normally from 2 mg to 32 mg, from 2 mg to 16 mg or from 2 mg to 8 mg ondansetron base, to be administered at a daily dose equivalent to from 6 mg to 64 mg, normally from 6 to 32 mg of ondansetron base; palonosetron hydrochloride, in an amount equivalent to from 0.25 mg to 0.5 mg palonosetron base, to be administered at a daily dose equivalent to from 0.75 to 2 mg of palonosetron base; ramosetron hydrochloride, in an amount per unit form of from 0.05 mg to 02 mg, to be administered at a daily dose of from 0.05 mg to 0.2 mg; and tropisetron hydrochloride, in an amount equivalent to from 2.5 mg to 5 mg tropisetron base, to be administered at a daily dose equivalent to from 7.5 mg to 20 mg of tropisetron base.

A composition comprising a 5HT3-antagonist as illustrated above is administered to a patient suffering from a disease selected from the group consisting of PD and PD-related disorders, in combination with an effective dose of a DA-agonist, also in a pharmaceutical composition in dosage unit form comprising an effective amount per unit of said DA-agonist, in admixture with a pharmaceutical carrier, as illustrated in “The-DA-agonist” section below, for improving the condition of said patient.

Said composition comprising a 5HT3-antagonist as described above is administered to a patient suffering from PD or a PD-related disorder such as MSA, DLB, LBD, RLS, DDS, FTLD, PSP, and CBD, in combination with a DA-agonist selected from the group consisting of apomorphine and pharmaceutically acceptable salts and solvates thereof, in an amount per unit dose equivalent to from 1 mg to 2 mg of apomorphine base, to be administered at a daily dose (in apomorphine base) of from 2 mg to 12 mg; bromocriptine and pharmaceutically acceptable salts and solvates thereof, in an amount per unit form equivalent to from 2.5 mg to 200 mg of bromocriptine base, to be administered at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, to be administered at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, to be administered at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, to be administered at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, to be administered at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; pramipexole, in an amount per unit form equivalent to from 0.125 mg to 45 mg of pramipexole dihydrochloride monohydrate, to be administered at a daily dose (in pramipexole dihydrochloride monohydrate) of from 0.125 mg to 45 mg, normally from 0.375 mg to 45 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 75 mg, normally from 0.125 mg to 25 mg, from 0.25 mg to 20 mg or from 0.25 mg to 15 mg of ropinirole base, to be administered at a daily dose (in ropinirole base) of from 0.25 mg to 75 mg, normally from 0.125 mg to 25 mg, from 0.25 mg to 20 mg or from 0.25 mg to 15 mg; and rotigotine and pharmaceutically acceptable salts and solvates thereof, to be administered at a daily dose equivalent to, from 2 mg to 24 mg of rotigotine base.

According to an embodiment, said composition comprising said 5HT3-antagonist as illustrated above, is administered to a patient suffering from a PD-related disorders selected from the group consisting of DDS, FTLD, PSP, and CBD, in combination with a DA-agonist selected from the group consisting of pramipexole and pharmaceutically acceptable salts and solvates thereof.

In particular, according to this embodiment, said 5HT3-antagonist in said composition is selected from group consisting of ondansetron and pharmaceutically acceptable salt and solvates and prodrugs thereof; and dolasetron and pharmaceutically acceptable salts and solvates and prodrugs thereof; and said dopamine agonist is selected from the group consisting of pramipexole and pharmaceutically acceptable salts and solvates thereof, in an amount per unit form equivalent to from 0.125 mg to 45 mg, normally from 1.5 mg to 25 mg or from 1.5 mg to 20 mg, of pramipexole dihydrochloride monohydrate.

More particularly, in said composition, said 5HT3-antagonist is selected from the group consisting of ondansetron and pharmaceutically acceptable salt and solvates and prodrugs thereof, in an amount per unit form equivalent to from 2 mg to 32 mg of ondansetron base, and dolasetron and pharmaceutically acceptable salts and solvates and prodrugs thereof, in an amount per unit form equivalent to 25 mg to 200 mg of dolasetron mesylate; and said dopamine agonist is pramipexole or a pharmaceutically acceptable salt thereof, in an amount per unit form equivalent to from more than 4.5 mg to 45 mg, normally from 4.5 mg to 25 mg or from 4.5 mg to 20 mg of pramipexole dihydrochloride monohydrate.

According to a specific embodiment of the present invention said composition comprising said 5HT3-antagonist as illustrated above, is administered to a patient suffering from SRL in combination with a DA-agonist selected from the group consisting of pramipexole and pharmaceutically acceptable salts and solvates thereof, also in a pharmaceutical composition comprising said pramipexole or a pharmaceutically acceptable salt or solvate in an amount equivalent to from 0.125 mg to 6 mg or from 0.125 mg to 1 mg, normally from 0.125 mg to 0.75 mg or from 0.125 mg to 0.50 mg of pramipexole dihydrochloride monohydrate, administered to said patient at a daily dose equivalent to from 0.125 mg to 6 mg or from 0.125 mg to 1 mg, normally from 0.125 mg to 0.75 mg or from 0.125 mg to 0.50 mg of pramipexole dihydrochloride monohydrate.

The pharmaceutical composition in dosage unit form comprising a 5HT3-antagonist as illustrated above may contain another active ingredient, in particular a DA-agonist, co-formulated with said 5HT3-antagonist, in admixture with a pharmaceutical carrier or vehicle in a fixed-dose combination Composition (ab).

The daily doses of the above 5HT3-antagonist Component (a) in said fixed dose combinations (ab), are illustrated above in this section.

The daily doses of the DA-agonists, in particular pramipexole Component (b) in said fixed dose combinations (ab) are illustrated below in “The DA-agonist Component (b)” section.

According to the present invention, a specific fixed-dose combination comprising or consisting of a pharmaceutical composition comprising a 5HT3-antagonist as disclosed above in this section and a DA-agonist selected from the group consisting of pramipexole and pharmaceutically acceptable salts thereof as disclosed in “The DA-agonist Component (b)” below, in admixture with a pharmaceutical carrier or vehicle, is administered to a patient suffering from a PD-related disorder selected from the group consisting of RLS, DDS, FTLD, PSP, and CBD for at least slowing the progression of said disorder.

(B) The NK1-antagonist

As set forth above, any NK1-antagonist known for its use as an AEsI is potentially useful for its combination with a DA-agonist for the treatment of a PD or a related disorder.

The chronic use of this DA-agonist/AEsI combination, by mitigating or even eliminating adverse effects induced by the DA-agonist component, enables its high dose administration that slows progression of PD and PD-related disorders and more effectively reduces their symptoms.

Advantageously, said NK1-antagonist is selected from the group consisting of
5-[[(2R,3S)-2-[(1R)-1-[3,5-bis(trifluoromethyl)phenyl]ethoxy]-3-(4-fluorophenyl)-4-morpholinyl]methyl]-1,2-dihydro-3H-1,2,4-triazol-3-one (aprepitant); described in U.S. Pat. No. 5,719,147, in a liquid oral formulation, in US 2017/0035774, and in an injectable emulsion in a single-dose vial for intravenous use containing 130 mg aprepitant in 18 ml of emulsion (Cinvanti®), described in U.S. Pat. No. 9,808,465 (the contents of each of which are incorporated herein in their entirety by reference);
[3-{[(2R,3S)-2-[(1R)-1-[3,5-bis(trifluoromethyl)phenyl]ethoxy]-3-(4-fluorophenyl)morpholin-4-yl]methyl}-5-oxo-2H-1,2,4-triazol-1-yl]phosphonic acid (fosaprepitant), disclosed, for example as meglumine salt in U.S. Pat. No. 5,691,336 and as di(cyclohexylamine) salt in US 2016/0355533, the contents of each of which are incorporated herein in their entirety by reference;
(2S,4S)-4-(4-Acetyl-1-piperazinyl)-N-[(1R)-1-[3,5-bis(trifluoromethyl)phenyl]ethyl]-2-(4-fluoro-2-methylphenyl)-N-methyl-1-piperidinecarboxamide (casopitant) described in U.S. Pat. No. 7,294,630, the contents of which are incorporated herein in its entirety by reference;
(2S)-1-[(3aS,4S,7aS)-4-hydroxy-4-(2-methoxyphenyl)-7,7-diphenyl-1,3,3a,5,6,7a-hexahydroisoindol-2-yl]-2-(2-methoxyphenyl)propan-1-one (INN: dapitant)
(2S,3S)—N-(5-tert-Butyl-2-methoxybenzyl)-2-(diphenylmethyl)-1-azabicyclo[2.2.2]octan-3-amine (maropitant), disclosed in U.S. Pat. No. 5,807,867, WO2005/082416 and EP 3173071 the contents of each of which are incorporated herein in their entirety by reference;
(2S,3S)-2-Diphenylmethyl-3-[(5-isopropyl-2-methoxybenzyl)amino]quinuclidine (INN: ezlopitant), disclosed by Evangelista S (2001). “Ezlopitant. Pfizer”; Current Opinion in Investigational Drugs: 2 (10): 1441-3; reviewed in Drugs: the Investigational Drugs Journal 6 (8): 758-72, the contents of each of which are incorporated herein in their entirety by reference;
(2S)—N-{2-[3,5-bis(trifluoromethyl)phenyl]ethyl}-2-[4-(cyclopropylmethyl)piperazin-1-yl]-N-methyl-2-phenylacetamide (INN: figopitant)
N-[(2R)-1-[Acetyl-[(2-methoxyphenyl)methyl]amino]-3-(1H-indol-3-yl)propan-2-yl]-2-(4-piperidin-1-ylpiperidin-1-yl)acetamide (INN: lanepitant);
2-[3,5-bis(trifluoromethyl)phenyl]-N,2-dimethyl-N-[4-(2-methylphenyl)-6-(4-methyl-1-piperazinyl)-3-pyridinyl]propanamide (netupitant), described in U.S. Pat. Nos. 6,297,375, 6,593,472, 6,719,996, and, in an oral composition, comprising 300 mg of netupitant and palonosetron hydrochloride in an amount equivalent to 0.5 mg of palonosetron base, herein below referred to as “netupitant-300/palonosetron-0.5”, in U.S. Pat. No. 8,951,969, the contents of each of which are incorporated herein in their entirety by reference;
{4-[5-{2-[3,5-bis(trifluoromethyl)phenyl]-N,2-dimethylpropanamido}-4-(2-methylphenyl)pyridin-2-yl]-1-methylpiperazin-1-ium-1-yl}methyl hydrogen phosphate (INN: fosnetupitant), described in WO 2013/082102 and, in a pure crystalline form, in US 2017/0096442, available in an injectable composition comprising fosnetupitant, in an amount of 235 mg, palonosetron hydrochloride, in an amount equivalent to 0.25 mg of palonosetron base (Akynzeo® for injection), herein below referred to as “fosnetupitant-235/palonosetron-0.25”, the contents of each of which are incorporated herein in their entirety by reference;
(2R,4S)-4-[(8aS)-6-oxo-1,3,4,7,8,8a-hexahydropyrrolo[1,2-a]pyrazin-2-yl]-N-[(1R)-1-[3,5-bis(trifluoromethyl)phenyl]ethyl]-2-(4-fluoro-2-methylphenyl)-N-methylpiperidine-1-carboxamide (INN: orvepitant), disclosed in US 2005/0176715 and, as crystalline maleate, in US 2011/0166150, the contents of each of which are incorporated herein in their entirety by reference;
(5S,8S)-8-({(1R)-1-[3,5-bis(trifluoromethyl)phenyl]ethoxy}methyl)-8-phenyl-1,7-diazaspiro[4.5]decan-2-one (rolapitant), described in U.S. Pat. No. 7,049,320 and, for an injectable form thereof, in U.S. Pat. No. 9,101,615, the contents of each of which are incorporated herein in their entirety by reference;
3-((3aR,4R,5S,7aS)-5-[(1R)-1-[3,5-bis(trifluoromethylphenyl]ethoxy]-4-(4-fluorophenyl)-1,3,3a,4,5,6,7,7a-octahydroisoindol-2-ylcyclopent-2-en-1-one (serlopitant) described in U.S. Pat. Nos. 7,544,815 and 7,217,731, the contents of each of which are incorporated herein in their entirety by reference;
(2S)—N-{(1R)-1-[3,5-bis(trifluoromethyl)phenyl]ethyl}-2-(4-fluoro-2-methylphenyl)-N-methylpiperazine-1-carboxamide (INN: vestipitant), described in WO 2001/25219 and, in intravenous formulation having a reduced tendency to cause hemolysis, in WO 2012/175434, the contents of each of which are incorporated herein in their entirety by reference; and
(2S,3S)—N-[(2-methoxy-5-[5-(trifluoromethyl)tetrazol-1-yl]phenylmethyl]-2-phenylpiperidin-3-amine (INN: vofopitant), disclosed by Gardner C J et al. Regul Pept. 1996 Aug. 27; 65(1):45-53, the contents of which are incorporated herein in their entirety by reference.

Illustrative examples of pharmaceutically acceptable salts of basic advantageous NK1-antagonists include acid addition salts with mineral or organic acids such as hydrochloric acid, hydrobromic acid, hydriodic acid, sulfuric acid, sulfamic acid, nitric acid, carbonic acid, phosphoric acid, formic acid, acetic acid, propionic acid, stearic acid, oxalic acid, malonic acid, succinic acid, fumaric acid, maleic acid, hydroxymaleic acid, lactic acid, malic acid, tartaric acid, citric acid, ascorbic acid, phenylacetic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid, 2,6-naphthalenedisulfonic acid, 1,5-naphthalenedisulfonic acid, aspartic acid, glutamic acid and pamoic (embonic) acid. Said salt may be solvated with a solvent, said solvent normally being water.

Illustrative examples of pharmaceutically acceptable salts of acidic NK1-antagonists such as fosaprepitant include salts with inorganic bases such as alkaline metal or alkaline-earth metal salts, and salts with organic bases such as dicyclohexylamine salts, N-methyl-D-glucamine (meglumine) salts, and salts with amino acids, as described in U.S. Pat. No. 5,691,336, the contents of which are incorporated herein in their entirety by reference.

An advantageous NK1-antagonist to be used in combination with a DA-agonist is selected from the group consisting of
aprepitant and pharmaceutically acceptable salts and solvates thereof,
fosaprepitant and pharmaceutically acceptable salts and solvates thereof,
casopitant and pharmaceutically acceptable salts and solvates thereof,
maropitant and pharmaceutically acceptable salts and solvates thereof,
eziopitant and pharmaceutically acceptable salts and solvates thereof,
lanepitant and pharmaceutically acceptable salts and solvates thereof,
netupitant and pharmaceutically acceptable salts and solvates thereof,
orvapitant and pharmaceutically acceptable salts and solvates thereof,
rolapitant and pharmaceutically acceptable salts and solvates thereof,
serlopitant and pharmaceutically acceptable salts and solvates thereof,
vestipitant and pharmaceutically acceptable salts and solvates thereof,
vofopitant and pharmaceutically acceptable salts and solvates thereof,
netupitant-300/palonosetron-0.5, and
fosnetupitant-235/palonosetron-0.25.

Said NK1-antagonist is normally selected among those shown to be effective in or approved for the prevention or treatment of postoperative nausea and vomiting or for the prevention of the chemotherapy-induced nausea and vomiting.

Aprepitant, fosaprepitant meglumine, fosaprepitant di(cyclohexylamine), rolapitant, rolapitant hydrochloride, netupitant-300/palonosetron-0.5, and fosnetupitant-235/palonosetron-0.25 are particularly advantageous NK1-antagonists.

Fosaprepitant, fosaprepitant meglumine, and fosaprepitant di(cyclohexylamine), are prodrugs of aprepitant, and fosnetupitant is a prodrug of netupitant. Thus, the expressions “aprepitant and pharmaceutically acceptable salts and solvates and prodrugs thereof” and “netupitant and pharmaceutically acceptable salts and solvates and prodrugs thereof” include
aprepitant, fosaprepitant, fosaprepitant meglumine, fosaprepitant di(cyclohexylamine), and other salts or solvates of aprepitant or fosaprepitant; and, respectively,
netupitant, fosnetupitant, and salts or solvates of aprepitant and fosaprepitant.

Antagonists of the NK1 receptor that are approved for the prevention or treatment of postoperative nausea and vomiting or for the prevention of chemotherapy-induced nausea and vomiting are particularly useful according to the present invention. In particular, aprepitant is commercially available (Emend®) in capsules containing 40 mg, 80 mg, or 125 mg aprepitant or, as fosaprepitant dimeglumine (Emend® Injection), in vials containing 115 mg or 150 mg fosaprepitant; rolapitant is available (Varubi®) in 90-mg tablets; netupitant is available (Akynzeo®) in a fixed-dose combination in capsules containing 300 mg of netupitant and 0.5 mg of the 5HT3-antagonist palonosetron (as hydrochloride), herein below referred to as “netupitant-300 mg/palonosetron-0.5 mg”, and fosnetupitant is available (Akynzeo® for injection) in a fixed-dose combination in vials containing 235 mg of netupitant and 0.25 mg of the 5HT3-antagonist palonosetron (as hydrochloride), herein below referred to as “netupitant-235/palonosetron-0.25”, in single-dose vial for reconstitution for intravenous injection. Each of these preparations is a particularly advantageous NK1-antagonist for the combination with the DA-agonist, in particular with pramipexole.

In the aforementioned method, use and combination, including fixed-dose combinations, said NK1-antagonist is present in an amount per unit form and is administered at a daily dose of from 1 μg to 600 mg, normally from 1 mg to 600 mg, or from 1 mg to 300 mg.

In particular, said NK1-antagonist is selected from group consisting of aprepitant and pharmaceutically acceptable salt and solvates and prodrugs thereof, such as fosaprepitant; and rolapitant and pharmaceutically acceptable salts and solvates and prodrugs thereof; and said dopamine agonist is pramipexole or a pharmaceutically acceptable salt thereof at a daily dose equivalent to from 0.375 mg to 45 mg, normally from 1.5 mg to 45 mg, of pramipexole dihydrochloride monohydrate.

More particularly, in said combination, said NK1-antagonist is selected from the group consisting of aprepitant and pharmaceutically acceptable salts and solvates thereof, in an amount per unit form and at a daily dose equivalent to from 10 mg to 250 mg of aprepitant; fosaprepitant and pharmaceutically acceptable salts and solvates thereof, in an amount per unit form and at a daily dose equivalent to from 10 mg to 250 mg of aprepitant; rolapitant and pharmaceutically acceptable salts and solvates thereof, in an amount per unit form and at a daily dose equivalent to from 15 mg to 270 mg of rolapitant, netupitant and pharmaceutically acceptable salts and solvates thereof, in an amount per unit form and at a daily dose equivalent to from 300 mg to 600 mg; netupitant-300/palonosetron-0.5 and fosnetupitant-235/palonosetron-0.25.

Preferably, in said combination, said NK1-antagonist is selected from the group consisting of aprepitant and pharmaceutically acceptable salt and solvates and prodrugs thereof at a daily dose equivalent to from 10 mg to 250 mg of aprepitant base, and rolapitant and pharmaceutically acceptable salts and solvates and prodrugs thereof, at a daily dose equivalent to from 15 mg to 270 mg of rolapitant base; and said dopamine agonist is pramipexole or a pharmaceutically acceptable salt thereof at a daily dose equivalent to from 0.375 mg to 45 mg, normally from 1.5 mg to 45 mg, of pramipexole dihydrochloride monohydrate.

For its administration to a patient suffering from a PD or a related disorder, in combination with a DA-agonist, in particular pramipexole, each of the above NK1-antagonists is formulated in a pharmaceutical composition in dosage unit form comprising, as an active ingredient, said NK1-antagonist, in admixture with a pharmaceutical carrier or vehicle.

As set forth above, said pharmaceutical compositions comprises said NK1-antagonist in an amount per unit form of from 1 μg to 600 mg.

Such a composition comprising a NK1-antagonist is to be administered to a patient suffering from PD or a related disorder, in combination with an effective dose of a DA-agonist, also in a pharmaceutical composition in dosage unit form comprising an effective amount per unit form of said DA-agonist, in admixture with a pharmaceutical carrier, as illustrated in “The-DA-agonist” section below, for improving the condition of a patient with Parkinson's disease and related Parkinsonian syndromes.

Said composition comprising said NK1-antagonist as illustrated above is administered once a day to a patient suffering from PD or a PD-related disorder such as MSA, DLB, LBD, RLS, DDS, FTLD, PSP, and CBD, in combination with a DA-agonist selected from the group consisting of apomorphine and pharmaceutically acceptable salts and solvates thereof, in an amount per unit dose equivalent to from 1 mg to 2 mg of apomorphine base, to be administered at a daily dose (in apomorphine base) of from 2 mg to 12 mg; bromocriptine and pharmaceutically acceptable salts and solvates thereof, in an amount per unit form equivalent to from 2.5 mg to 200 mg of bromocriptine base, to be administered at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, to be administered at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, to be administered at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, to be administered at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, to be administered at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; pramipexole, in an amount per unit form equivalent to from 0.125 mg to 45 mg of pramipexole dihydrochloride monohydrate, to be administered at a daily dose (in pramipexole dihydrochloride monohydrate) of from 0.375 mg to 45 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 75 mg, normally from 0.125 mg to 25 mg, from 0.25 mg to 20 mg or from 0.25 mg to 15 mg of ropinirole base, to be administered at a daily dose (in ropinirole base) of from 0.25 mg to 75 mg, normally from 0.125 mg to 25 mg, from 0.25 mg to 20 mg or from 0.25 mg to 15 mg; and rotigotine and pharmaceutically acceptable salts and solvates thereof, to be administered at a daily dose equivalent to, from 2 mg to 24 mg of rotigotine base.

According to an embodiment, said composition comprising said NK1-antagonist as illustrated above, is administered to a patient suffering from a PD-related disorders selected from the group consisting of DDS, FTLD, PSP, and CBD, in combination with a DA-agonist selected from the group consisting of pramipexole and pharmaceutically acceptable salts and solvates thereof.

Aprepitant and pharmaceutically acceptable salts and solvates and prodrugs thereof, netupitant and pharmaceutically acceptable salts and solvates and prodrugs thereof and rolapitant and pharmaceutically acceptable salts and solvates and prodrugs thereof are particularly preferred NK1-antagonists of the invention.

In particular, according to this embodiment, said NK1-antagonist in said composition is selected from group consisting of aprepitant and pharmaceutically acceptable salt and solvates and prodrugs thereof, such as fosaprepitant; netupitant and pharmaceutically acceptable salts and solvates and prodrugs thereof, such as fosnetupitant, and rolapitant and pharmaceutically acceptable salts and solvates and prodrugs thereof; and said DA-agonist is pramipexole or a pharmaceutically acceptable salt thereof in an amount per unit form of from 0.125 mg to 45 mg, normally from 0.125 mg to 25 mg or from 1.5 to mg to 20 mg. of pramipexole dihydrochloride monohydrate.

More particularly, in said composition, said NK1-antagonist active ingredient of said pharmaceutical composition is selected from the group consisting of aprepitant and pharmaceutically acceptable salts and solvates thereof, in an amount, per unit form, equivalent to from 10 mg to 250 mg of aprepitant; fosaprepitant and pharmaceutically acceptable salts and solvates thereof, in an amount, per unit form, equivalent to from 10 mg to 250 mg of aprepitant; rolapitant and pharmaceutically acceptable salts and solvates thereof, in an amount, per unit form, equivalent to from 15 mg to 270 mg of rolapitant; netupitant and pharmaceutically acceptable salts and solvates thereof, in an amount, per unit form, equivalent to from 300 mg to 600 mg; netupitant-300/palonosetron-0.5, and fosnetupitant-235/palonosetron-0.25; and said dopamine agonist is pramipexole or a pharmaceutically acceptable salt thereof, in an amount per unit form equivalent to from more than 4.5 mg to 45 mg, normally from more than 4.5 mg to 25 mg or from more than 4.5 mg to 20 mg of pramipexole dihydrochloride monohydrate.

According to a specific embodiment of the present invention, said composition comprising said NK1-antagonist is administered to a patient suffering from SRL in combination with a DA-agonist selected from the group consisting of pramipexole and pharmaceutically acceptable salts and solvates thereof, also in a pharmaceutical composition comprising said pramipexole or pharmaceutically acceptable salts and solvates thereof in an amount per unit form equivalent to form from 0.125 mg to 6 mg or from 0.125 mg to 1 mg, normally from 0.125 mg to 0.75 mg or from 0.125 mg to 0.50 mg of pramipexole dihydrochloride monohydrate.

In order to assure a sure, safe and concurrent administration of said NK1-antagonist and said DA-agonist, the present invention provides a fixed-dose combination consisting of a pharmaceutical composition in dosage unit form Composition (ab) comprising an effective amount per unit form of said NK1-antagonist and an effective amount per unit form of said DA-agonist, in admixture with a pharmaceutical carrier or vehicle for the treatment of PD and PD-related disorders.

The daily doses of the above Component (a) in said fixed dose combination (ab) are illustrated above in this section.

The daily doses of the above Component (b) in said fixed dose combination (ab) are illustrated below in the “DA-agonist Component (b)” section.

According to the present invention, a specific fixed-dose combination comprising or consisting of a pharmaceutical composition comprising a NK1-antagonist as disclosed above in this section and a DA-agonist selected from the group consisting of pramipexole and pharmaceutically acceptable salts thereof as disclosed in “The DA-agonist Component (b) below, in admixture with a pharmaceutical carrier or vehicle is administered to a patient suffering from a PD-related disorder selected from the group consisting of RLS, DDS, FTLD, PSP, and CBD for at least slowing the progression of said disorder.

### (C) The Peripheral DA-Antagonist Domperidone

Domperidone, 5-chloro-1-(1-[3-(2-oxo-2,3-dihydro-1H-benzo[d]imidazol-1-yl)propyl]piperidin-4-yl)-1H-benzo[d]imidazol-2(3H)-one

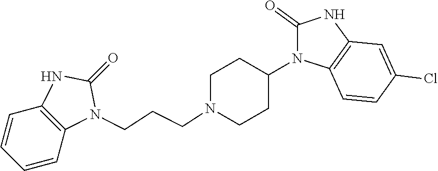 [SMILES: O=c1[nH]c2ccccc2n1CCCN1CCC(n2c(=O)[nH]c3cc(Cl)ccc32)CC1]

is a dopamine antagonist that is reputed not to enter the CNS (Brogden et al. 1982).

It is used for the treatment of gastrointestinal disorders, in particular for improving the delayed gastric emptying of functional origin with gastro-esophageal reflux and/or dyspepsia, for controlling nausea and vomiting of central or local origin, as an anti-emetic in patients receiving cytostatic and radiation therapy; and for facilitating radiological examination of the upper gastro-intestinal tract.

A study to investigate the effects of the D₂-receptor agonist pramipexole (at a dose of 0.5 mg/day) with and without the co-administration of the peripherally acting D₂-receptor antagonist domperidone (at a daily dose of 40 mg), on measures of alertness, autonomic and endocrine function, reports that a high enough concentration of the drug crosses the blood-brain barrier to partially antagonize some of the autonomic actions of pramipexole (Samuels et al. 2007). In particular, this report provides a cautionary note to the use of domperidone alongside pramipexole where the results of interest are those from pramipexole alone.

Domperidone is not approved in the USA, but the Food and Drug Administration (FDA) recognized that there are some patients with severe gastrointestinal motility disorders that are difficult to manage with available therapy, who may benefit from domperidone and in whom its potential benefits outweigh its risks (FDA Domperidone IND Packet. For Sponsors Treating Patients with Gastrointestinal Disorders).

By combining domperidone with a DA-agonist, for example pramipexole according to the present invention, not only does the pramipexole effect size become clinically significant by using domperidone and pramipexole doses falling into the range considered safe and tolerable for human subjects by concurrently and safely interdicting the basic degenerative process of the PMND such as AD, PD or a PD-related disorder in patients, but it is also possible to increase the pramipexole dose to a high degree.

The domperidone Component (a) is selected from the group consisting of domperidone base and pharmaceutically acceptable salt and solvates thereof.

Illustrative examples of pharmaceutically acceptable salts of domperidone include acid addition salts with, for example, hydrochloric acid, hydrobromic acid, sulfuric acid, nitric acid, phosphoric acid, acetic acid, propanoic acid, hydroxyacetic acid, 2-hydroxypropanoic acid, 2-oxopropanoic acid, propanedioic acid, butanedioic acid, (Z)-2-butenedioic acid (fumaric acid), (E)-2-butenedioic acid (maleic acid), 2-hydroxybutanedioic acid, 2,3-dihydroxybutanedioic acid, 2-hydroxy-1,2,3-propanetricarboxylic acid, benzoic acid, 3-phenyl-2-propenoic acid, .alpha.-hydroxybenzeneacetic acid, methanesulfonic acid, ethanesulfonic acid, benzenesulfonic acid, 4-methylbenzensulfonic acid, cyclohexanesulfamic acid, 2-hydroxybenzoic acid, 4-amino-2-hydroxybenzoic acid. These salts are disclosed in U.S. Pat. No. 4,066,772, the contents of which are incorporated herewith in their entirety for reference. The solvation solvent is normally water.

A butanedioic acid addition salt, domperidone succinate 1:1, is particularly interesting because it could improve domperidone bioavailability, particularly in the fed state, and may reduce inter-patient variability, thus being advantageous for a pharmaceutical use (Bruni et al. 2013). Also domperidone maleate is an advantageous domperidone salt.

For its administration to a patient suffering from a PD or a PD-related disorder, in combination with a DA-agonist, in particular pramipexole, domperidone or a pharmaceutically acceptable salt or solvate thereof Component (a) is formulated in a pharmaceutical composition in dosage unit form comprising, as an active ingredient, said domperidone or pharmaceutically acceptable salt or solvate thereof, in admixture with a pharmaceutical carrier or vehicle.

An advantageous domperidone Component (a) is selected from the group consisting of domperidone base, domperidone maleate and domperidone succinate (1:1).

Said pharmaceutical compositions comprises said domperidone or a pharmaceutically acceptable salt or solvate thereof in an amount per unit form equivalent to from 2 mg to 120 mg, normally from 2 mg to 40 mg of domperidone base.

Such a composition comprising domperidone or a pharmaceutically acceptable salt or solvate thereof is to be administered to a patient suffering from PD or a PD-related disorder, in combination with an effective daily dose (normally from 0.025 mg to 1000 mg) of a DA-agonist, also in a pharmaceutical composition in dosage unit form comprising an effective amount per unit form (normally from 0.001 mg to 200 mg) of said DA-agonist, in admixture with a pharmaceutical carrier, as illustrated in “The-DA-agonist” section below, for improving the condition of a patient with Parkinson's disease or a related Parkinsonian syndrome.

Preferably, said composition comprising domperidone is to be administered once to three times per day, at a daily dose equivalent to from 4 mg to 120 mg, normally from 4 mg to 40 mg of domperidone base, to a patient suffering from PD or a PD-related disorder, in combination with a DA-agonist.

In particular, according to the present invention, domperidone may be used in the above pharmaceutical compositions in an amount per unit form within the above range, in particular in amount per unit form equivalent to from 2 mg to 120 mg, from 2 mg to 100 mg, from 2 mg to 80 mg, from 2 mg to 60 mg, from 2 mg to 40 mg, from 2 mg to 30 mg, or from 2 mg to 20 mg, normally from 10 mg to 100 mg, from 10 mg to 80 mg, from 10 mg to 60 mg or from 10 mg to 40 mg, from 10 mg to 30 mg, or from 10 mg to 20 mg of domperidone base.

The amount of domperidone in an IR-formulated Unit Form is normally equivalent to from 2 mg to 60 mg of domperidone base, in particular equivalent to from 2 mg to 50 mg, from 2 mg to 40 mg, from 2 mg to 30 mg, from 2 mg to 25 mg, from 2 mg to 20 mg, from 2 mg to 15 mg or from 2 mg to 10 mg of domperidone base, advantageously from 10 mg to 60 mg, from 10 mg to 40 mg, from 10 mg to 30 mg, from 10 mg to 25 mg, from 10 mg to 20 mg, or from 10 mg to 15 mg of domperidone base.

The amount of domperidone in an ER-formulated Unit Form is normally equivalent to from 4 mg to 120 mg, in particular equivalent to from 4 mg to 100 mg, from 4 mg to 80 mg, from 4 mg to 60 mg, from 4 mg to 50 mg, from 4 mg to 40 mg, from 4 mg to 30 mg or from 4 to 20 mg of domperidone base, normally equivalent to from 10 mg to 120 mg, from 10 to 100 mg, from 10 to 80 mg, from 10 mg to 60 mg from 10 to 50 mg, from 10 mg to 40 mg, from 10 to 30 mg or from 10 mg to 20 mg of domperidone base.

Using this compositions, domperidone is administered to a patient at a daily dose equivalent to from 4 mg to 120 mg, in particular equivalent to from 4 mg to 100 mg, from 4 mg to 80 mg, from 4 mg to 60 mg or from 4 mg to 40 mg, from 4 mg to 30 mg, or from 4 mg to 20 mg, normally from 10 mg to 100 mg, from 10 mg to 80 mg, from 10 mg to 60 mg, from 10 mg to 40 mg, from 10 mg to 30 mg, or from 10 mg to 20 mg of domperidone base.

Said DA-agonist is normally selected from the group consisting of apomorphine and pharmaceutically acceptable salts and solvates thereof, in an amount per unit dose equivalent to from 1 mg to 2 mg of apomorphine base, to be administered at a daily dose (in apomorphine base) of from 2 mg to 12 mg; bromocriptine and pharmaceutically acceptable salts and solvates thereof, in an amount per unit form equivalent to from 2.5 mg to 200 mg of bromocriptine base, to be administered at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, to be administered at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, to be administered at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, to be administered at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, to be administered at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; pramipexole, in an amount per unit form equivalent to from 0.125 mg to 45 mg of pramipexole dihydrochloride monohydrate, to be administered at a daily dose (in pramipexole dihydrochloride monohydrate) of from 0.375 mg to 45 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 20 mg of ropinirole hydrochloride, to be administered at a daily dose (in ropinirole hydrochloride) of from 0.25 mg to 20 mg; and rotigotine and pharmaceutically acceptable salts and solvates thereof, to be administered at a daily dose equivalent to, from 2 mg to 24 mg of rotigotine base.

According to a preferred embodiment, for the administration to a patient suffering from PD or a PD-related disorder,
said DA-antagonist is domperidone or a pharmaceutically acceptable salt thereof, at daily dose equivalent to from 4 mg to 120 mg, normally from 4 mg to 40 mg of domperidone base;
said DA-agonist is pramipexole or a pharmaceutically acceptable salt thereof, at a daily dose equivalent to from 0.125 mg to 45 mg or from 0.375 mg to 45 mg, normally from 1.5 mg to 25 mg or from 1.5 mg to 20 mg, of pramipexole dihydrochloride monohydrate; and
said PMND is PD or a PD-related disorder is selected from the group consisting of MSA, DLB, LBD, RLS, DDS, FTLD, PSP, and CBD.

According to this preferred embodiment, in particular for the treatment of a PD-related disorder such as MSA, DLB, LBD, DDS, FTLD, PSP, and CBD, the daily dose of said pramipexole or a pharmaceutically acceptable salt thereof is equivalent to from more than. 4.5 mg to 45 mg, normally from more than 4.5 mg to 25 mg or from more than 4.5 mg to 20 mg, of pramipexole dihydrochloride monohydrate. As set forth above, by combining domperidone with pramipexole according to the present invention, not only does the pramipexole effect size become clinically significant by using pramipexole doses falling into the range considered safe and tolerable for human subjects by concurrently and safely interdicting the basic degenerative disease process in patients, but it is also possible to increase the pramipexole dose to a high degree.

In the case of the treatment of RLS, as a specific PD-related disorder, daily dose of said pramipexole or a pharmaceutically acceptable salt thereof is equivalent to from 0.125 mg to 6 mg.

In said combination with The DA-agonist Component (b), domperidone Component (a) may also be formulated in a fixed-dose combination (ab), comprising an effective amount of domperidone, and an effective amount of DA-agonist, in admixture with a pharmaceutical carrier or vehicle.

A fixed-dose combination (ab) comprises or consists of a pharmaceutical composition in dosage unit form comprising, as an active ingredient, said domperidone Component (a), in the above-illustrated amount per unit form; and, as a second active ingredient, pramipexole Component (b), in an effective amount per unit form as illustrated in “The DA-agonist Component (b)” section below, in admixture with a pharmaceutical carrier or vehicle. Said fixed-dose combination is destined to be administered to a patient suffering from PD or a related disorder.

The daily doses of the above Component (a) in said fixed dose combination (ab) are illustrated above in this section.

The daily doses of the above Component (b) in said fixed dose combination (ab) are illustrated below in the “DA-agonist Component (b)” section below.

A preferred fixed-dose combination (ab) comprises or consists of a pharmaceutical composition comprising, as an active ingredient, domperidone Component (a), in an amount equivalent to from 2 mg to 120 mg of domperidone base; and, as a second active ingredient, pramipexole Component (b), in an amount equivalent to from 0.125 mg to 45 mg of pramipexole dihydrochloride monohydrate, in admixture with a pharmaceutical carrier or vehicle.

A specific fixed-dose combination (ab) comprises or consists of a pharmaceutical composition comprising
(a) domperidone or a pharmaceutically acceptable salt or solvate thereof, in an amount equivalent to from 2 mg to 120 mg of domperidone base; and,
(b) pramipexole or a pharmaceutically acceptable salt or solvate thereof, in an amount equivalent to from more than 4.5 mg to 45 mg, normally from 15 mg to 25 mg or from more than 20 mg to 25 mg of pramipexole dihydrochloride monohydrate,
   in admixture with a pharmaceutical carrier or vehicle.

Said composition normally is in dosage unit form, and the above domperidone and pramipexole amounts are per unit form.

A composition comprising Component (a), in combination with a composition comprising Component (b), or in the fixed-dose combination Composition (ab) is safely administered to a patient for treating protein misfolding neurodegenerative diseases which benefit from drugs that augment dopamine mediated neurotransmission in the nervous system, in particular PD and PD-related disorders.

According to an embodiment, the invention provides a pharmaceutical composition for use for the treatment of a protein misfolding neurodegenerative disease (PMND) in a patient, which comprises a pharmaceutical carrier or vehicle and a fixed-dose combination of a dopamine-agonist Adverse Effects (or Events) Inhibitor (AEsI) selected from the group consisting of domperidone and pharmaceutically acceptable salts and solvates thereof, in an amount equivalent to from 2 mg to 120 mg of domperidone base, and a dopamine agonist selected from the group consisting of pramipexole and pharmaceutically acceptable salts and solvates thereof, in an amount equivalent to from 0.125 mg to 45 mg of pramipexole dihydrochloride monohydrate.

According to a specific embodiment, for the use of the AEsI (or in the method using the AEI) for treating RLS in a patient according to the present invention, the AEsI/DA-agonist combination comprises or consists of an AEsI selected from the group consisting of domperidone base, domperidone maleate and domperidone succinate (1:1), at an effective daily dose equivalent to from 4 mg to 40 mg of domperidone base, in combination with a DA-agonist selected from the group consisting of pramipexole base and pramipexole dihydrochloride monohydrate, at an effective daily dose equivalent to from 0.125 mg to 6 mg, from 0.125 mg to 1 mg, from 0.125 mg to 0.75 mg, or from 0.125 mg to 0.5 mg of pramipexole dihydrochloride monohydrate.

Herein below, the above 5HT3-antagonists (A), NK1-antagonists (B), and the peripheral DA-antagonist domperidone, each with its doses per unit form and daily doses, will also globally referred to as “(A)-(C)AEsIs”.

### The DA-agonist Component (b)

Any substance having shown to activate dopamine receptors, imitating the physiological response of the natural neurotransmitter, and studied or commercially available for their effects in treating Parkinson's disease or hyperprolactinemia may be used according to the present invention.

An advantageous DA-agonist is selected from the group consisting of
(6aR)-6-methyl-5,6,6a,7-tetrahydro-4H-dibenzo[de,g]quinoline-10,11-diol (apomorphine) and pharmaceutically acceptable salts and solvates and prodrugs thereof;
(5′α)-2-Bromo-12′-hydroxy-5′-(2-methylpropyl)-3′,6′,18-trioxo-2′-(propan-2-yl)ergotaman (bromocriptine), disclosed in U.S. Pat. No. 3,752,814;
(6aR,9R,10aR)—N-[3-(dimethylamino)propyl]-N-(ethylcarbamoyl)-7-prop-2-enyl-6,6a,8,9,10,10a-hexahydro-4H-indolo[4,3-fg]quinoline-9-carboxamide (cabergoline), disclosed in U.S. Pat. No. 4,526,892;
2-{4-[(2S)-2-(3,4-dimethoxyphenyl)-2-hydroxyethyl]piperazin-1-yl}cyclohepta-2,4,6-trien-1-one (ciladopa), disclosed in EP 0034894;
5,6,6a,7,8,12b-hexahydro-benzo(a)phenanthridine-10,11-diol (dihydrexidine), disclosed in EP 0773933;
(2R,4R,7R)—N-[(1S,2S,4R,7S)-2-hydroxy-7-(2-methylpropyl)-5,8-dioxo-4-(propan-2-yl)-3-oxa-6,9-diazatricyclo[7.3.0.0^{2,6}]dodecan-4-yl]-6-methyl-6,11-diazatetracyclo[7.6.1.0^{2,7}.0^{12,16}]hexadeca-1(16),9,12,14-tetraene-4-carboxamide, (dihydroergocryptine); and its 7(S)isomer (epicriptine);
8,9-dihydroxy-2,3,7,11b-tetrahydro-1H-naph[1,2,3-de]isoquinoline (dinaspoline), disclosed in U.S. Pat. No. 5,047,536;
(+)-(6aS,12bR)-6a,7,8,12b-tetrahydro-6H-chromeno[3,4-c]isoquinoline-2,3-diol (doxanthrine), disclosed in US2009/0030025;
(2R,4R,7R)—N-[(1S,2S,4R,7S)-2-hydroxy-7-(S)-(1-methylpropyl)-5,8-dioxo-4-(propan-2-yl)-3-oxa-6,9-diazatricyclo[7.3.0.0^{2.6}]dodecan-4-yl]-6-methyl-6,11-diazatetracyclo[7.6.1.0^{2,7}.0^{12,16}]hexadeca-1(16),9,12,14-tetraene-4-carboxamide (epicriptine or beta-dihydroergocryptine) disclosed in U.S. Pat. No. 4,737,499;
1,1-Diethyl-3-(7-methyl-4,6,6a,7,8,9-hexahydro-indolo[4,3-fg]quinolin-9-yl)-urea (lisuride) disclosed in U.S. Pat. No. 3,953,454 and, in a transdermal therapeutic system, in U.S. Pat. No. 5,229,129;
(8β)-8-[(methylthio)methyl]-6-propylergoline (pergolide), disclosed in U.S. Pat. No. 4,166,182;
2-[4-(benzo[1,3]dioxol-5-ylmethyl)piperazin-1-yl]pyrimidine (piribedil), disclosed in U.S. Pat. No. 3,299,067;
(S)—N⁶-propyl-4,5,6,7-tetrahydro-1,3-benzothiazole-2,6-diamine, herein below referred to as (S)-6-propylamino-4,5,6,7-tetrahydro-1,3-benzothiazole-2-amine (pramipexole), disclosed in U.S. Pat. No. 4,886,812;
(6aS)-6-propyl-5,6,6a,7-tetrahydro-4H-dibenzo[de,g]quinoline-10,11-diol (propylnorapomorphine), disclosed in U.S. Pat. No. 3,717,643;
N,N-diethyl-N′-[(3S,4aS,10aR)-6-hydroxy-1-propyl-1,2,3,4,4a,5,10,10a-octahydrobenzo[g]quinolin-3-yl]sulfamide (quinagolide) disclosed in U.S. Pat. No. 4,565,818;
4-[(9,10-Didehydro-6-methylergolin-8β-yl)methyl]-2,6-piperazinedione (romergoline), disclosed in U.S. Pat. No. 4,728,649;
4-[2-(Dipropylamino)ethyl]-1,3-dihydro-2H-indol-2-one (ropinirole), disclosed in U.S. Pat. No. 4,452,808;
(S)-6-[propyl(2-thiophen-2-ylethyl)amino]-5,6,7,8-tetrahydronaphthalen-1-ol (rotigotine), disclosed in U.S. Pat. No. 6,372,920;
3-[4-(4-phenyl-3,6-dihydro-2H-pyridin-1-yl)butyl]-1H-indol-5-ol (roxindole), disclosed in U.S. Pat. No. 4,914,114 (as mesylate); and
(R)-5,6-Dihydro-5-(methylamino)-4H-imidazo[4,5,1-ij]quinolin-2(1H)-one
(sumanirole), obtainable as described by Romero A G, et al. “Synthesis of the selective D2 receptor agonist PNU-95666E from D-phenylalanine using a sequential oxidative cyclization strategy” (Journal of Organic Chemistry. 1997; 62(19):6582) and disclosed in U.S. Pat. No. 6,197,339 in an ER-formulation,
   (the contents of said patent documents being incorporated herein in their entirety by reference) and pharmaceutically acceptable salts and solvates and prodrugs thereof.

Illustrative examples of pharmaceutically acceptable salts or solvates of DA-agonist are derived from inorganic or organic acids such as hydrochloric acid, hydrobromic acid, sulfuric acid, sulfamic acid, phosphoric acid, nitric acid, acetic acid, propionic acid, stearic acid, glycolic acid, oxalic acid, succinic acid, lactic acid, maleic acid, hydroxymaleic acid, fumaric acid, malic acid, tartaric acid, citric acid, ascorbic acid, phenylacetic acid, glutamic acid, benzoic acid, salicylic acid, 2-acetoxybenzoic acid, methanesulfonic acid, ethanesulfonic acid, 2-hydroxyethanesulfonic (isethionic) acid, p-toluenesulfonic acid, 2-naphthalenesulfonic acid, 4-amino-benzenesulfonic (sulfanilic) acid, 2,6-naphthalenedisulfonic acid, 1,5-naphthalenedisulfonic acid, and pamoic (embonic) acid. The solvation solvent is normally water.

Some of the above DA-agonist are commercial drugs. In particular,
apomorphine is available in 10 mg/ml (30 mg/3 ml pen) for subcutaneous injection in cartridges each delivering single doses of 1 mg (herein below referred to as “unit dose”);
bromocriptine is available for example as Parlodel® (bromocriptine mesylate) in SnapTabs® scored tablets for oral administration, each containing 2.5 mg and in capsule containing 5 mg bromocriptine (as the mesylate), and is administered at a daily dose, including the titration phase, of from 2.5 mg to a maximum of 100 mg;
cabergoline is available for example as Dostinex® in 0.5 mg tablets and, in its indication for the treatment of hyperprolactinemic disorders, is administered at a daily dose, including the titration phase, of from 0.25 mg twice a week up to 1 mg twice a week;
dihydroergocryptine is available for example in the EU in unit forms comprising from 1 to 40 of mg dihydroergocryptine mesylate for oral use;
lisuride is available for example in EU as Dopergin® in 0.2 and 0.5 mg tablets, and in its indication for the treatment of PD, is administered at a daily dose, including the titration phase, of from 0.6 to 5 mg, divided in two-six, normally three single administrations;
pergolide mesylate is available for example as Nopar®, in 0,065 mg, 0,326 mg, and 1.3 mg tablets, equivalent to 0.05 mg, 0.25 mg, and, respectively, 1 mg pergolide base, and is administered at a daily dose, including the titration phase, of from 0.15 mg to the MRD of 3 mg;
piribedil, available for example in EU in IR-20 mg tablets and ER-50 mg tablets, to be administered in an adult patient at a daily dose of from 150 mg to 250 mg;
pramipexole is available for example as Mirapex® in tablets for immediate release containing 0.125 mg, 0.25 mg, 0.5 mg, 1 mg and 1.5 mg of pramipexole dihydrochloride monohydrate, and in tablets for extended release containing 4.5 mg of pramipexole dihydrochloride monohydrate, and is administered at a daily dose, including the titration phase, of from 0.375 mg to 4.5 mg;
quinagolide is available as Norprolac® (quinagolide hydrochloride) in strengths equivalent to 0.025 mg, 0.05 mg, 0.075 mg, and 0.15 mg of quinagolide base; and is administered once a day at a daily dose, including the titration phase, equivalent to from 0.025 mg to 0.9 mg or from 0.06 mg, normally from 0.025 mg to 0.3 mg of quinagolide base;
ropinirole is available as Requip® (ropinirole hydrochloride) in a strength equivalent to 0.25 mg, 0.5 mg, 1 mg, 2 mg, 3 mg, 4 mg, and 5 mg of ropinirole base, and is administered at a daily dose, including the titration phase, equivalent to from 0.25 mg to 24 mg of ropinirole base; and
rotigotine is available for example as Neupro®, .in three different dosages, in a transdermal patch delivering 2 mg/24 hours, 4 mg/24 hours, and, respectively, 6 mg/24 hours rotigotine.

According to the present invention, in the case of a studied or commercially available DA-agonist, the effective dose (daily or per unit form) of said DA-agonist to be used in combination with an AEsI for the safe treatment of PD and PD-related disorders, in particular MSA, DLB, LBD, RLS, DDS, FTLD, PSP, and CBD, is in a range shown effective or recommended by the Health Authorities but, in some case, the maximum (daily or per unit form) dose may be higher, and even much higher than said recommended or shown effective dose.

In fact, the use of an AEsI, as illustrated above in “The AEsI” section, allows for the full expression of the efficacy of the DA-agonists in the treatment of PD and PD-related disorders. In particular said use also allows the administration of daily or per unit form doses (of particular DA-agonists) that are higher, and even much higher than the maximum recommended or shown effective doses of said particular DA-agonists with a consequent increase of efficacy of said DA-agonist.

An advantageous DA-agonist according to the present invention is selected from the group consisting of apomorphine, bromocriptine, cabergoline, ciladopa, dihydrexidine, dihydroergocryptine, dinapsoline, doxanthrine, epicriptine, lisuride, pergolide, piribedil, pramipexole, propylnorapomorphine, quinagolide, romergoline, ropinirole, rotigotine, roxindole, sumanirole, and pharmaceutically acceptable salts and solvates and prodrugs of each of these DA-agonists.

For its administration in combination with an AEsI, the DA-agonist is formulated in a pharmaceutical composition in dosage unit form comprising, as an active ingredient, an effective dose per unit form of said DA-agonist in admixture with a pharmaceutical carrier or vehicle. Said active ingredient is formulated according to known technologies for any administration route.

In said pharmaceutical composition, the DA-agonist is present or is delivered in a dose per unit form of from 0.001 mg to 200 mg and is administered, in combination with an AEsI as described in the above “The AEsI-Component (a)” section to a patient suffering from a disease selected from the group consisting of PD and PD-related disorders (such as MSA, DLB, LBD, RLS, DDS, FTLD, PSP, and CBD), at a daily dose, including the titration phase, of from 0.025 mg to 1000 mg.

In particular, according to the present invention an advantageous DA-agonist is selected from the group consisting of apomorphine and pharmaceutically acceptable salts and solvates thereof, in an amount per unit dose equivalent to from 1 mg to 2 mg of apomorphine base, to be administered at a daily dose (in apomorphine base) of from 2 mg to 12 mg; bromocriptine and pharmaceutically acceptable salts and solvates thereof, in an amount per unit form equivalent to from 2.5 mg to 200 mg of bromocriptine base, to be administered at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, to be administered at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, to be administered at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, to be administered at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, to be administered at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; pramipexole, in an amount per unit form equivalent to from 0.125 mg to 45 mg of pramipexole dihydrochloride monohydrate, to be administered at a daily dose (in pramipexole dihydrochloride monohydrate) of from 0.375 mg to 45 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 75 mg, normally from 0.125 mg to 25 mg, from 0.25 mg to 20 mg or from 0.25 mg to 15 mg of ropinirole base, to be administered at a daily dose (in ropinirole base) of from 0.25 mg to 75 mg, normally from 0.125 mg to 15 mg, from 0.25 mg to 20 mg or from 0.25 mg to 25 mg; and rotigotine and pharmaceutically acceptable salts and solvates thereof, in an amount per unit form releasing from 2 mg to 24 mg of rotigotine base, to be administered at a daily dose equivalent to from 2 mg to 24 mg of rotigotine base.

A particularly advantageous DA-agonist in said unit form is selected from the group consisting of ciladopa, piribedil, pramipexole, quinagolide, ropinirole, rotigotine, roxindole, and sumanirole, and pharmaceutically acceptable salts and solvates and prodrugs of each of said DA-agonists each in the above respective amount per unit form and daily dose.

According to a preferred embodiment, the present invention provides a pharmaceutical composition in dosage unit form comprising, in admixture with a pharmaceutical carrier or vehicle, a DA-agonist selected from the group consisting of
piribedil and pharmaceutically acceptable salts and solvates and prodrugs thereof, such as its monomethanesulfonate salt, in an amount per unit form (in piribedil base) of from 20 mg to 200 mg, to be administered at a daily dose (in piribedil base) of from 150 to 1000 mg, normally from 300 mg to 600 mg;
pramipexole and pharmaceutically acceptable salts and solvates and prodrugs thereof, such as its dihydrochloride monohydrate, in an amount per unit form (in pramipexole dihydrochloride monohydrate) of from 0.125 mg to 45 mg, to be administered at a daily dose (in pramipexole dihydrochloride monohydrate) of from 0.375 mg to 45 mg;
quinagolide and pharmaceutically acceptable salts and solvates and prodrugs thereof, such as its hydrochloride salt in an amount per unit form equivalent to from 0.025 mg to 0.5 mg of quinagolide base, to be administered at a daily dose (in quinagolide base) of from 0.025 mg to 2 mg;
ropinirole and pharmaceutically acceptable salts and solvates and prodrugs thereof, such as its hydrochloride salt, in an amount per unit form (in ropinirole base) of from 0.25 mg to 75 mg, normally from 0.125 mg to 25 mg, from 0.25 mg to 20 mg or from 0.25 mg to 15 mg, to be administered at a daily dose (in ropinirole base) of from 0.25 mg to 75 mg, normally from 0.125 mg to 25 mg, from 0.25 mg to 20 mg or from 0.25 mg to 15 mg;
rotigotine and pharmaceutically acceptable salts and solvates and prodrugs thereof, such as its hydrochloride salt, in a TDDS delivering from 2 mg to 24 mg rotigotine, to be administered at a daily transdermal dose of from 2 mg to 24 mg, said administration of said DA-agonist to a patient suffering from PD or related disorders being made in combination with an AEsI.

According to an embodiment, in said pharmaceutical composition, the DA-agonist is pramipexole or pharmaceutically acceptable salt or solvate or prodrug thereof, in particular pramipexole dihydrochloride monohydrate. Stable pharmaceutical compositions comprising pramipexole dihydrochloride monohydrate are disclosed in WO 2012/0140604 and in WO 2008/122638, the contents of each of which are incorporated herein by reference in their entirety. Sustained release compositions comprising pramipexole dihydrochloride monohydrate are disclosed in U.S. Pat. No. 8,399,016, incorporated herein by reference in its entirety: These compositions may be for use in combination with an AEsI, in particular with a peripheral DA-antagonist, normally domperidone, with a 5HT3-antagonist and/or with a NK1-antagonist, for the treatment of a PMND such as PD and PD-related disorders.

According to the present invention, pramipexole is preferably used as pramipexole dihydrochloride monohydrate (US AN: pramipexole hydrochloride), in a dose per unit form of from 0.125 mg to 45 mg, or as free base, in a dose per unit form equivalent to from 0.125 mg to 45 mg of pramipexole dihydrochloride monohydrate.

As set forth in the above Summary of the Invention, the effective daily dose of pramipexole is a dose equivalent to at least the pramipexole dihydrochloride monohydrate approved daily dose. Said daily approved/recommended daily dose is from 0.125 mg to 4.5 mg, normally from 0.375 mg to 4.5 mg. However, it is hereby specified that, according to the present invention, the combination of an AEsI with said pramipexole or pharmaceutically acceptable salt thereof allows the administration of pramipexole dihydrochloride monohydrate approved/recommended daily doses and PD-related disorder without any adverse effect, but also allows the administration of pramipexole dihydrochloride monohydrate daily doses that are higher and also much higher than said approved doses.

In said combination with an AEsI, pramipexole or a pharmaceutically acceptable salt or solvate thereof, normally as pramipexole dihydrochloride monohydrate, may be administered to a patient suffering from a PD-related disorder selected from the group consisting of DDS, FTLD, PSP and CBD, at a daily dose (in pramipexole dihydrochloride monohydrate) of from 0.125 mg to 45 mg, normally from 0.375 mg to 45 mg, depending on the tolerability (in combination with the AEsI), and includes low doses to be administered during a titration period.

More particularly, in the treatment of a PD-related disorder selected from the group consisting of DDS, FTLD, PSP and CBD said daily dose range may be selected from the group consisting of form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg, depending on the tolerability (in combination with the AEsI). Said pramipexole daily dose normally is from 0.375 mg to 20 mg, from more than 4.5 mg to 20 mg, from more than 6 mg to 20 mg, from 10 mg to 20 mg, from 13 mg to 20 mg, from 14.5 mg to 45 mg, from 15 mg to 25 mg or from 15 mg to 20 mg of pramipexole dihydrochloride monohydrate, depending on the tolerability (in combination with the AEsI).

Specifically, pramipexole or a pharmaceutically acceptable salt or solvate thereof, normally as dihydrochloride monohydrate, may be administered to a patient suffering from RLS at a daily dose (in pramipexole dihydrochloride monohydrate) of from 0.125 mg to 6 mg or from 0.125 mg to 1 mg, normally from 0.125 mg to 0.75 mg or from 0.125 mg to 0.50 mg (in combination with the AEsI).

For its administration to a patient suffering from a PD-related disorder, in particular selected from the group consisting of RLS, DDS, FTLD, PSP, and CBD, in combination with an AEsI as illustrated above in “The AEsI” section, pramipexole is formulated in a pharmaceutical composition in dosage unit form comprising, as an active ingredient, said pramipexole in an effective amount per unit form, in admixture with a pharmaceutical carrier or vehicle.

According to the present invention, said pharmaceutical composition in dosage unit form comprises, as an active ingredient, pramipexole or a pharmaceutically acceptable salt thereof in an amount per unit form equivalent to from 0.125 mg to 30 mg, normally from 0.125 mg to 22.5 mg of pramipexole dihydrochloride monohydrate in an IR-formulation, or in an amount per unit form equivalent to from 0.375 mg to 45 mg, preferably from 1.5 mg to 40-42 mg of pramipexole dihydrochloride monohydrate in an ER-formulation.

In particular, the pramipexole amount (in pramipexole dihydrochloride monohydrate) in said unit form is in a range selected from the group consisting of from 0.125 mg to 45 mg, from 0.375 mg to 45 mg, form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg.

More particularly, said pramipexole is present in said composition
in an amount-range per IR-unit form equivalent to a pramipexole dihydrochloride monohydrate amount-range per unit form selected from the group consisting of 0.125 mg to 22.5 mg, from 0.75 mg to 22.5 mg, from 0.8 mg to 22.5 mg, from 1 mg to 22.5 mg, from 1.5 mg to 22.5 mg, from more than 2.25 mg to 2.5 mg, from 2.4 mg to 22.5 mg, from more than 3 mg to 22.5 mg, from 3.25 mg to 22.5 mg, from 5 mg to 22.5 mg, from 6.5 mg to 22.5 mg, from 7.25 mg to 22.5 mg; and from 7.5 mg to 22.5 mg, from more than 20 mg to 22.5 mg or from 20.25 mg to 25 mg, normally from 7.5 mg to 12.5 mg or from more than 10 mg to 12.5 mg; or
in an amount-range per ER-unit form equivalent to a pramipexole dihydrochloride monohydrate amount-range per unit form selected from the group consisting of from 0.375, mg to 45 mg, from 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 10 mg to 45 mg, from 13 mg to 45 mg, from 14.5 mg to 45 mg, and from 15 mg to 45 mg, normally from 15 mg to 25 mg or from more than 20 mg to 25 mg.

Especially, said pramipexole is present in said composition in an amount-range per unit form equivalent to a pramipexole dihydrochloride monohydrate amount-range per unit form selected from the group consisting of from 0.125 mg to 40-42 mg, from 0.125 mg to 30 mg, from 0.125 to 20 mg, from 1.5 mg to 40-42 mg, from 1.625 mg to 40-42 mg, from 3 mg to 40-42 mg, from more than 4.5 mg to 40-42 mg, from 4.8 mg to 40-42 mg, from more than 6 mg to 40-42 mg, from 10 mg to 40-42 mg from 13 mg to 40-42 mg, from 14.5 mg to 40-42 mg and from 15 mg to 40-42 mg.

Normally, said pramipexole is present in said composition in an amount-range per unit form equivalent to a pramipexole dihydrochloride monohydrate amount-range per unit 20 mg, from 1.625 mg to 20 mg, from 3 mg to 20 mg, from more than 4.5 mg to 20 mg, from 4.8 mg to 20 mg, from more than 6 mg to 20 mg, from 10 mg to 20 mg from 13 mg to 20 mg from 14.5 mg to 20 mg and from 15 mg to 20 mg.

In a preferred embodiment, the invention provides a pharmaceutical composition in dosage unit form comprising, as an active ingredient, pramipexole and pharmaceutically acceptable salts and solvates thereof, in an amount per unit form equivalent to from 13 mg to 45 mg, normally from 13 mg to 40-42 mg, from 13 mg to 30 mg or from 13 mg to 20 mg or from 14.5 mg to 40-42 mg of pramipexole dihydrochloride monohydrate, in admixture with a pharmaceutical carrier or vehicle.

In a specific embodiment, the invention provides a pharmaceutical composition in dosage unit form comprising, as an active ingredient, pramipexole and pharmaceutically acceptable salts and solvates thereof, in an amount per unit form equivalent to from 0.125 mg to 6 mg, administered at a daily dose (in pramipexole dihydrochloride monohydrate) of from 0.125 mg to 6 mg, for treatment of a patient suffering from RLS in combination with an AEsI, also in a pharmaceutical composition, in an amounts per unit form of from and daily dose as described in “The AEsI Component (a)” section above.

As set forth above, an AEsI, in combination with pramipexole or a pharmaceutically acceptable salt or solvate thereof, renders it possible to treat a patient suffering from RLS, DDS, FTLD, PSP, or CBD, by maintaining a therapeutically effective pramipexole or pharmaceutically acceptable salt or solvate thereof daily dose with minimal adverse effect.

Preferably, said AEsI in said combination is selected from the group consisting of
ondansetron and pharmaceutically acceptable salt and solvates and prodrugs thereof, in an amount per unit form equivalent to from 2 mg to 32 mg of ondansetron base, to be administered at a daily dose equivalent to from 6 mg to 64 mg, normally from 6 mg to 32 mg of ondansetron base;
dolasetron and pharmaceutically acceptable salts and solvates and prodrugs thereof, in an amount per unit form equivalent to 25 mg to 200 mg of dolasetron mesylate, to be administered at a daily dose equivalent to from 75 mg to 200 mg of dolasetron mesylate;
palonosetron and pharmaceutically acceptable salts and solvates and prodrugs thereof, in an amount per unit form equivalent to from 0.25 mg to 0.5 mg of palonosetron base, to be administered at a daily dose equivalent to from 0.75 to 2 mg of palonosetron base;
aprepitant and pharmaceutically acceptable salts and solvates and prodrugs thereof, in an amount per unit form and at a daily dose equivalent to from 10 mg to 250 mg of aprepitant;
fosaprepitant and pharmaceutically acceptable salts and solvates and prodrugs thereof, in an amount per unit form and at a daily dose equivalent to from 10 mg to 250 mg of aprepitant;
rolapitant and pharmaceutically acceptable salts and solvates and prodrugs thereof, in an amount per unit form and at a daily dose equivalent to from 15 mg to 270 mg of rolapitant,
netupitant and pharmaceutically acceptable salts and solvates and prodrugs thereof, in an amount per unit form and at a daily dose equivalent to from 300 mg to 600 mg;
netupitant-300/palonosetron-0.5;
fosnetupitant-235/palonosetron-0.25; and
domperidone and pharmaceutically acceptable salt and solvates and prodrugs thereof, in an amount per unit form equivalent to from 2 mg to 120 mg, normally from 2 mg to 40 mg, of domperidone base, to be administered at a daily dose equivalent to from 4 mg to 120 mg, normally from 4 mg to 40 mg, of domperidone base.

In order to provide concurrent administration of said AEsI and of said pramipexole or a pharmaceutically acceptable salt or solvate thereof, the invention provides a fixed-dose combination (ab) consisting of a pharmaceutical composition in dosage unit form comprising, as active ingredients, an AEsI, in an amount per unit form as illustrated in “The AEsI Component (a)” section, and pramipexole or a pharmaceutically acceptable salt or solvate thereof, in an amount per unit form as illustrated in this section, in admixture with a pharmaceutical carrier or vehicle.

Said fixed-dose combination is administered to a patient suffering from a PMND, in particular from PD or a PD-related disorder.

The AEsI and pramipexole fixed-dose combinations will be illustrated in “The fourth aspect of the invention” section below.

### First Aspect of the Invention

According to a first aspect, the present invention provides a method for safely improving the condition of a patient suffering from a PMND such as PD or a PD-related disorder and treated with a DA-agonist, by concurrently and chronically administering to said patients an AEsI.

More particularly, the invention provides a method for treating a disease selected from the group consisting of PD and PD-related disorders, in particular from a PD-related disorder selected from the group consisting of MSA, DLB, LBD, RLS, DDS, FTLD, PSP, and CBD, in a patient, which comprises administering to said patient in need of said treatment an effective daily dose of an AEsI in combination with an effective daily dose of a DA-agonist.

Preferably, said method is carried out by using an inhibitor of the adverse effects of DA-agonists for treating a Parkinson's disease related disorder selected from the group consisting of RLS, DDS, FTLD, PSP, and CBD in combination with a DA-agonist selected from the group consisting of pramipexole and pharmaceutically acceptable salts and solvates thereof.

As set forth in the respective sections, said effective daily dose of said AEsI is from 1 μg to 600 mg and said effective daily dose of the DA-agonist is from 0.025 mg to 1000 mg.

Any of the AEsIs illustrated in “The AEsI Component (a)” section may be used in a single dose (or in an unit form), in combination with any of the DA-agonists illustrated in “The DA-agonist Component (b)” section, to treat PD or a related disorder according to the method of this first aspect of the invention. Normally, said single dose of said AEsI is at least one selected from the group consisting of from 1 μg to 300 mg of a 5HT3-antagonist and from 1 μg to 600 mg of a NK1-antagonist, and from 2 mg to 120 mg, normally from 2 mg to 40 mg of domperidone, said single dose being administered to a patient one or more times per day.

In said combination, the AEsI is used in a pharmaceutical composition in dosage unit form comprising, as an active ingredient, said AEsI, in an amount per unit form of from 1 μg to 600 mg, in admixture with a pharmaceutical carrier or vehicle, and is administered at a daily dose of from 1 μg to 600 mg. This administration is made in combination with a DA-agonist, also in a pharmaceutical composition in dosage unit form comprising said DA-agonist in an amount per unit form of from 0.001 mg to 200 mg, concurrently or sequentially administered at a daily dose of from 0.5 mg to 1000 mg for the treatment of PD and PD-related disorders.

Thus, for example, a pharmaceutical composition according to the present invention to be administered in combination with a DA-agonist, may comprise at least one AEsI selected from the group consisting of

(A) a 5HT3-antagonist selected from the group consisting of azasetron and pharmaceutically acceptable salts and solvates thereof, in an amount per unit form equivalent to from 5 mg to 10 mg of azasetron hydrochloride, to be administered at a daily dose equivalent to from 15 mg to 40 mg of azasetron hydrochloride; dolasetron and pharmaceutically acceptable salts and solvates thereof, in an amount per unit form equivalent to from 25 mg to 200 mg of dolasetron mesylate, to be administered at a daily dose equivalent to from 75 mg to 200 mg of dolasetron mesylate; granisetron and pharmaceutically acceptable salts and solvates thereof, in an amount per unit form equivalent to from 0.5 mg to 2 mg granisetron base, to be administered at a daily dose equivalent to from 1.5 mg to 8 mg of granisetron base; ondansetron and pharmaceutically acceptable salts and solvates thereof, in an amount per unit form equivalent to from 0.5 mg to 16 mg, normally from 2 mg to 8 mg of ondansetron base, to be administered at a daily dose equivalent to from 6 mg to 64 mg, normally from 6 mg to 32 mg of ondansetron base; palonosetron and pharmaceutically acceptable salts and solvates thereof, in an amount per unit form equivalent to from 0.25 mg to 0.5 mg of palonosetron base, to be administered at a daily dose equivalent to from 0.75 to 2 mg of palonosetron base; ramosetron and pharmaceutically acceptable salts and solvates thereof, in an amount per unit form equivalent to from 0.05 mg to 0.2 mg of ramosetron hydrochloride, to be administered at a daily dose equivalent to from 0.05 mg to 0.2 mg of ramosetron hydrochloride; and tropisetron and pharmaceutically acceptable salts and solvates thereof, in an amount per unit form equivalent to from 2.5 mg to 5 mg of tropisetron base, to be administered at a daily dose equivalent to from 7.5 mg to 20 mg of tropisetron base;

(B) a NK1-antagonist selected from the group consisting of aprepitant and pharmaceutically acceptable salts and solvates thereof, in an amount per unit form and at a daily dose equivalent to from 10 mg to 250 mg of aprepitant; fosaprepitant and pharmaceutically acceptable salts and solvates thereof, in an amount per unit form and at a daily dose equivalent to from 10 mg to 250 mg of aprepitant; rolapitant and pharmaceutically acceptable salts and solvates thereof, in an amount per unit form and at a daily dose equivalent to from 15 mg to 270 mg of rolapitant, netupitant and pharmaceutically acceptable salts and solvates thereof, in an amount per unit form and at a daily dose equivalent to from 300 mg to 600 mg; netupitant-300/palonosetron-0.5 once a day; and fosnetupitant-235/palonosetron-0.25 once a day; and

(C) a peripheral DA-antagonist selected from the group consisting of domperidone and pharmaceutically acceptable salt and solvates thereof, in an amount per unit form (in domperidone base) of from 2 mg to 120 mg and at a daily dose (in domperidone base) of from 4 mg to 120 mg.

For the treatment of a disease selected from the group consisting of a PMND such as PD and PD-related disorders, the at least one AEsI, in a pharmaceutical composition comprising said at least one AEsI, in an amount per unit form of from 1 μg to 600 mg, is administered at a daily dose of from 1 μg to 600 mg in combination with a DA-agonist, also in a pharmaceutical composition comprising, as an active ingredient, said DA-agonist in an amount per unit form of from 0.001 mg to 200 mg, administered at a daily dose of from 0.025 mg to 1000 mg.

According to a first embodiment, said pharmaceutical composition comprising said at least one AEI is administered to a patient suffering from a PMND such as PD or a PD-related disorder, in combination with a DA-agonist selected from the group consisting of apomorphine, bromocriptine, cabergoline, ciladopa, dihydrexidine, dihydroergocryptine, dinapsoline doxanthrine, epicriptine, lisuride, pergolide, piribedil, propylnorapomorphine, quinagolide, romergoline, ropinirole, rotigotine, roxindole, sumanirole, and pharmaceutically acceptable salts and solvates and prodrugs thereof.

In particular, according to this first embodiment, in combination with the above composition comprising said at least one AEsI, said DA-agonist, also in a pharmaceutical composition, is selected from the group consisting of apomorphine and pharmaceutically acceptable salts and solvates thereof, in an amount per unit dose equivalent to from 1 mg to 2 mg of apomorphine base, to be administered at a daily dose (in apomorphine base) of from 2 mg to 12 mg; bromocriptine and pharmaceutically acceptable salts and solvates thereof, in an amount per unit form equivalent to from 2.5 mg to 200 mg of bromocriptine base, to be administered at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, to be administered at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, to be administered at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, to be administered at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, to be administered at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 75 mg of ropinirole base, to be administered at a daily dose (in ropinirole base) of from 0.25 mg to 75 mg; and rotigotine and pharmaceutically acceptable salts and solvates thereof, in an amount per unit form releasing from 2 mg to 24 mg of rotigotine base, to be administered at a daily dose equivalent to from 2 mg to 24 mg of rotigotine base; for the treatment of a patient suffering from a PMND selected from the group consisting of PD and PD-related disorders, in particular from a PD-related disorder selected from the group consisting of MSA, DLB, LBD, RLS, DDS, FTLD, PSP, and CBD.

Preferably, in said composition comprising said at least one AEsI, said AEsI is selected from the group consisting of ondansetron and pharmaceutically acceptable salt and solvates and prodrugs thereof, in an amount per unit form equivalent to from 2 mg to 32 mg of ondansetron base, to be administered at a daily dose equivalent to from 6 mg to 64 mg, normally from 6 mg to 32 mg of ondansetron base; dolasetron and pharmaceutically acceptable salts and solvates and prodrugs thereof, in an amount per unit form equivalent to 25 mg to 200 mg of dolasetron mesylate, to be administered at a daily dose equivalent to from 75 mg to 200 mg of dolasetron mesylate; palonosetron and pharmaceutically acceptable salts and solvates and prodrugs thereof, in an amount per unit form equivalent to from 0.25 mg to 0.5 mg of palonosetron base, to be administered at a daily dose equivalent to from 0.75 to 2 mg of palonosetron base; aprepitant and pharmaceutically acceptable salts and solvates and prodrugs thereof, in an amount per unit form and at a daily dose equivalent to from 10 mg to 250 mg of aprepitant; fosaprepitant and pharmaceutically acceptable salts and solvates and prodrugs thereof, in an amount per unit form and at a daily dose equivalent to from 10 mg to 250 mg of aprepitant; rolapitant and pharmaceutically acceptable salts and solvates and prodrugs thereof, in an amount per unit form and at a daily dose equivalent to from 15 mg to 270 mg of rolapitant; netupitant and pharmaceutically acceptable salts and solvates and prodrugs thereof, in an amount per unit form and at a daily dose equivalent to from 300 mg to 600 mg; netupitant-300/palonosetron-0.5; and fosnetupitant-235/palonosetron-0.25 once a day

According to a second embodiment, said at least one AEsI in said composition is selected from the group consisting of domperidone and pharmaceutically acceptable salt and solvates thereof, in an amount per unit form equivalent to from 2 mg to 120 mg, normally from 2 mg to 40 mg of domperidone base, to be administered at a daily dose equivalent to from 4 mg to 120 mg, normally from 4 mg to 40 mg of domperidone base, to be administered to a patient suffering from a PMND in combination with a DA-agonist selected from the group consisting of apomorphine, bromocriptine, cabergoline, ciladopa dihydrexidine, dihydrexidine, dihydroergocryptine, dinapsoline doxanthrine, lisuride, pergolide, piribedil, pramipexole, propylnorapomorphine, quinagolide, romergoline, ropinirole, rotigotine, roxindole and sumanirole, each also in a pharmaceutical composition in dosage unit form comprising, as an active ingredient, said DA-agonist in an amount per unit form of from 0.001 mg to 200 mg, in admixture with a pharmaceutical carrier or vehicle.

In particular, according to this second embodiment, in combination with domperidone, said DA-agonist is selected from the group consisting of apomorphine and pharmaceutically acceptable salts and solvates thereof, in an amount per unit dose equivalent to from 1 mg to 2 mg of apomorphine base; bromocriptine and pharmaceutically acceptable salts and solvates thereof, in an amount per unit form equivalent to from 2.5 mg to 200 mg of bromocriptine base, at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; pramipexole, in an amount per unit form equivalent to from 0.125 mg to 45 mg of pramipexole dihydrochloride monohydrate, to be administered at a daily dose (in pramipexole dihydrochloride monohydrate) of from 0.375 mg to 45 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 20 mg of ropinirole hydrochloride, to be administered at a daily dose (in ropinirole hydrochloride) of from 0.25 mg to 20 mg; and rotigotine and pharmaceutically acceptable salts and solvates thereof, in an amount per unit form releasing from 2 mg to 24 mg of rotigotine base, to be administered at a daily dose equivalent to from 2 mg to 24 mg of rotigotine base.

Said pharmaceutical composition comprising said at least one AEsI and said pharmaceutical composition comprising said DA-agonist thus obtained are concurrently or sequentially administered to a patient suffering from a PMND such as PD and PD-related disorders, in particular MSA, DLB, LBD, RLS, DDS, FTLD, PSP, and CBD.

According to a third embodiment, said method is for the treatment of a patient suffering from of a PMND consisting of a PD-related disorder selected from the group consisting of DDS, FTLD, PSP, and CBD, with an effective daily dose of at least one AEsI, formulated in a pharmaceutical composition comprising said AEsI, selected from group consisting of ondansetron and pharmaceutically acceptable salt and solvates and prodrugs thereof, in an amount per unit form equivalent to from 2 mg to 32 mg of ondansetron base, and dolasetron and pharmaceutically acceptable salts and solvates and prodrugs thereof, in an amount per unit form equivalent to 25 mg to 200 mg of dolasetron mesylate; aprepitant and pharmaceutically acceptable salt and solvates and prodrugs thereof, in an amount per unit form equivalent to from 10 mg to 250 mg of aprepitant base, and rolapitant and pharmaceutically acceptable salts and solvates and prodrugs thereof, in an amount per unit form equivalent to 15 mg to 270 mg of rolapitant base; in combination with a DA-agonist selected from the group consisting pramipexole and pharmaceutically acceptable salts and solvates thereof, in an amount per unit form equivalent to from 0.125 mg to 45 mg of pramipexole dihydrochloride monohydrate, to be administered at a daily dose equivalent to from 0.375 mg to 45 mg, normally from 1.5 mg to 45 mg, preferably from more than 4.5 mg to 45 mg, normally from 15 mg to 25 mg or from more than 20 mg to 25 mg of pramipexole dihydrochloride monohydrate.

According to the above second and third embodiment of this first aspect of the invention, the pramipexole amounts per unit form and daily doses are those described in “The DA-agonist Component (b)” section. Specifically, in the second and third embodiments of this section, the pramipexole amount (in pramipexole dihydrochloride monohydrate) per unit form is in a range selected from the group consisting of from 0.125 mg to 45 mg, from 0.375 mg to 45 mg, form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg. Normally said range is from 15 mg to 25 mg or from more than 20 mg to 25 mg.

For its normal use and in particular at the beginning of the treatment, said pramipexole or pharmaceutically acceptable salt or solvate thereof Component (b) is preferably present in said composition in an amount per unit form equivalent to from 0.125 mg to 20 mg, from 1.6 mg to 20 mg, from 1.625 mg to 20 mg, from 3 mg to 20 mg, from more than 4.5 mg to 20 mg, from more than 6 mg to 20 mg, from 6.5 mg to 20 mg, from 7.25 mg to 20 mg, from 7.5 mg to 20 mg, from 10 mg to 20 mg, from 13 mg to 20 mg, from 14.5 mg to 20 mg, and from 15 mg to 20 mg, normally from 1.5 mg to 20 mg, of pramipexole dihydrochloride monohydrate.

For the administration of pramipexole at higher doses, said pramipexole may be present in said composition in an amount equivalent to a wider range selected from the group consisting of from more than 1.5 mg to 45 mg, from 1.6 mg to 45 mg, from 1.625 mg to 45 mg, from 3 mg to 45 mg, from more than 4.5 mg to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg, and from 15 mg to 45 mg of pramipexole dihydrochloride monohydrate. As set forth above, said range normally is from 15 mg to 25 mg or from more than 20 mg to 25 mg.

In said combination, including fixed-dose combinations, said pramipexole or pharmaceutically acceptable salt or solvate thereof is normally present, in said pharmaceutical composition, in an amount per unit form equivalent to from 0.125 mg to 10 mg up to from 7.5 mg to 10 mg of pramipexole dihydrochloride monohydrate, in admixture with a pharmaceutical carrier or vehicle for an immediate-release formulation. For the administration of pramipexole at higher doses, said amount per IR-unit form will be equivalent to from 1.5 mg to 22.5 mg, from 1.6 mg to 22.5 mg, from more than 4.5 mg to 22.5 or from 7.5 mg to 22.5 mg, normally from 1.5 mg to 20 mg, of pramipexole dihydrochloride monohydrate.

Said pramipexole or pharmaceutically acceptable salt or solvate thereof will be present, in said pharmaceutical composition, in an amount per unit form equivalent to 1.5 mg to 45 mg or from more than 4.5 mg to 20 mg up to from 15 mg to 20 mg, of pramipexole dihydrochloride monohydrate, in admixture with a pharmaceutical carrier or vehicle for an extended-release formulation. For the administration of pramipexole at higher doses, said amount per ER-unit form will be equivalent to from more than 4.5 mg to 45 mg, up to or from 15 mg to 45 mg, normally from 15 mg to 25 mg or from more than 20 mg to 25 mg of pramipexole dihydrochloride monohydrate.

In particular, in the method (or use) for treating DDS, FTLD, PSP, or CBD in a patient according to the present invention, the AEsI/DA-agonist combination is selected from the group consisting of
ondansetron hydrochloride dihydrate, in an amount per unit form (in ondansetron base) of from 2 mg to 32 mg, at an effective daily dose (in ondansetron base) of from 4 mg to 32 mg and pramipexole dihydrochloride monohydrate, in an amount per unit form (in pramipexole dihydrochloride monohydrate) of from 0.125 mg to 45 mg, normally or from 15 mg to 25 mg or from more than 20 mg to 25 mg, and at an effective daily dose of from 1.5 mg to 45 mg, normally from 15 mg to 25 mg or from more than 20 mg to 25 mg; and
domperidone base, in an amount per unit form (in domperidone base) of from 2 mg to 120 mg or from 2 mg to 40 mg, at an effective daily dose of from 4 mg to 120 mg, normally from 4 mg to 40 mg and pramipexole dihydrochloride monohydrate, in an amount per unit form (in pramipexole dihydrochloride monohydrate) of from 0.125 mg to 45 mg, normally from 15 mg to 25 mg or from more than 20 mg to 25 mg, and at an effective daily dose of from 1.5 mg to 45 mg, normally from 15 mg to 25 mg or from more than 20 mg to 25 mg.

Preferably, the present invention provides a method for treating PD in a patient, which comprises administering to said patient a peripheral DA-antagonist selected from the group consisting of domperidone and pharmaceutically acceptable salt and solvates thereof, in combination with a DA-agonist selected from the group consisting of pramipexole and pharmaceutically acceptable salts and solvates thereof, at a daily dose equivalent to from 0.125 mg to 45 mg, normally from 0.375 mg to 45 mg, of pramipexole dihydrochloride monohydrate.

Said domperidone DA-antagonist is concurrently or sequentially administered to said patient, in a pharmaceutical composition comprising domperidone or a pharmaceutically acceptable salt or solvate thereof in an amount per unit form equivalent to from 2 mg to 120 mg, normally from 2 mg to 40 mg, of domperidone base, at a daily dose equivalent to from 4 mg to 120 mg, normally from 4 mg to 40 mg, of domperidone base, in combination with said pramipexole DA-agonist, also in a pharmaceutical composition comprising pramipexole or a pharmaceutically acceptable salt or solvate thereof in an amount per unit form equivalent to from 0.125 mg to 45 mg of pramipexole dihydrochloride monohydrate, at the aforementioned daily dose.

According to a fourth embodiment, the invention provides a method for treating RLS in a patient in a patient in need of said treatment, which comprises administering to said patient at least one AEsI selected from the group consisting of domperidone and pharmaceutically acceptable salts and solvates thereof, in an amount per unit form equivalent to from 2 mg to 40 mg of domperidone base, to be administered at a daily dose equivalent to fro 4 mg to 40 mg of domperidone base, ondansetron and pharmaceutically acceptable salt and solvates and prodrugs thereof, in an amount per unit form equivalent to from 2 mg to 32 mg of ondansetron base, normally from 2 mg to 16 mg or from 2 mg to 8 mg, to be administered at a daily dose equivalent to from 6 mg to 32 mg, normally from 6 mg to 16 mg or from 2 mg to 8 mg of ondansetron base; dolasetron and pharmaceutically acceptable salts and solvates and prodrugs thereof, in an amount per unit form equivalent to 25 mg to 200 mg, normally from 25 mg to 100 mg of dolasetron mesylate, to be administered at a daily dose equivalent to from 75 mg to 200 mg, normally from 75 mg to 100 of dolasetron mesylate; palonosetron and pharmaceutically acceptable salts and solvates and prodrugs thereof, in an amount per unit form equivalent to from 0.25 mg to 0.5 mg, normally from 0.25 mg to 0.25 mg of palonosetron base, to be administered at a daily dose equivalent to from 0.75 to 2 mg, normally from 0.75 mg to 1 mg of palonosetron base; aprepitant and pharmaceutically acceptable salts and solvates and prodrugs thereof, in an amount per unit form and at a daily dose equivalent to from 10 mg to 250 mg, normally from 10 mg to 125 mg of aprepitant; fosaprepitant and pharmaceutically acceptable salts and solvates and prodrugs thereof, in an amount per unit form and at a daily dose equivalent to from 10 mg to 250 mg, normally from 10 mg to 125 mg of aprepitant; rolapitant and pharmaceutically acceptable salts and solvates and prodrugs thereof, in an amount per unit form and at a daily dose equivalent to from 15 mg to 270 mg, normally from 15 mg to 135 mg of rolapitant; netupitant and pharmaceutically acceptable salts and solvates and prodrugs thereof, in an amount per unit form and at a daily dose equivalent to from 300 mg to 600 mg, normally of 300 mg; netupitant-300/palonosetron-0.5; and fosnetupitant-235/palonosetron-0.25, in combination with a DA-agonist selected from the group consisting of pramipexole and pharmaceutically acceptable salts and solvates thereof, in an amount per unit form equivalent to from 0.125 mg to 6 mg, from 0.125 mg to 1 mg, from 0.125 mg to 0.75 mg, and from 0.125 mg to 0.5 mg of pramipexole dihydrochloride monohydrate, to be administered at a daily dose equivalent to from 0.125 mg to 6 mg, from 0.125 mg to 1 mg, from 0.125 mg to 0.75 mg, and from 0.125 mg to 0.5 mg of pramipexole dihydrochloride monohydrate.

In particular, according to this fourth embodiment of this first aspect of the invention, in the treatment of a patient suffering from RLS, the AEsI/DA-agonist combination comprises or consists of at least one AEsI selected from the group consisting of domperidone base, domperidone maleate and domperidone succinate (1:1), in an amount per unit form equivalent to from 2 mg to 40 mg of domperidone base, administered at an effective daily dose equivalent to from 4 mg to 40 mg of domperidone base in combination, including fixed-dose combinations, with a DA-agonist selected from the group consisting of pramipexole base and pramipexole dihydrochloride monohydrate, at an effective daily dose equivalent to from 0.125 mg to 6 mg, from 0.125 mg to 1 mg, from 0.125 mg to 0.75 mg, or from 0.125 mg to 0.5 mg of pramipexole dihydrochloride monohydrate. Preferably, said fixed-dose combination comprises or consists of a pharmaceutical composition comprising a pharmaceutical carrier or vehicle and a fixed-dose combination of domperidone, in an amount (in domperidone base) of from 2 mg to 40 mg, and pramipexole, in an amount (in pramipexole dihydrochloride monohydrate) of from 0.125 mg to 6 mg, normally from 0.125 mg to 1 mg.

According to this fourth embodiment, the invention also provides a pharmaceutical composition in dosage unit form comprising ondansetron or a pharmaceutically acceptable salt or solvates or prodrugs thereof, in an amount per unit form equivalent to from 2 mg to 32 mg, normally from 2 mg to 16 mg or from 2 mg to 8 mg of ondansetron base, in admixture with a pharmaceutical carrier or vehicle, for use for the treatment of SRL in a patient in need of said treatment, in combination with pramipexole or a pharmaceutically acceptable salt or solvate or prodrug thereof at a daily dose of from 0.125 mg to 6 mg, normally from 0.125 mg to 1 mg.

### Second Aspect of the Invention

According to a second aspect, the invention provides an AEsI Component (a) for use for the treatment of a PMND in combination, including fixed dose combinations) with a DA-agonist Component (b). Said use is effective in the treatment of a PMND especially selected from the group consisting of PD and PD-related disorders, a PD-related disorder being in particular MSA, DLB, LDB, RLS, DDS, FTLD, PSP, or CBD, in a patient in need of said treatment.

In particular, this second aspect of the present invention provides an AEsI, in an amount per unit form of from 1 μg to 600 mg and at a daily dose of from 0.1 μg to 600 mg, for use in combination with a daily dose of said DA-agonist of from 0.025 mg to 1000 mg, for the treatment of PD and PD-related disorders in a patient in need of said treatment. Said effective daily dose of said DA-agonist may be higher, and even much higher than the maximum recommended dose used in the treatment of PD.

Normally, said AEsI for said use is selected among the (A)-(C)AEsIs, as illustrated above in “The AEsI Component (a)” section and in the disclosure of first aspect of the invention and is formulated in a pharmaceutical composition in dosage unit form comprising said AEsI in an effective amount per unit form.

Any of the AEsIs illustrated in “The AEsI Component (a)” section may be used in a single dose (or in an unit form), in combination with any of the DA-agonists illustrated in “The DA-agonist Component (b)” section, to treat a PMND according to this second aspect of the invention. Normally, said single dose of said AEsI is from 1 μg to 600 mg, to be administered to a patient one or more times per day. This administration is made in combination with a DA-agonist, also in a pharmaceutical composition in dosage unit form comprising said DA-agonist in an amount per unit form of from 0.001 mg to 200 mg, concurrently or sequentially administered at a daily dose of from 0.5 mg to 1000 mg for the treatment of a PMND such as PD or a PD-related disorder.

A pharmaceutical composition according to this second aspect of the invention, to be administered in combination with a DA-agonist, may comprise at least one AEsI selected from the group consisting of

(A) a 5HT3-antagonist selected from the group consisting of azasetron and pharmaceutically acceptable salts and solvates thereof, in an amount per unit form equivalent to from 5 mg to 10 mg of azasetron hydrochloride, to be administered at a daily dose equivalent to from 15 mg to 40 mg of azasetron hydrochloride; dolasetron and pharmaceutically acceptable salts and solvates thereof, in an amount per unit form equivalent to from 25 mg to 200 mg of dolasetron mesylate, to be administered at a daily dose equivalent to from 75 mg to 200 mg of dolasetron mesylate; granisetron and pharmaceutically acceptable salts and solvates thereof, in an amount per unit form equivalent to from 0.5 mg to 2 mg granisetron base, to be administered at a daily dose equivalent to from 1.5 mg to 8 mg of granisetron base; ondansetron and pharmaceutically acceptable salts and solvates thereof, in an amount per unit form equivalent to from 0.5 mg to 16 mg, normally from 2 mg to 8 mg of ondansetron base, to be administered at a daily dose equivalent to from 6 mg to 64 mg, normally from 6 mg to 32 mg of ondansetron base; palonosetron and pharmaceutically acceptable salts and solvates thereof, in an amount per unit form equivalent to from 0.25 mg to 0.5 mg of palonosetron base, to be administered at a daily dose equivalent to from 0.75 to 2 mg of palonosetron base; ramosetron and pharmaceutically acceptable salts and solvates thereof, in an amount per unit form equivalent to from 0.05 mg to 0.2 mg of ramosetron hydrochloride, to be administered at a daily dose equivalent to from 0.05 mg to 0.2 mg of ramosetron hydrochloride; and tropisetron and pharmaceutically acceptable salts and solvates thereof, in an amount per unit form equivalent to from 2.5 mg to 5 mg of tropisetron base, to be administered at a daily dose equivalent to from 7.5 mg to 20 mg of tropisetron base; and

(B) a NK1-antagonist selected from the group consisting of aprepitant and pharmaceutically acceptable salts and solvates thereof, in an amount per unit form and at a daily dose equivalent to from 10 mg to 250 mg of aprepitant; fosaprepitant and pharmaceutically acceptable salts and solvates thereof, in an amount per unit form and at a daily dose equivalent to from 10 mg to 250 mg of aprepitant; rolapitant and pharmaceutically acceptable salts and solvates thereof, in an amount per unit form and at a daily dose equivalent to from 15 mg to 270 mg of rolapitant, netupitant and pharmaceutically acceptable salts and solvates thereof, in an amount per unit form and at a daily dose equivalent to from 300 mg to 600 mg; netupitant-300/palonosetron-0.5 once a day; and fosnetupitant-235/palonosetron-0.25 once a day; and

(C) a peripheral DA-antagonist selected from the group consisting of domperidone and pharmaceutically acceptable salts and solvates thereof, in an amount per unit form of from 2 mg to 120 mg of domperidone base and at a daily dose of from 4 mg to 120 mg, normally from 4 mg to 40 mg of domperidone base.

Each of the above AEsI are administered at the respective daily dose for the treatment of a PMND preferably selected from the group consisting of PD and PD-related disorders in combination with a DA-agonist, administered to said patient at a daily dose of from 0.025 mg 1000 mg.

According to a first embodiment of this second aspect of the invention, said pharmaceutical composition comprising said AEI is administered to a patient suffering from a PMND such as AD, PD or a PD-related disorder, in combination with a DA-agonist selected from the group consisting of apomorphine, bromocriptine, cabergoline, ciladopa dihydrexidine, dihydrexidine, dihydroergocryptine, dinapsoline doxanthrine, lisuride, pergolide, piribedil, propylnorapomorphine, quinagolide, romergoline, ropinirole, rotigotine, roxindole and sumanirole, each also in a pharmaceutical composition in dosage unit form comprising, as an active ingredient, said DA-agonist in an amount per unit form of from 0.001 mg to 200 mg, in admixture with a pharmaceutical carrier or vehicle.

In particular, according to this first embodiment, for the use according to the present invention for treating a PMND selected from the group consisting of AD, PD and PD-related disorders,

For this use, said at least one AEsI in said composition is selected from the group consisting of domperidone and pharmaceutically acceptable salts and solvates thereof, in an amount per unit form equivalent to from 2 mg to 120 mg, normally from 2 mg to 40 mg of domperidone base, to be administered at a daily dose equivalent to from 4 mg to 120 mg, normally from 4 mg to 40 mg of ondansetron base; ondansetron and pharmaceutically acceptable salt and solvates and prodrugs thereof, in an amount per unit form equivalent to from 2 mg to 32 mg of ondansetron base, to be administered at a daily dose equivalent to from 6 mg to 64 mg, normally from 6 mg to 32 mg of ondansetron base; dolasetron and pharmaceutically acceptable salts and solvates and prodrugs thereof, in an amount per unit form equivalent to 25 mg to 200 mg of dolasetron mesylate, to be administered at a daily dose equivalent to from 75 mg to 200 mg of dolasetron mesylate; palonosetron and pharmaceutically acceptable salts and solvates and prodrugs thereof, in an amount per unit form equivalent to from 0.25 mg to 0.5 mg of palonosetron base, to be administered at a daily dose equivalent to from 0.75 to 2 mg of palonosetron base; aprepitant and pharmaceutically acceptable salts and solvates and prodrugs thereof, in an amount per unit form and at a daily dose equivalent to from 10 mg to 250 mg of aprepitant; fosaprepitant and pharmaceutically acceptable salts and solvates and prodrugs thereof, in an amount per unit form and at a daily dose equivalent to from 10 mg to 250 mg of aprepitant; rolapitant and pharmaceutically acceptable salts and solvates and prodrugs thereof, in an amount per unit form and at a daily dose equivalent to from 15 mg to 270 mg of rolapitant; netupitant and pharmaceutically acceptable salts and solvates and prodrugs thereof, in an amount per unit form and at a daily dose equivalent to from 300 mg to 600 mg; netupitant-300/palonosetron-0.5; and fosnetupitant-235/palonosetron-0.25 once a day

These Adverse Effect Inhibitors, each in the above amount per unit form and daily dose may be administered to a patient, in combination with a pharmaceutical composition comprising a pharmaceutical carrier or vehicle and a DA-agonist selected from the group consisting of apomorphine and pharmaceutically acceptable salts and solvates thereof, in an amount per unit dose equivalent to from 1 mg to 2 mg of apomorphine base, to be administered at a daily dose (in apomorphine base) of from 2 mg to 12 mg; bromocriptine and pharmaceutically acceptable salts and solvates thereof, in an amount per unit form equivalent to from 2.5 mg to 200 mg of bromocriptine base, to be administered at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, to be administered at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, to be administered at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, to be administered at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, to be administered at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; quinagolide and pharmaceutically acceptable salts and solvates thereof, in an amount per unit form equivalent to from 0.025 mg to 0.5 mg of quinagolide base, at a daily dose (in quinagolide base) of from 0.025 mg to 0.9 mg; ropinirole and pharmaceutically acceptable salts and solvates thereof, in an amount per unit form equivalent to from 0.25 mg to 75 mg, normally from 0.125 mg to 25 mg, from 0.25 mg to 20 mg or from 0.25 mg to 15 mg of ropinirole base, administered at a daily dose (in ropinirole base) of from 0.25 mg to 75 mg, normally from 0.125 mg to 25 mg, from 0.25 mg to 20 mg or from 0.25 mg to 15 mg; and rotigotine and pharmaceutically acceptable salts and solvates thereof, in an amount per unit form releasing from 2 mg to 24 mg of rotigotine base, to be administered at a daily dose equivalent to from 2 mg to 24 mg of rotigotine base.

Preferably, said DA-agonist is a DA-agonist selected from the group consisting of piribedil and pharmaceutically acceptable salts and solvate and prodrugs thereof, administered at a daily dose (in piribedil base) of from 150 mg to 1000 mg; quinagolide and pharmaceutically acceptable salts and solvates and prodrugs thereof, administered at a daily dose (in quinagolide base) of from 0.025 mg to 0.9 mg; ropinirole and pharmaceutically acceptable salts and solvates and prodrugs thereof administered at a daily dose (in ropinirole hydrochloride) of from 0.25 mg to 75 mg; and rotigotine and pharmaceutically acceptable salts and solvates thereof, at a daily dose equivalent to, from 2 mg to 24 mg of rotigotine base.

Said daily dose of said DA-agonists are provided by administering said piribedil and pharmaceutically acceptable salts and solvates and prodrugs thereof, in an amount per unit form (in piribedil base) of from 20 mg to 200 mg; said quinagolide, in an amount per unit form (in quinagolide base) of from 0.025 mg to 0.5 mg, said ropinirole, in an amount (in ropinirole base) per unit form of from 0.25 mg to 75 mg; and said rotigotine and pharmaceutically acceptable salts and solvates thereof, in an amount (in rotigotine base) per unit form releasing from 2 mg to 24 mg of rotigotine base.

Said pharmaceutical composition comprising said AEsI and said pharmaceutical composition comprising said DA-agonist thus obtained are concurrently or sequentially administered to a patient suffering from a PMND, especially from PD or a PD-related disorder, said PD-related disorder being in particular selected from the group consisting of MSA, DLB, LBD, RLS, DDS, FTLD, PSP, and CBD.

According to a second embodiment, the invention provides at least one AEsI selected from the group consisting of domperidone and pharmaceutically acceptable salts thereof for use in the treatment of a PMND, in combination with a DA-agonist selected from the group consisting of apomorphine, bromocriptine, cabergoline, ciladopa dihydrexidine, dihydrexidine, dihydroergocryptine, dinapsoline doxanthrine, lisuride, pergolide, piribedil, pramipexole, propylnorapomorphine, quinagolide, romergoline, ropinirole, rotigotine, roxindole and sumanirole, each also in a pharmaceutical composition in dosage unit form comprising, as an active ingredient, said DA-agonist in an amount per unit form of from 0.001 mg to 200 mg, in admixture with a pharmaceutical carrier or vehicle. For this use, said domperidone or pharmaceutically acceptable salt or solvate thereof is formulated in a pharmaceutical composition comprising said domperidone or pharmaceutically acceptable salt or solvate thereof in an amount per unit form (in domperidone base) of from 2 mg to 120 mg, or from 2 mg to 40 mg in admixture with a pharmaceutical carrier, and is administered at a daily dose (in domperidone base) of from 4 mg to 120 mg or from 4 mg to 40 mg.

In particular, according to this second embodiment, in combination with domperidone, said DA-agonist is selected from the group consisting of apomorphine and pharmaceutically acceptable salts and solvates thereof, in an amount per unit dose equivalent to from 1 mg to 2 mg of apomorphine base; bromocriptine and pharmaceutically acceptable salts and solvates thereof, in an amount per unit form equivalent to from 2.5 mg to 200 mg of bromocriptine base, at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; pramipexole, in an amount per unit form equivalent to from 0.125 mg to 45 mg of pramipexole dihydrochloride monohydrate, to be administered at a daily dose (in pramipexole dihydrochloride monohydrate) of from 0.375 mg to 45 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 20 mg of ropinirole hydrochloride, to be administered at a daily dose (in ropinirole hydrochloride) of from 0.25 mg to 20 mg; and rotigotine and pharmaceutically acceptable salts and solvates thereof, in an amount per unit form releasing from 2 mg to 24 mg of rotigotine base, to be administered at a daily dose equivalent to from 2 mg to 24 mg of rotigotine base.

Said pharmaceutical composition comprising said domperidone and said pharmaceutical composition comprising said DA-agonist thus obtained are concurrently or sequentially administered to a patient suffering from a PMND such as PD and PD-related disorders, in particular MSA, DLB, LBD, RLS, DDS, FTLD, PSP, and CBD.

In particular, domperidone, as a pharmaceutical composition comprising domperidone or a pharmaceutically acceptable salt or solvate thereof in an amount per unit form equivalent to from 2 mg to 120 mg, normally from 2 mg to 40 mg, of domperidone base, is concurrently or sequentially administered to said patient at a daily dose equivalent to from 4 mg to 120 mg, normally from 4 mg to 40 mg, of domperidone base, in combination with said pramipexole DA-agonist, also in a pharmaceutical composition comprising pramipexole or a pharmaceutically acceptable salt or solvate thereof in an amount per unit form equivalent to from 0.125 mg to 45 mg of pramipexole dihydrochloride monohydrate, at the aforementioned daily dose.

According to a third embodiment of this second aspect, in the combination of the present invention said AEsI is formulated in a pharmaceutical composition in dosage unit form and is for use in the treatment of a patient suffering from a PD-related disorder selected from the group consisting of DDS, FTLD, PSP and CBD, and said DA-agonist is pramipexole or a pharmaceutically acceptable salt or solvate thereof, also formulated in a pharmaceutical composition in admixture with a pharmaceutical carrier or vehicle.

Thus, the invention also provides an AEsI, in particular selected among the above (A)-(C)AEsIs, in said pharmaceutical composition, in the above dose per unit form, in admixture with a pharmaceutical carrier or vehicle, for use for the treatment of DDS, FTLD, PSP, and CBD, in combination with pramipexole or a pharmaceutically acceptable salt or solvate thereof, in said pharmaceutical composition, at the dose per unit form described in “The DA-agonist Component (b)” section.

Said pharmaceutical (A)-(C)AEsI-composition comprising said AEsI is administered to a patient suffering from DDS, FTLD, PSP, or CBD, in combination with an effective dose of a DA-agonist selected from the group consisting of pramipexole and pharmaceutically acceptable salts and solvates thereof.

In particular, for this use, the AEsI is formulated in a pharmaceutical composition in dosage unit form comprising, as an active ingredient, said AEsI in an amount per unit form of from 1 μg to 600 mg, in admixture with a pharmaceutical carrier or vehicle. This medicament is administered to a patient suffering from DDS, FTLD, PSP, and CBD in combination with a pramipexole or pharmaceutically acceptable salt or solvate thereof at a daily dose equivalent to from 0.375 mg to 45 mg, normally from 1.5 mg to 25 mg or from 1.5 mg to 20 mg, of pramipexole dihydrochloride monohydrate. These daily doses include low pramipexole daily doses useful for the administration during the titration period. At the end of said titration period, the medicament thus manufactured enables the safe intake of pramipexole daily doses never heretofore attained (without the combination with the AEsI).

Preferably, said AEsI is at least one selected from group consisting of ondansetron and pharmaceutically acceptable salt and solvates and prodrugs thereof, in an amount per unit form equivalent to from 2 mg to 32 mg of ondansetron base, and dolasetron and pharmaceutically acceptable salts and solvates and prodrugs thereof, in an amount per unit form equivalent to 25 mg to 200 mg of dolasetron mesylate; aprepitant and pharmaceutically acceptable salt and solvates and prodrugs thereof, in an amount per unit form equivalent to from 10 mg to 250 mg of aprepitant base, and rolapitant and pharmaceutically acceptable salts and solvates and prodrugs thereof, in an amount per unit form equivalent to 15 mg to 270 mg of rolapitant base; and said DA-agonist in said composition is pramipexole or a pharmaceutically acceptable salt or solvate thereof, in an amount per unit form equivalent to from 0.125 mg to 45 mg of pramipexole dihydrochloride monohydrate, to be administered at a daily dose equivalent to from 0.375 mg to 45 mg, normally from 1.5 mg to 45 mg, preferably from more than 4.5 mg to 45 mg, normally from 15 mg to 25 mg or from more than 20 mg to 25 mg of pramipexole dihydrochloride monohydrate.

Advantageously, for this use, said AEsI and said pramipexole are each formulated in a pharmaceutical composition in admixture with a pharmaceutical carrier or vehicle, wherein said AEsI is at least one selected from group consisting of domperidone and pharmaceutically acceptable salt and solvates thereof, in an amount per unit form equivalent to from 2 mg to 120 mg, normally from 2 mg to 40 mg of domperidone base; ondansetron and pharmaceutically acceptable salt and solvates and prodrugs thereof, in an amount per unit form equivalent to from 2 mg to 32 mg of ondansetron base, and dolasetron and pharmaceutically acceptable salts and solvates and prodrugs thereof, in an amount per unit form equivalent to 25 mg to 200 mg of dolasetron mesylate; aprepitant and pharmaceutically acceptable salt and solvates and prodrugs thereof, in an amount per unit form equivalent to from 10 mg to 250 mg of aprepitant base, and rolapitant and pharmaceutically acceptable salts and solvates and prodrugs thereof, in an amount per unit form equivalent to 15 mg to 270 mg of rolapitant base; and said dopamine agonist is pramipexole or a pharmaceutically acceptable salt thereof, in an amount per unit form equivalent to from 0.125 mg to 45 mg, advantageously from 1.5 mg to 45 mg, in particular from 1.5 mg to 25 mg or from 1.5 mg to 20 mg of pramipexole dihydrochloride monohydrate.

The pharmaceutical compositions thus obtained are concurrently or sequentially administered to a patient suffering from MSA, DLB, LBD, DDS, FTLD, PSP, or CBD.

Preferably, the present invention provides a peripheral DA-antagonist selected from the group consisting of domperidone and pharmaceutically acceptable salt and solvates thereof, for use in the treatment of PD in a patient, in combination with a DA-agonist selected from the group consisting of pramipexole and pharmaceutically acceptable salts and solvates thereof, at a daily dose equivalent to from 0.375 mg to 45 mg, from more than 4.5 mg to 45 mg, normally from 15 mg to 25 mg or from more than 20 mg to 25 mg of pramipexole dihydrochloride monohydrate.

Said domperidone DA-antagonist is concurrently or sequentially administered to said patient, in a pharmaceutical composition comprising domperidone or a pharmaceutically acceptable salt or solvate thereof in an amount per unit form equivalent to from 2 mg to 120 mg, normally from 2 mg to 40 mg, of domperidone base, at a daily dose equivalent to from 4 mg to 120 mg, normally from 4 mg to 40 mg, of domperidone base, in combination with said pramipexole DA-agonist, also in a pharmaceutical composition comprising pramipexole or a pharmaceutically acceptable salt or solvate thereof in an amount per unit form equivalent to from 0.125 mg to 45 mg of pramipexole dihydrochloride monohydrate, at the aforementioned daily dose.

Specifically, the invention provides a pharmaceutical composition for use for the treatment of a protein misfolding neurodegenerative disease (PMND) in a patient, which comprises a pharmaceutical carrier or vehicle and a fixed-dose combination of at least one dopamine-agonist Adverse Effects (or Events) Inhibitor (AEsI) selected from the group consisting of domperidone and pharmaceutically acceptable salts and solvates thereof, in an amount equivalent to from 2 mg to 120 mg, of domperidone base, and a dopamine agonist selected from the group consisting of pramipexole and pharmaceutically acceptable salts and solvates thereof, in an amount equivalent to from 0.125 mg to 45 mg of pramipexole dihydrochloride monohydrate. This composition allows a safe, concurrent administration of the domperidone and pramipexole combination to a patient suffering from a PMND such as PD and PD-related disorders.

Pramipexole amounts per unit form and daily doses are described in “The DA-agonist Component (b)” section. Specifically, the pramipexole amount (in pramipexole dihydrochloride monohydrate) per unit form is in a range selected from the group consisting of from 0.125 mg to 45 mg, from 0.375 mg to 45 mg, form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg.

The above amounts per unit form include low amounts to be used during the titration period. During the effective treatment of for example PD, a preferred composition comprises from more than 4.5 mg to 45 mg, normally from more than 4.5 mg to 35 mg, from more than 4.5 mg to 25 mg, from 15 mg to 25 mg or from more than 20 mg to 25 mg This composition may be used for at least arresting or slowing the progression of the disease.

In said combination, including fixed-dose combinations, said pramipexole or pharmaceutically acceptable salt or solvate thereof is normally present, in said pharmaceutical composition, in an amount per unit form equivalent to from 0.125 mg to 10 mg up to from 7.5 mg to 10 mg of pramipexole dihydrochloride monohydrate, in admixture with a pharmaceutical carrier or vehicle for an immediate-release formulation. For the administration of pramipexole at higher doses, said amount per IR-unit form will normally be equivalent to from 1.5 mg to 22.5 mg, from 1.6 mg to 22.5 mg, from more than 4.5 mg to 22.5 mg and from 7.5 mg to 22.5 mg, of pramipexole dihydrochloride monohydrate.

Said pramipexole or pharmaceutically acceptable salt or solvate thereof will normally be present, in said pharmaceutical composition, in an amount per unit form equivalent to 1.5 mg to 45 mg or from more than 4.5 mg to 25 mg, from 15 mg to 25 mg or from more than 20 mg to 25 mg, of pramipexole dihydrochloride monohydrate, in admixture with a pharmaceutical carrier or vehicle for an extended-release formulation. For the administration of pramipexole at higher doses, said amount per ER-unit form will be equivalent to from 15 mg to 45 mg or from 20.25 mg to 25 mg of pramipexole dihydrochloride monohydrate.

According to the above second and third embodiment of this second aspect of the invention, the pramipexole amounts per unit form and daily doses are those described in “The DA-agonist Component (b)” section. Specifically, in the second and third embodiments of this section, the pramipexole amount (in pramipexole dihydrochloride monohydrate) per unit form is in a range selected from the group consisting of from 0.125 mg to 45 mg, from 0.375 mg to 45 mg, form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg. Normally said range is from 15 mg to 25 mg or from more than 20 mg to 25 mg.

The above amounts per unit form include low amounts per unit form to be used during the titration period. During the effective treatment of a PMND such as PD or a PD-related disorder selected from the group consisting of MSA, DLB, LBD, DDS, FTLD, PSP, and CBD, composition comprising more than 4.5 mg to 45 mg, normally from more than 4.5 mg to 35 mg or from more than 4.5 mg to 30 mg, normally from 15 mg to 25 mg, may be used for at least arresting or slowing the progression of the disease.

In particular, for its use for the treatment of MSA, DLB, LBD, RLS, DDS, FTLD, PSP, and CBD, pramipexole or pharmaceutically acceptable salt or solvate thereof is administered to a patient in need of said treatment at a daily dose equivalent to from 0.125 mg to 45 mg, normally from 0.375 mg to 45 mg, from more than 4.5 mg to 45 mg, from 4.8 mg to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg; and from 15 mg to 45 mg, normally from 15 mg to 25 mg or from more than 20 mg to 25 mg of pramipexole dihydrochloride monohydrate.

In said combination, including fixed-dose combinations with said AEsI, in particular with one of the above (A)-(C)AEsIs, in a pharmaceutical composition in dosage unit form, pramipexole or a pharmaceutically acceptable salt thereof, said pramipexole or pharmaceutically acceptable salt or solvate thereof is normally present, in said pharmaceutical composition, in an amount per unit form equivalent to from 0.125 mg to 10 mg up to from 7.5 mg to 10 mg of pramipexole dihydrochloride monohydrate, in admixture with a pharmaceutical carrier or vehicle for an immediate-release formulation. For the administration of pramipexole at higher doses, said amount per IR-unit form will be equivalent to from 1.5 mg to 22.5 mg, from 1.6 mg to 22.5 mg, from more than 4.5 mg to 22.5 or from 7.5 mg to 22.5 mg, normally from 1.5 mg to 20 mg, of pramipexole dihydrochloride monohydrate.

Said pramipexole or pharmaceutically acceptable salt or solvate thereof will be present, in said pharmaceutical composition, in an amount per unit form equivalent to 1.5 mg to 45 mg or from more than 4.5 mg to 20 mg up to from 15 mg to 20 mg, of pramipexole dihydrochloride monohydrate, in admixture with a pharmaceutical carrier or vehicle for an extended-release formulation. For the administration of pramipexole at higher doses, said amount per ER-unit form will be equivalent to from more than 4.5 mg to 45 mg, up to or from 15 mg to 45 mg, normally from 15 mg to 25 mg or from more than 20 mg to 25 mg of pramipexole dihydrochloride monohydrate.

Pramipexole, in a pharmaceutical composition in dosage unit form, is in some cases administered to a patient suffering from MSA, DLB, LBD, DDS, FTLD, PSP, or CBD at a daily dose equivalent to from 0.375 mg to 20-21 mg, from 1.6 mg to 20-21 mg, from 1.625 mg to 20-21 mg, from 3 mg to 20-21 mg, from more than 4.5 mg to 20-21 mg, from 4.8 mg to 20-21 mg, from more than 6 mg to 20-21 mg, from 6.5 mg to 20-21 mg, from 10 mg to 20-21 mg, from 13.5 mg to 20-21 mg, from 14.5 mg to 45 mg; and from 15 mg to 20-21 mg, in particular from 1.5 mg to 20 mg, of pramipexole dihydrochloride monohydrate.

According to a fourth embodiment, in the combination of the present invention said AEsI is formulated in a pharmaceutical composition in dosage unit form and is for use in the treatment of RLS in combination with a DA-agonist selected from the group consisting of pramipexole and pharmaceutically acceptable salts and solvates thereof, also formulated in a pharmaceutical composition in admixture with a pharmaceutical carrier or vehicle.

Accordingly, the invention also provides an AEsI, in particular selected among the above (A)-(B)AEsIs, in said pharmaceutical composition, in the above dose per unit form, in admixture with a pharmaceutical carrier or vehicle, for use for the treatment of RLS, in combination with a DA-agonist selected from the group consisting of pramipexole and pharmaceutically acceptable salts and solvates thereof, also in a pharmaceutical composition comprising said pramipexole or pharmaceutically acceptable salt or solvate thereof in an amount per unit form equivalent to from 0.125 mg to 6 mg of pramipexole dihydrochloride monohydrate.

In particular, said AEsI in said composition is selected from group consisting of domperidone and pharmaceutically acceptable salt and solvates thereof, in an amount per unit form equivalent to from 2 mg to 120 mg, normally from 2 mg to 40 mg of domperidone base, to be administered at a daily dose equivalent to from 4 mg to 120 mg, normally from 4 mg to 40 mg of domperidone base; ondansetron and pharmaceutically acceptable salt and solvates and prodrugs thereof, in an amount per unit form equivalent to from 2 mg to 32 mg of ondansetron base, to be administered at a daily dose equivalent to from 6 mg to 64 mg, normally from 6 mg to 32 mg of ondansetron base; dolasetron and pharmaceutically acceptable salts and solvates and prodrugs thereof, in an amount per unit form equivalent to 25 mg to 200 mg of dolasetron mesylate, to be administered at a daily dose equivalent to from 75 mg to 200 mg of dolasetron mesylate; palonosetron and pharmaceutically acceptable salts and solvates and prodrugs thereof, in an amount per unit form equivalent to from 0.25 mg to 0.5 mg of palonosetron base, to be administered at a daily dose equivalent to from 0.75 to 2 mg of palonosetron base; aprepitant and pharmaceutically acceptable salts and solvates and prodrugs thereof, in an amount per unit form and at a daily dose equivalent to from 10 mg to 250 mg of aprepitant; fosaprepitant and pharmaceutically acceptable salts and solvates and prodrugs thereof, in an amount per unit form and at a daily dose equivalent to from 10 mg to 250 mg of aprepitant; rolapitant and pharmaceutically acceptable salts and solvates and prodrugs thereof, in an amount per unit form and at a daily dose equivalent to from 15 mg to 270 mg of rolapitant; netupitant and pharmaceutically acceptable salts and solvates and prodrugs thereof, in an amount per unit form and at a daily dose equivalent to from 300 mg to 600 mg; netupitant-300/palonosetron-0.5; and fosnetupitant-235/palonosetron-0.25 once a day; and, respectively, said DA-agonist in said composition is selected from the group consisting of pramipexole and pharmaceutically acceptable salts and solvates thereof, in an amount per unit form equivalent to from 0.125 mg to 6 mg, from 0.125 mg to 1 mg, from 0.125 mg to 0.75 mg, and from 0.125 mg to 0.5 mg of pramipexole dihydrochloride monohydrate, to be administered at a daily dose equivalent to from 0.125 mg to 6 mg, from 0.125 mg to 1 mg, from 0.125 mg to 0.75 mg, and from 0.125 mg to 0.5 mg of pramipexole dihydrochloride monohydrate.

According to a specific embodiment, for the use of the AEsI for treating RLS in a patient according to the present invention, the AEsI/DA-agonist combination comprises or consists of an AEsI selected from the group consisting of domperidone base, domperidone maleate and domperidone succinate (1:1), at an effective daily dose equivalent to from 4 mg to 40 mg of domperidone base, in combination with a DA-agonist selected from the group consisting of pramipexole base and pramipexole dihydrochloride monohydrate, at an effective daily dose equivalent to from 0.125 mg to 6 mg, from 0.125 mg to 1 mg, from 0.125 mg to 0.75 mg, or from 0.125 mg to 0.5 mg of pramipexole dihydrochloride monohydrate.

The use according to this second aspect of the present invention is made at the doses per unit form, the maintenance doses of AEsI and DA-agonist and under conditions illustrated herein above for carrying out the method of treatment according to the first aspect of the invention.

### Third Aspect of the Invention

According to a third aspect, the invention provides the use of an AEsI Component (a) for the preparation of a medicament for the treatment of a PMND, in particular of PD and PD-related disorders such as MSA, DLB, LBD, RLS, DDS, FTLD, PSP, and CBD in a patient in need of said treatment, in combination with an effective daily dose of a DA-agonist Component (b). Said effective daily dose of said DA-agonist may be higher, and even much higher than the maximum recommended dose used in the treatment of PD.

The invention also provides the use of an AEsI, in particular one of the above (A)-(C)AEsIs, for the preparation of a medicament including a pharmaceutical composition in dosage unit form comprising, as an active ingredient, said AEsI, in particular one of the above (A)-(C)AEsIs, in a dose per unit form illustrated in “The AEsI Component (a)” section, in admixture with a pharmaceutical carrier or vehicle, for the treatment of a PMND, such as PD and PD-related disorders, in combination with an effective daily dose of a DA-agonist, as illustrated above in “The DA-agonist Component (b)” section.

In particular, the invention provides the use of a of at least one AEsI Component (a) for the preparation of a medicament for the treatment of a PD-related disorder selected from the group consisting of RLS, DDS, FTLD, PSP, and CBD in combination with an effective dose of a DA-agonist selected from the group consisting of pramipexole and pharmaceutically acceptable salts and solvates thereof. Said effective daily dose of said DA-agonist may be higher, and even much higher than the maximum recommended dose used in the treatment of PD.

Said medicament normally is a pharmaceutical composition in dosage unit form comprising, as an active ingredient, at least one of said (A)-(C)AEsI in an amount per unit form of from 1 μg to 600 mg, in admixture with a pharmaceutical carrier or vehicle. This medicament is for the treatment of PD and PD-related disorders, at a daily dose of from 1 μg to 600 mg, in combination with a DA-agonist daily dose of from 0.025 mg to 1000 mg, in a patient in need of said treatment.

Said pharmaceutical composition according to the present invention may comprise at least one AEsI selected from the group consisting of

(A) a 5HT3-antagonist selected from the group consisting of azasetron and pharmaceutically acceptable salts and solvates thereof, in an amount per unit form equivalent to from 5 mg to 10 mg of azasetron hydrochloride, to be administered at a daily dose equivalent to from 15 mg to 40 mg of azasetron hydrochloride; dolasetron and pharmaceutically acceptable salts and solvates thereof, in an amount per unit form equivalent to from 25 mg to 200 mg of dolasetron mesylate, to be administered at a daily dose equivalent to from 75 mg to 200 mg of dolasetron mesylate; granisetron and pharmaceutically acceptable salts and solvates thereof, in an amount per unit form equivalent to from 0.5 mg to 2 mg granisetron base, to be administered at a daily dose equivalent to from 1.5 mg to 8 mg of granisetron base; ondansetron and pharmaceutically acceptable salts and solvates thereof, in an amount per unit form equivalent to from 0.5 mg to 16 mg, normally from 2 mg to 8 mg of ondansetron base, to be administered at a daily dose equivalent to from 6 mg to 64 mg, normally from 6 mg to 32 mg of ondansetron base; palonosetron and pharmaceutically acceptable salts and solvates thereof, in an amount per unit form equivalent to from 0.25 mg to 0.5 mg of palonosetron base, to be administered at a daily dose equivalent to from 0.75 to 2 mg of palonosetron base; ramosetron and pharmaceutically acceptable salts and solvates thereof, in an amount per unit form equivalent to from 0.05 mg to 0.2 mg of ramosetron hydrochloride, to be administered at a daily dose equivalent to from 0.05 mg to 0.2 mg of ramosetron hydrochloride; and tropisetron and pharmaceutically acceptable salts and solvates thereof, in an amount per unit form equivalent to from 2.5 mg to 5 mg of tropisetron base, to be administered at a daily dose equivalent to from 7.5 mg to 20 mg of tropisetron base; and

(B) a NK1-antagonist selected from the group consisting of aprepitant and pharmaceutically acceptable salts and solvates thereof, in an amount per unit form and at a daily dose equivalent to from 10 mg to 250 mg of aprepitant; fosaprepitant and pharmaceutically acceptable salts and solvates thereof, in an amount per unit form and at a daily dose equivalent to from 10 mg to 250 mg of aprepitant; rolapitant and pharmaceutically acceptable salts and solvates thereof, in an amount per unit form and at a daily dose equivalent to from 15 mg to 270 mg of rolapitant, netupitant and pharmaceutically acceptable salts and solvates thereof, in an amount per unit form and at a daily dose equivalent to from 300 mg to 600 mg; netupitant-300/palonosetron-0.5 once a day; and fosnetupitant-235/palonosetron-0.25, administered once a day; and

(C) a peripheral DA-antagonist selected from the group consisting of domperidone and pharmaceutically acceptable salts and solvates thereof, in an amount per unit form equivalent to from 2 mg to 120 mg, normally from 2 mg to 40 mg of domperidone base, to be administered at a daily dose equivalent to from 4 mg to 120 mg, normally from 4 mg to 40 mg of domperidone base.

This composition is administered for the treatment of a disease selected from the group consisting of PD and PD-related disorders in combination with a DA-agonist.

Advantageously, said (A)-(C)AEsI is selected from the group consisting of
domperidone and pharmaceutically acceptable salt and solvates thereof, in an amount per unit form equivalent to from 2 mg to 40 mg of domperidone base, to be administered at a daily dose equivalent to from 4 mg to 40 mg of domperidone base;
ondansetron and pharmaceutically acceptable salt and solvates and prodrugs thereof, in an amount per unit form equivalent to from 2 mg to 32 mg of ondansetron base, to be administered at a daily dose equivalent to from 6 mg to 64 mg, normally from 6 mg to 32 mg of ondansetron base;
dolasetron and pharmaceutically acceptable salts and solvates and prodrugs thereof, in an amount per unit form equivalent to 25 mg to 200 mg of dolasetron mesylate, to be administered at a daily dose equivalent to from 75 mg to 200 mg of dolasetron mesylate;
palonosetron and pharmaceutically acceptable salts and solvates and prodrugs thereof, in an amount per unit form equivalent to from 0.25 mg to 0.5 mg of palonosetron base, to be administered at a daily dose equivalent to from 0.75 to 2 mg of palonosetron base;
aprepitant and pharmaceutically acceptable salts and solvates and prodrugs thereof, in an amount per unit form and at a daily dose equivalent to from 10 mg to 250 mg of aprepitant;
fosaprepitant and pharmaceutically acceptable salts and solvates and prodrugs thereof, in an amount per unit form and at a daily dose equivalent to from 10 mg to 250 mg of aprepitant;
rolapitant and pharmaceutically acceptable salts and solvates and prodrugs thereof, in an amount per unit form and at a daily dose equivalent to from 15 mg to 270 mg of rolapitant,
netupitant and pharmaceutically acceptable salts and solvates and prodrugs thereof, in an amount per unit form and at a daily dose equivalent to from 300 mg to 600 mg;
netupitant-300/palonosetron-0.5;
fosnetupitant-235/palonosetron-0.25; and
domperidone and pharmaceutically acceptable salts and solvates thereof, in an amount per unit form equivalent to from 2 mg to 120 mg, normally from 2 mg to 40 mg of domperidone base and ad at a daily dose equivalent to from 4 mg to 120 mg, normally from 4 mg to 40 mg of domperidone base.

Each of the above AEsI are administered at the respective daily dose for the treatment of a PMND selected from the group consisting of PD and PD-related disorders in combination with a DA-agonist, administered to said patient at a daily dose of from 0.025 mg 1000 mg.

According to a first embodiment of this third aspect of the invention, said pharmaceutical composition comprising at least one of said AEI is administered to a patient suffering from a PMND such as PD or a PD-related disorder, in combination with a DA-agonist selected from the group consisting of apomorphine, bromocriptine, cabergoline, ciladopa dihydrexidine, dihydrexidine, dihydroergocryptine, dinapsoline doxanthrine, lisuride, pergolide, piribedil, propylnorapomorphine, quinagolide, romergoline, ropinirole, rotigotine, roxindole and sumanirole, each also in a pharmaceutical composition in dosage unit form comprising, as an active ingredient, said DA-agonist in an amount per unit form of from 0.001 mg to 200 mg, in admixture with a pharmaceutical carrier or vehicle.

In particular, according to this first embodiment, in combination with the above composition, said DA-agonist, also in a pharmaceutical composition, is selected from the group consisting of apomorphine and pharmaceutically acceptable salts and solvates thereof, in an amount per unit dose equivalent to from 1 mg to 2 mg of apomorphine base, to be administered at a daily dose (in apomorphine base) of from 2 mg to 12 mg; bromocriptine and pharmaceutically acceptable salts and solvates thereof, in an amount per unit form equivalent to from 2.5 mg to 200 mg of bromocriptine base, to be administered at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, to be administered at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, to be administered at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, to be administered at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, to be administered at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 75 mg of ropinirole base, to be administered at a daily dose (in ropinirole base) of from 0.25 mg to 75 mg; and rotigotine and pharmaceutically acceptable salts and solvates thereof, in an amount per unit form releasing from 2 mg to 24 mg of rotigotine base, to be administered at a daily dose equivalent to from 2 mg to 24 mg of rotigotine base; for the treatment of a patient suffering from a PMND selected from the group consisting of PD and PD-related disorders, in particular from a PD-related disorder selected from the group consisting of MSA, DLB, LBD, RLS, DDS, FTLD, PSP, and CBD.

According to a second embodiment, the invention provides the use of at least one of AEsI selected from the group consisting of domperidone and pharmaceutically acceptable salts thereof for the preparation of a medicament for the treatment of a PMND, in combination with a DA-agonist selected from the group consisting of apomorphine, bromocriptine, cabergoline, ciladopa dihydrexidine, dihydrexidine, dihydroergocryptine, dinapsoline doxanthrine, lisuride, pergolide, piribedil, pramipexole, propylnorapomorphine, quinagolide, romergoline, ropinirole, rotigotine, roxindole and sumanirole, each also in a pharmaceutical composition in dosage unit form comprising, as an active ingredient, said DA-agonist in an amount per unit form of from 0.001 mg to 200 mg, in admixture with a pharmaceutical carrier or vehicle.

In particular, according to this second embodiment, in combination with domperidone, said DA-agonist is selected from the group consisting of apomorphine and pharmaceutically acceptable salts and solvates thereof, in an amount per unit dose equivalent to from 1 mg to 2 mg of apomorphine base; bromocriptine and pharmaceutically acceptable salts and solvates thereof, in an amount per unit form equivalent to from 2.5 mg to 200 mg of bromocriptine base, at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, at a daily dose (in pergolide base) of from 0.05 mg to 6 mg; piribedil and pharmaceutically acceptable salts and solvates thereof, in an amount per unit form equivalent to from 20 mg to 200 mg of piribedil base, to be administered at a daily dose (in piribedil base) of from 150 mg to 1000 mg; pramipexole, in an amount per unit form equivalent to from 0.125 mg to 45 mg of pramipexole dihydrochloride monohydrate, to be administered at a daily dose (in pramipexole dihydrochloride monohydrate) of from 0.375 mg to 45 mg; quinagolide and pharmaceutically acceptable salts and solvates thereof, in an amount per unit form equivalent to from 0.025 mg to 0.5 mg of quinagolide base, to be administered at a daily dose (in quinagolide base) of from 0.025 mg to 0.9 mg; ropinirole and pharmaceutically acceptable salts and solvates thereof, in an amount per unit form equivalent to from 0.25 mg to 20 mg of ropinirole hydrochloride, to be administered at a daily dose (in ropinirole hydrochloride) of from 0.25 mg to 20 mg; and rotigotine and pharmaceutically acceptable salts and solvates thereof, in an amount per unit form releasing from 2 mg to 24 mg of rotigotine base, to be administered at a daily dose equivalent to from 2 mg to 24 mg of rotigotine base.

More particularly, according to this second embodiment of this third aspect, the present invention provides the use of a DA-antagonist selected from the group consisting of domperidone and pharmaceutically acceptable salt and solvates thereof, for the preparation of a medicament for the treatment of a PMND, for example AD, PD or a PD-related disorder such as MSA, DLB, LBD, RLS, DDS, FTLD, PSP, and CBD in a patient, in combination with a DA-agonist selected from the group consisting of pramipexole and pharmaceutically acceptable salts and solvates thereof, at a daily dose equivalent to from 0.125 mg to 45 mg, normally from 0.375 mg to 45 mg, from 15 mg to 25 mg or from more than 20 mg to 25 mg, of pramipexole dihydrochloride monohydrate.

Said domperidone DA-antagonist medicament, as a pharmaceutical composition comprising domperidone or a pharmaceutically acceptable salt or solvate thereof in an amount per unit form equivalent to from 2 mg to 120 mg, normally from 2 mg to 40 mg, of domperidone base, is concurrently or sequentially administered to said patient at a daily dose equivalent to from 4 mg to 120 mg, normally from 4 mg to 40 mg, of domperidone base, in combination with said pramipexole DA-agonist, also in a pharmaceutical composition comprising pramipexole or a pharmaceutically acceptable salt or solvate thereof in an amount per unit form equivalent to from 0.125 mg to 45 mg of pramipexole dihydrochloride monohydrate, at the aforementioned daily dose.

Said pharmaceutical composition comprising at least one of said AEsI and said pharmaceutical composition comprising said DA-agonist thus obtained are concurrently or sequentially administered to a patient suffering from a PMND such as PD and PD-related disorders, in particular MSA, DLB, LBD, RLS, DDS, FTLD, PSP, and CBD.

According to a third embodiment of this third aspect of the invention, said pharmaceutical (A)-(C)AEsI-composition comprising at least one of said AEI is administered to a patient suffering from DDS, FTLD, PSP, or CBD, in combination with an effective dose of a DA-agonist selected from the group consisting of pramipexole and pharmaceutically acceptable salts and solvates thereof.

In particular, said medicament is a pharmaceutical composition in dosage unit form comprising, as an active ingredient, said at least one AEsI in an amount per unit form of from 1 μg to 600 mg, in admixture with a pharmaceutical carrier or vehicle. This medicament is administered to a patient suffering from DDS, FTLD, PSP, and CBD in combination with a pramipexole or pharmaceutically acceptable salt or solvate thereof at a daily dose equivalent to from 0.375 mg to 45 mg, normally from 1.5 mg to 25 mg or from 1.5 mg to 20 mg, of pramipexole dihydrochloride monohydrate. These daily doses include low pramipexole daily doses useful for the administration during the titration period. At the end of said titration period, the medicament thus manufactured enables the safe intake of pramipexole daily doses never heretofore attained (without the combination with the AEsI).

According to this third embodiment of this third aspect of the invention said at least one AEsI in said pharmaceutical composition is selected from the group consisting of ondansetron and pharmaceutically acceptable salt and solvates and prodrugs thereof, in an amount per unit form equivalent to from 2 mg to 32 mg of ondansetron base; dolasetron and pharmaceutically acceptable salts and solvates and prodrugs thereof, in an amount per unit form equivalent to 25 mg to 200 mg of dolasetron mesylate; palonosetron and pharmaceutically acceptable salts and solvates and prodrugs thereof, in an amount per unit form equivalent to from 0.25 mg to 0.5 mg of palonosetron base; aprepitant and pharmaceutically acceptable salts and solvates and prodrugs thereof, in an amount per unit form and at a daily dose equivalent to from 10 mg to 250 mg of aprepitant base; fosaprepitant and pharmaceutically acceptable salts and solvates and prodrugs thereof, in an amount per unit form equivalent to from 10 mg to 250 mg of aprepitant base; rolapitant and pharmaceutically acceptable salts and solvates and prodrugs thereof, in an amount per unit form equivalent to from 15 mg to 270 mg of rolapitant base; netupitant and pharmaceutically acceptable salts and solvates and prodrugs thereof, in an amount per unit form equivalent to from 300 mg to 600 mg of netupitant base; netupitant-300/palonosetron-0.5; and fosnetupitant-235/palonosetron-0.25.

In combination with said advantageous AEsIs in said pharmaceutical composition, pramipexole or pharmaceutically acceptable salt thereof is safely administered to a patient suffering from DDS, FTLD, PSP, and CBD at a daily dose as illustrated in “The DA-agonist Component (b)” section, for example at a dose-range equivalent to from 0.375 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg, from 15 mg to 45 mg, from 15 mg to 25 mg or from more than 20 mg to 25 mg, normally in the range of from 1.5 mg to 25 mg or from 1.5 mg to 20 mg of pramipexole dihydrochloride monohydrate.

For their administration for the treatment of DDS, FTLD, PSP, and CBD, the AEsI and pramipexole or pharmaceutically acceptable salt or sulfate thereof are each formulated in a pharmaceutical composition in admixture with a pharmaceutical carrier or vehicle.

These pharmaceutical compositions, constantly used in combination each other, allow for the first time the use of pramipexole for the substantial and efficacious treatment of a patient suffering from DDS, FTLD, PSP, and CBD.

Advantageously,

said at least one AEsI in said composition is selected from group consisting of ondansetron and pharmaceutically acceptable salt and solvates and prodrugs thereof, in an amount per unit form equivalent to from 2 mg to 32 mg of ondansetron base, and dolasetron and pharmaceutically acceptable salts and solvates and prodrugs thereof, in an amount per unit form equivalent to 25 mg to 200 mg of dolasetron mesylate; aprepitant and pharmaceutically acceptable salt and solvates and prodrugs thereof, in an amount per unit form equivalent to from 10 mg to 250 mg of aprepitant base, and rolapitant and pharmaceutically acceptable salts and solvates and prodrugs thereof, in an amount per unit form equivalent to 15 mg to 270 mg of rolapitant base; and

said pramipexole or a pharmaceutically acceptable salt thereof, in an amount per unit form (in pramipexole dihydrochloride monohydrate) of from 0.125 mg to 45 mg, from 0.375 mg to 45 mg, from more than 4.5 mg to 45 mg or from 15 mg to 45 mg, normally from 15 mg to 25 mg or from more than 20 mg to 25 mg.

According to the above second and third embodiment of this second aspect of the invention, the pramipexole amounts per unit form and daily doses are those described in “The DA-agonist Component (b)” section. Specifically, in the second and third embodiments of this section, the pramipexole amount (in pramipexole dihydrochloride monohydrate) per unit form is in a range selected from the group consisting of from 0.125 mg to 45 mg, from 0.375 mg to 45 mg, form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg. Normally said range is from 15 mg to 25 mg or from more than 20 mg to 25 mg.

The above amounts per unit form include low amounts per unit form to be used during the titration period. During the effective treatment of a PMND such as PD or a PD-related disorder selected from the group consisting of MSA, DLB, LBD, DDS, FTLD, PSP, and CBD, composition comprising more than 4.5 mg to 45 mg, normally from more than 4.5 mg to 35 mg or from more than 4.5 mg to 30 mg, normally from 15 mg to 25 mg, may be used for at least arresting or slowing the progression of the disease.

In particular, for its use for the treatment of MSA, DLB, LBD, RLS, DDS, FTLD, PSP, and CBD, pramipexole or pharmaceutically acceptable salt or solvate thereof is administered to a patient in need of said treatment at a daily dose equivalent to from 0.125 mg to 45 mg, normally from 0.375 mg to 45 mg, from more than 4.5 mg to 45 mg, from 4.8 mg to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg; and from 15 mg to 45 mg, normally from 15 mg to 25 mg or from more than 20 mg to 25 mg of pramipexole dihydrochloride monohydrate.

In said combination, including fixed-dose combinations with said AEsI, in particular with at least one of the above (A)-(C)AEsIs, in a pharmaceutical composition in dosage unit form, pramipexole or a pharmaceutically acceptable salt thereof, said pramipexole or pharmaceutically acceptable salt or solvate thereof is normally present, in said pharmaceutical composition, in an amount per unit form equivalent to from 0.125 mg to 10 mg up to from 7.5 mg to 10 mg of pramipexole dihydrochloride monohydrate, in admixture with a pharmaceutical carrier or vehicle for an immediate-release formulation. For the administration of pramipexole at higher doses, said amount per IR-unit form will be equivalent to from 1.5 mg to 22.5 mg, from 1.6 mg to 22.5 mg, from more than 4.5 mg to 22.5 or from 7.5 mg to 22.5 mg, normally from 1.5 mg to 20 mg, of pramipexole dihydrochloride monohydrate.

Said pramipexole or pharmaceutically acceptable salt or solvate thereof will be present, in said pharmaceutical composition, in an amount per unit form equivalent to 1.5 mg to 45 mg or from more than 4.5 mg to 20 mg up to from 15 mg to 20 mg, of pramipexole dihydrochloride monohydrate, in admixture with a pharmaceutical carrier or vehicle for an extended-release formulation. For the administration of pramipexole at higher doses, said amount per ER-unit form will be equivalent to from more than 4.5 mg to 45 mg, up to or from 15 mg to 45 mg, normally from 15 mg to 25 mg or from more than 20 mg to 25 mg of pramipexole dihydrochloride monohydrate.

Pramipexole, in a pharmaceutical composition in dosage unit form, is in some cases administered to a patient suffering from MSA, DLB, LBD, DDS, FTLD, PSP, or CBD at a daily dose equivalent to from 0.375 mg to 20-21 mg, from 1.6 mg to 20-21 mg, from 1.625 mg to 20-21 mg, from 3 mg to 20-21 mg, from more than 4.5 mg to 20-21 mg, from 4.8 mg to 20-21 mg, from more than 6 mg to 20-21 mg, from 6.5 mg to 20-21 mg, from 10 mg to 20-21 mg, from 13.5 mg to 20-21 mg, from 14.5 mg to 45 mg; and from 15 mg to 20-21 mg, in particular from 1.5 mg to 20 mg, of pramipexole dihydrochloride monohydrate.

According to a fourth embodiment of this third aspect of the invention, a pharmaceutical composition comprising at least one AEI selected from the group consisting of 5HT3-antagonists and NK1-antagonists is concurrently or sequentially administered in combination with a DA-agonist selected from the group consisting of pramipexole and pharmaceutically acceptable salts and solvates thereof, also in a pharmaceutical composition administered at an effective daily dose for the treatment of a patient suffering from RLS.

In particular, said pharmaceutical composition is formulated in dosage unit form and is administered to a patient suffering from RLS in combination with a DA-agonist selected from the group consisting of pramipexole and pharmaceutically acceptable salts and solvates thereof, also formulated in a pharmaceutical composition in admixture with a pharmaceutical carrier or vehicle.

Accordingly, the invention also provides the use of at least one of AEsI selected from the group consisting of 5HT3-antagonists and NK1-antagonists, for the preparation of a medicament for the treatment of RLS comprising or consisting of a pharmaceutical composition comprising said AEsI selected from the group consisting of 5HT3-antagonists and NK1-antagonists, in per unit form of from 1 μg to 600 mg, in admixture with a pharmaceutical carrier or vehicle, in combination with a DA-agonist selected from the group consisting of pramipexole and pharmaceutically acceptable salts and solvates thereof, also in a pharmaceutical composition comprising said pramipexole or pharmaceutically acceptable salt or solvate thereof in an amount per unit form equivalent to from 0.125 mg to 6 mg or from 0.125 mg to 1 mg, normally from 0.125 mg to 0.75 mg or from 0.125 mg to 0.50 mg of pramipexole dihydrochloride monohydrate and administered to said patient at a daily dose equivalent to form from 0.125 mg to 6 mg or from 0.125 mg to 1 mg, normally from 0.125 mg to 0.75 mg or from 0.125 mg to 0.50 mg of pramipexole dihydrochloride monohydrate.

In particular, said at least one of AEsI in said composition is selected from group consisting ondansetron and pharmaceutically acceptable salt and solvates and prodrugs thereof, in an amount per unit form equivalent to from 2 mg to 32 mg, normally from 2 mg to 8 mg, of ondansetron base, to be administered at a daily dose equivalent to from 6 mg to 64 mg, normally from 6 mg to 16 mg of ondansetron base; dolasetron and pharmaceutically acceptable salts and solvates and prodrugs thereof, in an amount per unit form equivalent to 25 mg to 200 mg of dolasetron mesylate, to be administered at a daily dose equivalent to from 75 mg to 200 mg of dolasetron mesylate; palonosetron and pharmaceutically acceptable salts and solvates and prodrugs thereof, in an amount per unit form equivalent to from 0.25 mg to 0.5 mg of palonosetron base, to be administered at a daily dose equivalent to from 0.75 to 2 mg of palonosetron base; aprepitant and pharmaceutically acceptable salts and solvates and prodrugs thereof, in an amount per unit form and at a daily dose equivalent to from 10 mg to 250 mg of aprepitant; fosaprepitant and pharmaceutically acceptable salts and solvates and prodrugs thereof, in an amount per unit form and at a daily dose equivalent to from 10 mg to 250 mg of aprepitant; rolapitant and pharmaceutically acceptable salts and solvates and prodrugs thereof, in an amount per unit form and at a daily dose equivalent to from 15 mg to 270 mg of rolapitant; netupitant and pharmaceutically acceptable salts and solvates and prodrugs thereof, in an amount per unit form and at a daily dose equivalent to from 300 mg to 600 mg; netupitant-300/palonosetron-0.5; and fosnetupitant-235/palonosetron-0.25 once a day; and, respectively,

said DA-agonist in said composition is selected from the group consisting of pramipexole and pharmaceutically acceptable salts and solvates thereof, in an amount per unit form equivalent to from 0.125 mg to 6 mg, from 0.125 mg to 1 mg, from 0.125 mg to 0.75 mg, and from 0.125 mg to 0.5 mg of pramipexole dihydrochloride monohydrate, to be administered at a daily dose equivalent to from 0.125 mg to 6 mg, from 0.125 mg to 1 mg, from 0.125 mg to 0.75 mg, and from 0.125 mg to 0.5 mg of pramipexole dihydrochloride monohydrate.

According to this third aspect of the present invention, if the AEsI is ondansetron or pharmaceutically acceptable salt or solvate thereof, in particular ondansetron hydrochloride dihydrate, the dose per tablet to be administered to a patient suffering from a PMND such as DDS, FTLD, PSP, RLS and CBD in combination with pramipexole or pharmaceutically acceptable salt or solvate thereof, will be in a range equivalent to from 2 mg to 32 mg or from 2 mg to 16 mg, normally from 2 mg to 8 mg or from 4 mg to 8 mg of ondansetron base.

Ondansetron may also be present in a composition for transdermal administration, subcutaneous administration, intravenous administration, in a slow-release composition, such as extended release tablets or capsules, or a combination product, for example as a Transdermal Drug Delivery System (TDDS) such as a patch, preferably a matrix patch like that described by Cho J-R et al 2016; a patch pump, an infusion pump, or a micropump; or a fast-dissolving buccal film such as that described by Koland M et al. 2013.

According to this third embodiment, the present invention provides the use of a dopamine-agonist Adverse Effects (or Events) Inhibitor (AEsI) selected from the group consisting of domperidone and pharmaceutically acceptable salts and solvates thereof for the preparation of a medicament for the treatment of a protein misfolding neurodegenerative disease (PMND) in a fixed-dose combination with pramipexole or a pharmaceutically acceptable salt thereof.

In particular, said domperidone or pharmaceutically acceptable salt or solvate thereof, in an amount equivalent to from 2 mg to 120 mg of domperidone base, in a fixed dose combination with said pramipexole or pharmaceutically acceptable salt or and solvate thereof, in an amount equivalent to from 0.125 mg to 45 mg of pramipexole dihydrochloride monohydrate.

More particularly, if the AEsI is domperidone or pharmaceutically acceptable salt or solvate thereof selected from the group consisting of domperidone base, domperidone maleate or domperidone succinate (1:1), the dose per tablet to be administered to a patient suffering from a PMND, for example AD, PD or a PD-related disorder such as MSA, DLB, LBD, RLS, DDS, FTLD, PSP, and CBD in a patient, in combination with pramipexole or pharmaceutically acceptable salt or solvate thereof, will be in a range equivalent to from 2 mg to 120 mg or from 2 mg to 40 mg, from 4 mg to 20 mg or from 4 mg to 10 mg of domperidone base, and is administered to a patient in need of said treatment at a daily dose (in domperidone base) of from 4 mg to 120 mg, normally from 4 mg to 40 mg.

Preferably, according to this third aspect the present invention provides the use of a DA-antagonist selected from the group consisting of domperidone and pharmaceutically acceptable salt and solvates thereof, for the preparation of a medicament for the treatment of PD in a patient, in combination with a DA-agonist selected from the group consisting of pramipexole and pharmaceutically acceptable salts and solvates thereof, at a daily dose equivalent to from 0.125 mg to 45 mg, normally from 0.375 mg to 45 mg, of pramipexole dihydrochloride monohydrate.

Said domperidone DA-antagonist medicament, as a pharmaceutical composition comprising domperidone or a pharmaceutically acceptable salt or solvate thereof in an amount per unit form equivalent to from 2 mg to 120 mg, normally from 2 mg to 40 mg, of domperidone base, is concurrently or sequentially administered to said patient at a daily dose equivalent to from 4 mg to 120 mg, normally from 4 mg to 40 mg, of domperidone base, in combination with said pramipexole DA-agonist, also in a pharmaceutical composition comprising pramipexole or a pharmaceutically acceptable salt or solvate thereof in an amount per unit form equivalent to from 0.125 mg to 45 mg of pramipexole dihydrochloride monohydrate, at the aforementioned daily dose.

Alternatively, the invention provides the use of a dopamine-agonist Adverse Effects (or Events) Inhibitor (AEsI) selected from the group consisting of domperidone and pharmaceutically acceptable salts and solvates thereof for the preparation of a medicament comprising or consisting of a pharmaceutical composition for the treatment of a protein misfolding neurodegenerative disease (PMND) in a patient, which comprises said domperidone or pharmaceutically acceptable salt or solvate thereof, in an amount equivalent to from 2 mg to 120 mg, normally from 2 mg to 40 mg, in a fixed-dose combination with a dopamine agonist selected from the group consisting of pramipexole and pharmaceutically acceptable salts and solvates thereof, in an amount equivalent to from 0.125 mg to 45 mg of pramipexole dihydrochloride monohydrate. This composition allows a safe, concurrent administration of the domperidone and pramipexole combination to a patient suffering from said PMND.

Pramipexole amounts per unit form and daily doses are described in “The DA-agonist Component (b)” section. Specifically, the pramipexole amount (in pramipexole dihydrochloride monohydrate) per unit form is from the group consisting of from 0.125 mg to 45 mg, from 0.375 mg to 45 mg, from more than 4.5 mg to 45 mg, up to from 15 mg to 45 mg, normally from 15 mg to 45 mg or from more than 20 mg to 45 mg.

The above amounts per unit form include low amounts to be used during the titration period. During the effective treatment of PD, composition comprising more than 4.5 mg to 45 mg, normally from more than 4.5 mg to 35 mg or from more than 4.5 mg to 30 mg, normally from 7.5 mg to 25 mg, from 15 mg to 25 mg or from more than 20 mg to 25 mg, may be used for at least arresting or slowing the progression of the disease.

According to the above second and third embodiment of this second aspect of the invention, the pramipexole amounts per unit form and daily doses are those described in “The DA-agonist Component (b)” section. Specifically, in the second and third embodiments of this section, the pramipexole amount (in pramipexole dihydrochloride monohydrate) per unit form is in a range selected from the group consisting of from 0.125 mg to 45 mg, from 0.375 mg to 45 mg, form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg. Normally said range is from 15 mg to 25 mg or from more than 20 mg to 25 mg.

The above amounts per unit form include low amounts per unit form to be used during the titration period. During the effective treatment of a PMND such as PD or a PD-related disorder selected from the group consisting of MSA, DLB, LBD, DDS, FTLD, PSP, and CBD, composition comprising more than 4.5 mg to 45 mg, normally from more than 4.5 mg to 35 mg or from more than 4.5 mg to 30 mg, normally from 15 mg to 25 mg, may be used for at least arresting or slowing the progression of the disease.

In particular, for its use for the treatment of MSA, DLB, LBD, RLS, DDS, FTLD, PSP, and CBD, pramipexole or pharmaceutically acceptable salt or solvate thereof is administered to a patient in need of said treatment at a daily dose equivalent to from 0.125 mg to 45 mg, normally from 0.375 mg to 45 mg, from more than 4.5 mg to 45 mg, from 4.8 mg to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg; and from 15 mg to 45 mg, normally from 15 mg to 25 mg or from more than 20 mg to 25 mg of pramipexole dihydrochloride monohydrate.

In said combination, including fixed-dose combinations with said AEsI, in particular with one of the above (A)-(C)AEsIs, in a pharmaceutical composition in dosage unit form, pramipexole or a pharmaceutically acceptable salt thereof, said pramipexole or pharmaceutically acceptable salt or solvate thereof is normally present, in said pharmaceutical composition, in an amount per unit form equivalent to from 0.125 mg to 10 mg up to from 7.5 mg to 10 mg of pramipexole dihydrochloride monohydrate, in admixture with a pharmaceutical carrier or vehicle for an immediate-release formulation. For the administration of pramipexole at higher doses, said amount per IR-unit form will be equivalent to from 1.5 mg to 22.5 mg, from 1.6 mg to 22.5 mg, from more than 4.5 mg to 22.5 or from 7.5 mg to 22.5 mg, normally from 1.5 mg to 20 mg, of pramipexole dihydrochloride monohydrate.

Said pramipexole or pharmaceutically acceptable salt or solvate thereof will be present, in said pharmaceutical composition, in an amount per unit form equivalent to 1.5 mg to 45 mg or from more than 4.5 mg to 20 mg up to from 15 mg to 20 mg, of pramipexole dihydrochloride monohydrate, in admixture with a pharmaceutical carrier or vehicle for an extended-release formulation. For the administration of pramipexole at higher doses, said amount per ER-unit form will be equivalent to from more than 4.5 mg to 45 mg, up to or from 15 mg to 45 mg, normally from 15 mg to 25 mg or from more than 20 mg to 25 mg of pramipexole dihydrochloride monohydrate.

Pramipexole, in a pharmaceutical composition in dosage unit form, is in some cases administered to a patient suffering from MSA, DLB, LBD, DDS, FTLD, PSP, or CBD at a daily dose equivalent to from 0.375 mg to 20-21 mg, from 1.6 mg to 20-21 mg, from 1.625 mg to 20-21 mg, from 3 mg to 20-21 mg, from more than 4.5 mg to 20-21 mg, from 4.8 mg to 20-21 mg, from more than 6 mg to 20-21 mg, from 6.5 mg to 20-21 mg, from 10 mg to 20-21 mg, from 13.5 mg to 20-21 mg, from 14.5 mg to 45 mg; and from 15 mg to 20-21 mg, in particular from 1.5 mg to 20 mg, of pramipexole dihydrochloride monohydrate.

According to a specific embodiment, in the method (or use) for treating RLS in a patient according to the present invention, the AEsI/DA-agonist combination comprises or consists of an AEsI selected from the group consisting of domperidone base, domperidone maleate and domperidone succinate (1:1), at an effective daily dose equivalent to from 4 mg to 40 mg of domperidone base, in combination with a DA-agonist selected from the group consisting of pramipexole base and pramipexole dihydrochloride monohydrate, at an effective daily dose equivalent to from 0.125 mg to 6 mg, from 0.125 mg to 1 mg, from 0.125 mg to 0.75 mg, or from 0.125 mg to 0.5 mg of pramipexole dihydrochloride monohydrate.

The AEsI Component (a) and the DA-agonist Component (b) can be administered separately or together in any conventional oral or parenteral dosage unit form such as capsule, tablet, powder, cachet, suspension, solution, or transdermal device for the treatment of a PMND such as PD and PD-related disorders, in particular a PD-related disorder selected from the group consisting of RLS, MSA, DDS, FTLD, PSP, and CBD.

In the case of separate (concurrent or sequential) administration of said AEsI, in an effective amount per unit form, and of said DA-agonist, in an effective amount per unit form, each of them can be packaged in a kit comprising said AEsI, in admixture with a pharmaceutical carrier or vehicle, in a container; and said DA-agonist, preferably pramipexole, in admixture with a pharmaceutical carrier or vehicle, in another, separate container.

For their concurrent administration for the treatment of PD and PD-related disorders, said AEsI and said DA-agonist may also be formulated together in a fixed-dose combination consisting of a pharmaceutical composition comprising said AEsI and said DA-agonist, in admixture with a pharmaceutical carrier or vehicle.

In a preferred embodiment, said medicament for the treatment of PD and PD-related disorders according to this section is a pharmaceutical composition in dosage unit form comprising said AEsI is at least one of selected from the group consisting of peripheral DA-antagonists (in particular domperidone), 5HT3-antagonists and NK1-antagonists, in a fixed-dose combination with a DA-agonist, selected from the group consisting of pramipexole and pharmaceutically acceptable salt or solvate thereof, in admixture with a pharmaceutical carrier or vehicle, and said related disorder is RLS. Normally, said composition for the treatment of RLS comprises said AEsI in an amount per unit form of from 1 μg to 600 mg and said DA-agonist in an amount per unit form equivalent to from 0.125 mg to 6 mg of pramipexole dihydrochloride monohydrate.

The fixed-dose combinations assure the safe, concurrent administration of the AEsI and of the DA-agonist.

### Fourth Aspect of the Invention

Thus, according to a fourth aspect, the invention provides a pharmaceutical fixed-dose combination including a pharmaceutical composition comprising said AEsI, in particular at least one of the (A)-(C)AEsIs illustrated above, in an effective dose per unit form, as Component (a) and a DA-agonist, in an effective dose per unit form, as Component (b) in admixture with a pharmaceutical carrier or vehicle. Said fixed-dose combination is for use in the treatment of a PMND such as PD and PD-related disorders.

A fixed-dose combination assures the safe, concurrent administration of the AEsI and of the DA-agonist

The dose per unit form of the AEsI Component (a) ranges from 1 μg to 600 mg; and the dose per unit form of the DA-agonist Component (b) ranges from 0.001 mg to 200 mg.

Said Component (a) may be any of the (A)-(C)AEsIs illustrated in “The AEsI Component (a)” section and in the disclosure of the first aspect of the invention, in the doses per unit form illustrated in said section and disclosure; and said Component (b) may be any DA-agonist illustrated in “The DA-agonist Component (b)” section and in the disclosure of the first aspect of the invention, in the doses per unit form illustrated in said section and disclosure.

According to a first embodiment, said DA-agonist Component (b) is selected from the group consisting of apomorphine, bromocriptine, cabergoline, ciladopa, dihydrexidine, dihydroergocryptine, dinapsoline doxanthrine, epicriptine lisuride, pergolide, piribedil, propylnorapomorphine, quinagolide, romergoline, ropinirole, rotigotine, roxindole, sumanirole and pharmaceutically acceptable salts and solvates and prodrugs of each of these DA-agonists.

In particular, according to this first embodiment, the invention provides a pharmaceutical composition in dosage unit form which comprises
(a) at least one AEsI selected from the group consisting of ondansetron and pharmaceutically acceptable salt and solvates and prodrugs thereof, in an amount per unit form equivalent to from 2 mg to 32 mg of ondansetron base; dolasetron and pharmaceutically acceptable salts and solvates and prodrugs thereof, in an amount per unit form equivalent to 25 mg to 200 mg of dolasetron mesylate; palonosetron and pharmaceutically acceptable salts and solvates and prodrugs thereof, in an amount per unit form equivalent to from 0.25 mg to 0.5 mg of palonosetron base; aprepitant and pharmaceutically acceptable salts and solvates and prodrugs thereof, in an amount per unit form and at a daily dose equivalent to from 10 mg to 250 mg of aprepitant base; fosaprepitant and pharmaceutically acceptable salts and solvates and prodrugs thereof, in an amount per unit form equivalent to from 10 mg to 250 mg of aprepitant base; rolapitant and pharmaceutically acceptable salts and solvates and prodrugs thereof, in an amount per unit form equivalent to from 15 mg to 270 mg of rolapitant base; netupitant and pharmaceutically acceptable salts and solvates and prodrugs thereof, in an amount per unit form equivalent to from 300 mg to 600 mg of netupitant base; and
(b) a DA-agonist selected from the group consisting of apomorphine and pharmaceutically acceptable salts and solvates thereof, in an amount per unit dose equivalent to from 1 mg to 2 mg of apomorphine base; bromocriptine and pharmaceutically acceptable salts and solvates thereof, in an amount per unit form equivalent to from 2.5 mg to 200 mg of bromocriptine base, at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, at a daily dose (in pergolide base) of from 0.05 mg to 6 mg; piribedil and pharmaceutically acceptable salts and solvates thereof, in an amount per unit form equivalent to from 20 mg to 200 mg of piribedil base; quinagolide and pharmaceutically acceptable salts and solvates thereof, in an amount per unit form equivalent to from 0.025 mg to 0.5 mg of quinagolide base; ropinirole and pharmaceutically acceptable salts and solvates thereof, in an amount per unit form equivalent to from 0.25 mg to 75 mg of ropinirole base; and rotigotine and pharmaceutically acceptable salts and solvates thereof, in an amount per unit form (in rotigotine base) releasing from 2 mg to 24 mg of rotigotine base,
   in admixture with a pharmaceutical carrier or vehicle. This composition is for use for the treatment of a PMND such as PD and PD-related disorders; especially MSA, DLB, LBD, RLS, DDS, FTLD, PSP, and CBD, in a patient in need of said treatment.

According to a second embodiment of this fourth aspect, the invention provides a pharmaceutical fixed-dose combination including a pharmaceutical composition comprising
(a) an AEsI selected from the group consisting of domperidone and pharmaceutically acceptable salt and solvates thereof, in an amount per unit form equivalent to from 2 mg to 120 mg, normally from 2 mg to 40 mg of domperidone base; and
(b) a DA-agonist selected from the group consisting of apomorphine and pharmaceutically acceptable salts and solvates thereof, in an amount per unit dose equivalent to from 1 mg to 2 mg of apomorphine base; bromocriptine and pharmaceutically acceptable salts and solvates thereof, in an amount per unit form equivalent to from 2.5 mg to 200 mg of bromocriptine base, at a daily dose (in bromocriptine base) of from 2.5 mg to 200 mg; cabergoline and pharmaceutically acceptable salts and solvates thereof, in an amount per unit form equivalent to from 0.5 mg to 1 mg of cabergoline base, at a weekly dose (in cabergoline base) of from 0.5 mg to 4 mg; dihydroergocryptine and pharmaceutically acceptable salts and solvates thereof, in an amount per unit form equivalent to from 0.3 mg to 40 mg of dihydroergocryptine base, at a daily dose (in dihydroergocryptine base) of from 0.3 mg to 80 mg; lisuride and pharmaceutically acceptable salts and solvates thereof, in an amount per unit form equivalent to from 0.5 mg to 10 mg of lisuride base, at a daily dose (in lisuride base) of from 0.5 mg to 10 mg; pergolide and pharmaceutically acceptable salts and solvates thereof, in particular its mesylate, in an amount per unit form equivalent to from 0.05 mg to 2 mg of pergolide base, at a daily dose (in pergolide base) of from 0.05 mg to 6 mg; piribedil and pharmaceutically acceptable salts and solvates thereof, in an amount per unit form equivalent to from 20 mg to 200 mg of piribedil base; pramipexole, in an amount per unit form equivalent to from 0.125 mg to 45 mg of pramipexole dihydrochloride monohydrate; quinagolide and pharmaceutically acceptable salts and solvates thereof, in an amount per unit form equivalent to from 0.025 mg to 0.5 mg of quinagolide base; ropinirole and pharmaceutically acceptable salts and solvates thereof, in an amount per unit form equivalent to from 0.25 mg to 20 mg of ropinirole hydrochloride; and rotigotine and pharmaceutically acceptable salts and solvates thereof, in an amount per unit form (in rotigotine base) releasing from 2 mg to 24 mg of rotigotine base,
   in admixture with a pharmaceutical carrier or vehicle.

In particular, according to this second embodiment of this fourth aspect, the invention provides a pharmaceutical composition in dosage unit form which comprises
(a) a peripheral DA-antagonist selected from the group consisting of domperidone and pharmaceutically acceptable salt and solvates thereof, in an amount per unit form equivalent to from 2 mg to 120 mg, normally from 2 mg to 40 mg of domperidone base; and
(b) a DA-agonist selected from the group consisting of pramipexole and pharmaceutically acceptable salt and solvates thereof, in an amount per unit form equivalent to from 0.125 mg to 45 mg of pramipexole dihydrochloride monohydrate,
   in admixture with a pharmaceutical carrier or vehicle. This composition is useful or for use for the treatment of a PMND. Said PMND may be AD, PD or a PD-related selected from the group consisting of MSA, DLB, LBD, RLS, DDS, FTLD, PSP, and CBD in a patient.

More particularly, according to this second embodiment, the invention provides a pharmaceutical composition for use for the treatment of a PMND in a patient, comprising a pharmaceutical carrier or vehicle and a fixed-dose combination of domperidone or a pharmaceutically acceptable salt or solvate thereof, in an amount equivalent to from 2 mg to 120 mg of domperidone base, and of pramipexole or a pharmaceutically acceptable salt or solvate thereof, in an amount equivalent to from 0.125 mg to 45 mg of pramipexole dihydrochloride monohydrate.

An advantageous domperidone Component (a) is selected from the group consisting of domperidone base, domperidone maleate and domperidone succinate (1:1).

Preferably, said pharmaceutical compositions comprises said domperidone or pharmaceutically acceptable salt or solvate thereof Component (a) in an amount per unit form equivalent to from 2 mg to 120 mg, normally from 2 mg to 40 mg of domperidone base and pramipexole or a pharmaceutically acceptable salt or solvate thereof, in an amount equivalent to from 0.125 mg to 45 mg, from more than 4.5 mg to 45 mg, up to from 15 mg to 45 mg, normally from more than 4.5 mg to 25 mg, from 15 mg to 25 mg or from more than 20 mg to 25 mg of pramipexole dihydrochloride monohydrate.

Said composition comprising domperidone and pramipexole is to be administered once to three times per day, normally once or twice a day, at a domperidone daily dose equivalent to from 4 mg to 120 mg, normally from 4 mg to 40 mg of domperidone base and at a pramipexole daily dose equivalent to from 4 mg to 120 mg, normally from 4 mg to 40 mg of pramipexole dihydrochloride monohydrate, and to a patient suffering from PD or a PD-related disorder, in combination with a DA-agonist.

According to a third embodiment of this fourth aspect, the invention provides a pharmaceutical fixed-dose combination including a pharmaceutical composition comprising said AEsI, in particular at least one of the (A)-(C)AEsIs illustrated in “The AEsI Component (a)” section above, in an effective dose per unit form, as Component (a) and a DA-agonist selected from the group consisting of pramipexole and pharmaceutically acceptable salts thereof, in an effective dose per unit form, as Component (b) in admixture with a pharmaceutical carrier or vehicle, for use in the treatment of a PD-related disorder selected from the group consisting of DDS, FTLD, PSP, and CBD.

In particular, according to this third embodiment of this fourth aspect, the invention provides a pharmaceutical composition comprising
(a) at least one of AEsI selected from the group consisting of ondansetron and pharmaceutically acceptable salt and solvates and prodrugs thereof, in an amount per unit form equivalent to from 2 mg to 32 mg of ondansetron base; dolasetron and pharmaceutically acceptable salts and solvates and prodrugs thereof, in an amount per unit form equivalent to 25 mg to 200 mg of dolasetron mesylate; palonosetron and pharmaceutically acceptable salts and solvates and prodrugs thereof, in an amount per unit form equivalent to from 0.25 mg to 0.5 mg of palonosetron base; aprepitant and pharmaceutically acceptable salts and solvates and prodrugs thereof, in an amount per unit form and at a daily dose equivalent to from 10 mg to 250 mg of aprepitant base; fosaprepitant and pharmaceutically acceptable salts and solvates and prodrugs thereof, in an amount per unit form equivalent to from 10 mg to 250 mg of aprepitant base; rolapitant and pharmaceutically acceptable salts and solvates and prodrugs thereof, in an amount per unit form equivalent to from 15 mg to 270 mg of rolapitant base; netupitant and pharmaceutically acceptable salts and solvates and prodrugs thereof, in an amount per unit form equivalent to from 300 mg to 600 mg of netupitant base; and
(b) a DA-agonist selected from the group consisting of pramipexole and pharmaceutically acceptable salts and solvates thereof, in an amount per unit form equivalent to from 0.125 mg to 45 mg, preferably from 1.5 mg to 25 mg or from more than 20 mg to 25 mg, normally from 1.5 mg to 20 mg, of pramipexole dihydrochloride monohydrate,
   in admixture with a pharmaceutical carrier or vehicle. This composition is for use in the treatment of DDS, FTLD, PSP, and CBD in a patient in need of said treatment.

Advantageously, in this composition, said at least one AEsI is selected from group consisting of ondansetron and pharmaceutically acceptable salt and solvates and prodrugs thereof, in an amount per unit form equivalent to from 2 mg to 32 mg of ondansetron base, and dolasetron and pharmaceutically acceptable salts and solvates and prodrugs thereof, in an amount per unit form equivalent to 25 mg to 200 mg of dolasetron mesylate; aprepitant and pharmaceutically acceptable salt and solvates and prodrugs thereof, in an amount per unit form equivalent to from 10 mg to 250 mg of aprepitant base, and rolapitant and pharmaceutically acceptable salts and solvates and prodrugs thereof, in an amount per unit form equivalent to 15 mg to 270 mg of rolapitant base; and said dopamine agonist is pramipexole or a pharmaceutically acceptable salt thereof, in an amount per unit form equivalent to from 1.5 mg to 45 mg, from 1.5 mg to 25 mg or from 1.5 mg to 20 mg, preferably from more than 4.5 mg to 45 mg, normally from more than 4.5 mg to 25 mg or from more than 15 mg to 25 mg or from more than 20 mg to 25 mg, of pramipexole dihydrochloride monohydrate.

According to the above second and third embodiment of this fourth aspect of the invention, the pramipexole amounts per unit form and daily doses are those described in “The DA-agonist Component (b)” section. Specifically, in the second and third embodiments of this section, the pramipexole amount (in pramipexole dihydrochloride monohydrate) per unit form is in a range selected from the group consisting of from 0.125 mg to 45 mg, from 0.375 mg to 45 mg, form 1.5 mg to 45 mg, from 1.6 mg to 45 mg, from 1.625 mg to 45 mg, from 3 mg to 45 mg, from more than 4.5 mg to 45 mg, from 4.8 to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg and from 15 mg to 45 mg. Normally said range is from 15 mg to 25 mg or from more than 20 mg to 25 mg.

The above amounts per unit form include low amounts per unit form to be used during the titration period. During the effective treatment of a PMND such as PD or a PD-related disorder selected from the group consisting of MSA, DLB, LBD, DDS, FTLD, PSP, and CBD, composition comprising more than 4.5 mg to 45 mg, normally from more than 4.5 mg to 35 mg or from more than 4.5 mg to 30 mg, normally from 15 mg to 25 mg, may be used for at least arresting or slowing the progression of the disease.

In particular, for its use for the treatment of MSA, DLB, LBD, RLS, DDS, FTLD, PSP, and CBD, pramipexole or pharmaceutically acceptable salt or solvate thereof is administered to a patient in need of said treatment at a daily dose equivalent to from 0.125 mg to 45 mg, normally from 0.375 mg to 45 mg, from more than 4.5 mg to 45 mg, from 4.8 mg to 45 mg, from more than 6 mg to 45 mg, from 6.5 mg to 45 mg, from 7.25 mg to 45 mg, from 7.5 mg to 45 mg, from 10 mg to 45 mg, from 13 mg to 45 mg, from 14.5 mg to 45 mg; and from 15 mg to 45 mg, normally from 15 mg to 25 mg or from more than 20 mg to 25 mg of pramipexole dihydrochloride monohydrate.

In said combination, including fixed-dose combinations with said AEsI, in particular with at least one of the above (A)-(C)AEsIs, in a pharmaceutical composition in dosage unit form, pramipexole or a pharmaceutically acceptable salt thereof, said pramipexole or pharmaceutically acceptable salt or solvate thereof is normally present, in said pharmaceutical composition, in an amount per unit form equivalent to from 0.125 mg to 10 mg up to from 7.5 mg to 10 mg of pramipexole dihydrochloride monohydrate, in admixture with a pharmaceutical carrier or vehicle for an immediate-release formulation. For the administration of pramipexole at higher doses, said amount per IR-unit form will be equivalent to from 1.5 mg to 22.5 mg, from 1.6 mg to 22.5 mg, from more than 4.5 mg to 22.5 or from 7.5 mg to 22.5 mg, normally from 1.5 mg to 20 mg, of pramipexole dihydrochloride monohydrate.

Said pramipexole or pharmaceutically acceptable salt or solvate thereof will be present, in said pharmaceutical composition, in an amount per unit form equivalent to 1.5 mg to 45 mg or from more than 4.5 mg to 20 mg up to from 15 mg to 20 mg, of pramipexole dihydrochloride monohydrate, in admixture with a pharmaceutical carrier or vehicle for an extended-release formulation. For the administration of pramipexole at higher doses, said amount per ER-unit form will be equivalent to from more than 4.5 mg to 45 mg, up to or from 15 mg to 45 mg, normally from 15 mg to 25 mg or from more than 20 mg to 25 mg of pramipexole dihydrochloride monohydrate.

Pramipexole, in a pharmaceutical composition in dosage unit form, is in some cases administered to a patient suffering from MSA, DLB, LBD, DDS, FTLD, PSP, or CBD at a daily dose equivalent to from 0.375 mg to 20-21 mg, from 1.6 mg to 20-21 mg, from 1.625 mg to 20-21 mg, from 3 mg to 20-21 mg, from more than 4.5 mg to 20-21 mg, from 4.8 mg to 20-21 mg, from more than 6 mg to 20-21 mg, from 6.5 mg to 20-21 mg, from 10 mg to 20-21 mg, from 13.5 mg to 20-21 mg, from 14.5 mg to 45 mg; and from 15 mg to 20-21 mg, in particular from 1.5 mg to 20 mg, of pramipexole dihydrochloride monohydrate.

According to a fourth embodiment of this fourth aspect, the invention provides a pharmaceutical composition for use in the treatment of RLS in a patient in need of said treatment, comprising a pharmaceutical carrier or vehicle and a fixed-dose combination of at least one AEsI selected from the group consisting of 5HT3-antagonists and NK1-antagonists and a DA-agonist selected from the group consisting of pramipexole and pharmaceutically acceptable salts and solvates thereof, in an amount equivalent to from 0.125 mg to 6 mg or from 0.125 mg to 1 mg, normally from 0.125 mg to 0.75 mg or from 0.125 mg to 0.50 mg of pramipexole dihydrochloride monohydrate.

Advantageously, in this composition, said at least one AEsI is selected from the group consisting of ondansetron and pharmaceutically acceptable salt and solvates and prodrugs thereof, in an amount per unit form equivalent to from 2 mg to 32 mg of ondansetron base; dolasetron and pharmaceutically acceptable salts and solvates and prodrugs thereof, in an amount per unit form equivalent to 25 mg to 200 mg of dolasetron mesylate; palonosetron and pharmaceutically acceptable salts and solvates and prodrugs thereof, in an amount per unit form equivalent to from 0.25 mg to 0.5 mg of palonosetron base; aprepitant and pharmaceutically acceptable salts and solvates and prodrugs thereof, in an amount per unit form and at a daily dose equivalent to from 10 mg to 250 mg of aprepitant base; fosaprepitant and pharmaceutically acceptable salts and solvates and prodrugs thereof, in an amount per unit form equivalent to from 10 mg to 250 mg of aprepitant base; rolapitant and pharmaceutically acceptable salts and solvates and prodrugs thereof, in an amount per unit form equivalent to from 15 mg to 270 mg of rolapitant base; netupitant and pharmaceutically acceptable salts and solvates and prodrugs thereof, in an amount per unit form equivalent to from 300 mg to 600 mg of netupitant base.

Advantageously, in this composition, said at least one AEsI is selected from group consisting of ondansetron and pharmaceutically acceptable salt and solvates and prodrugs thereof, in an amount per unit form equivalent to from 2 mg to 8 mg of ondansetron base, and dolasetron and pharmaceutically acceptable salts and solvates and prodrugs thereof, in an amount per unit form equivalent to 25 mg to 100 mg of dolasetron mesylate; aprepitant and pharmaceutically acceptable salt and solvates and prodrugs thereof, in an amount per unit form equivalent to from 10 mg to 40 mg of aprepitant base, and rolapitant and pharmaceutically acceptable salts and solvates and prodrugs thereof, in an amount per unit form equivalent to 15 mg to 60 mg of rolapitant base.

### The Formulations

For the intended use in the treatment of PD and PD-related disorders in combination with a DA-agonist, the AEsI is formulated in a pharmaceutical composition, wherein said AEsI is in admixture with a pharmaceutical carrier or vehicle. For said treatment, also the DA-agonist is formulated in a pharmaceutical composition, wherein said DA-agonist is in admixture with a pharmaceutical carrier or vehicle.

The dosage, i.e. the amount of active ingredient in a single dose (amount per unit form) to be administered to the patient, can vary widely depending on the age, weight, and the health condition of the patient. This dosage includes the administration of a dose from 1 μg to 600 mg, normally from 1 mg to 600 mg or from 1 mg to 300 mg, according to the potency of each AEsI and the age of the patient, and an amount of DA-agonist that is from 0.001 mg to 200 mg, according to the conditions and the age of the patient, from one to five times a day by, according to the strength of the doses of the each of the active ingredients. The pharmaceutical compositions are formulated in admixture with a pharmaceutical carrier or vehicle for any administration route.

In the pharmaceutical compositions of the present invention for oral, subcutaneous, transcutaneous, GI infusion, sublingual, intranasal, intravenous (including infusion), transdermal, rectal or topical administration, the active ingredients are preferably administered in the form of dosage units, in admixture with the classic pharmaceutical carriers or vehicles.

The above pharmaceutical compositions are formulated, normally in unit forms in admixture with a pharmaceutical carrier or vehicle for any administration route. In the pharmaceutical compositions of the present invention for oral, subcutaneous, transcutaneous, GI infusion, sublingual, intranasal, intraperitoneal, intramuscular, intravenous (including infusion), transdermal, rectal or topical administration, the active ingredients are preferably administered in the form of dosage units, in admixture with the classic pharmaceutical carriers or vehicles, as set forth above.

These unit forms are manufactured according to conventional technologies.

Particularly advantageous are the formulations in the form of tablets, multi-score tablets, multi-layer tablets, coated tables, orally disintegrating tablets, extended release tablets, hard or soft capsules, multi-compartment capsules, extended-release capsules, suppositories for rectal administration, patches for transdermal administration, liquid oral solutions, syrups or suspensions in a predetermined unit form, apparatus for intravenous infusion, and vials for the intravenous or subcutaneous administration. Taste-masking agents such as sucrose, aspartame, saccharin sodium, spices such as licorice spice and spice mixtures may be added to oral formulations.

The pharmaceutical compositions may be formulated in oral unit forms such as tablets or gelatin capsules wherein the AEsI or the DA-agonist or both the active ingredients are in admixture with a carrier or vehicle that may include a diluent, such as cellulose, dextrose, lactose, mannitol, sorbitol or sucrose; a lubricant, such as stearic acid, calcium or magnesium stearate, polyethylene glycol, silica, or talc; and if needed, a binder such as magnesium aluminum silicate, gelatin, methylcellulose, sodium carboxymethylcellulose, or polyvinylpyrrolidone.

Said oral unit forms may be tablets coated with sucrose or with various polymers for an immediate release.

Alternatively, the tablets can be manufactured by using carriers such as appropriate materials, to have a prolonged or delayed activity by progressively releasing a predetermined quantity of AEsI, or DA-agonist, or of both the active ingredients. The oral formulations can also be in form of capsules allowing the extended release of the AEsI, or of DA-agonist, or of both the active ingredients. Carriers and vehicles for ER-unit forms include retardant materials such as acrylic and methacrylic acid polymers and copolymers; the aforementioned cellulose derivatives such as hydroxypropylmethylcellulose, hydroxyethylcellulose, hydroxypropylethylcellulose, hydroxypropylcellulose, methylcellulose, ethylcellulose, or sodium carboxymethylcellulose; gums; waxes; glycerides or aliphatic alcohols or a mixture thereof.

Formulation for oral route include syrups, solutions or suspension (for example to be administered in drops), orally dispersible tablets and chewable tablets may also comprise sweeteners, lubricants, taste-masking agents, binders, and coloring agents.

Suppositories are manufactured by using a suppository base such as cocoa butter, poloxamers combined with solvents such as. polyethylene glycols (for example polyethylene glycol 400, polyethylene glycol 1000 and polyethylene glycol 3350), propylene glycol, butylhydroxyanisole, tartaric acid, sorbitan monooleate, triglycerides or semi-synthetic glycerides according to conventional technologies.

For its use in combination with a DA-agonist, in particular with pramipexole, ondansetron may be formulated in a coated tablet, in an orally disintegrable tablet, in a syrup, in injectable solution for intravenous, subcutaneous or intramuscular use, or in a suppository for rectal use.

For its use in combination with a DA-agonist, in particular with pramipexole, domperidone may be manufactured in compositions for oral administration, such as tablets, film-coated tablets, chewable tablets, orally dispersible tablets, effervescent granules, or in oral solution or suspension to be administered as a syrup or in drops. Taste-masking agents, for example sucrose, aspartame, saccharin sodium, spices such as licorice spice and spice mixtures may be added to the formulation. Domperidone may also be formulated in a suppository for rectal use.

The pharmaceutical compositions of the present invention are in unit form formulated with the classic excipients suitable for different ways of administration, as described above. Said unit forms are manufactured according to conventional technologies allowing, for example, the formulation of the AEsI in an IR-form and of the DA-agonist in ER-form in the same unit-form. Particularly advantageous are the formulations in the form of tablets, multi-score tablets, multi-layer tablets, coated tables, orally disintegrating tablets, extended release tablets, hard or soft capsules, multi-compartment capsules, extended-release capsules, patches for transdermal administration, liquid oral solutions, syrups or suspensions in a predetermined unit form, and vials for the intravenous or subcutaneous administration.

Said oral unit forms may also be tablets or capsules wherein one of the active ingredient is in an IR-formulation and the other one is in an ER-formulation. For example said unit form comprises aprepitant or rolapitant in an IR-formulation and pramipexole dihydrochloride monohydrate in an ER-formulation, each at the amount per unit form as described above.

Similarly, a tablet or capsule may comprise

as AEsI Component (a), the combination of palonosetron hydrochloride, in an amount equivalent to 0.5 mg of palonosetron base, and netupitant, in an amount of 300 mg, in an IR-formulation; and

as DA-agonist Component (b), pramipexole dihydrochloride monohydrate in an amount of from 0.375 mg to 45 mg, normally from 1.5 mg to 20 mg, in an ER-formulation.

The pharmaceutical compositions may also be formulated in TDDS, such as a patch formulation wherein the active ingredient or the mixture of the active ingredients may comprise adjuvants such as D-sorbitol, gelatin, kaolin, methyl paraben, polysorbate 80, propylene glycol, propyl paraben, povidone, sodium carboxymethylcellulose, sodium polyacrylate, tartaric acid, titanium dioxide, and purified water. A patch formulation may also contain skin permeability enhancer such as lactate esters (e.g., lauryl lactate), triacetin or diethylene glycol monoethyl ether.

“Transdermal drug delivery system” provides transdermal delivery using transdermal drug formulations and transdermal patches incorporating such transdermal drug formulations. For example, the transdermal drug delivery system may include a composition in form of a patch, a cream, a gel, a lotion or a paste comprising a 5HT3-antagonist (such as ondansetron). Examples of transdermal formulations may include, but are not limited, to those as described in U.S. Pat. No. 6,562,368, a transdermal gel formulation as described in U.S. Pat. Nos. 7,029,694; 7,179,483; 8,241,662 and US 2009/0018190, a transdermal or transmucosal pharmaceutical formulation, that can be utilized for topical or transdermal application, such that solutions, creams, lotions, sprays, ointment, gels, aerosols and patch drug deliveries as described in WO 2005/039531, US2007/022379, US 2010/0216880, US 2014/0037713 and U.S. Pat. No. 8,652,491, a transdermal absorption preparation as described in WO2013/061969 and US 2014/0271796, the disclosures of which are herein incorporated by reference in their entirety. The transdermal patches may also include, but are not limited to, a patch pump having an in-dwelling rigid catheter with flexible features and/or a flexible catheter attachment as described in U.S. Pat. No. 9,782,536, a selectively activatable patch pump as described in U.S. Pat. No. 9,724,462, a patch pump attached to a wireless communication system as described in U.S. Pat. No. 9,623,173, a conformable patch pump as described in U.S. Pat. No. 9,616,171, an infusion pump as described in U.S. Pat. No. 8,915,879, a portable infusion drug delivery as described in U.S. Pat. No. 8,480,649, a micropump as described in U.S. Pat. No. 8,282,366, and a patch pump as described in U.S. Pat. No. 7,828,771; the disclosures of which are herein incorporated by reference in their entirety. Other transdermal patches may include, but are not limited to, a patch in which oxybutynin is incorporated in an adhesive agent layer composition comprises the acrylic-based polymer as the adhesive base agent, and the acrylic-based polymer is a copolymer of polymethyl methacrylate with a polyacrylate as described in U.S. Pat. No. 8,802,134, a patch consisting of a support layer and of an adhesive agent layer arranged on the at least one surface of the support layer as described in U.S. Pat. No. 8,877,235, a patch using a monoglyceride or a mixture of monoglycerides of fatty acids as skin permeation-enhancer as described in U.S. Pat. Nos. 5,441,740 and 5,500,222, a patch for using a monoglyceride or a mixture of monoglycerides plus a lactate ester as skin permeation-enhancer as described in U.S. Pat. Nos. 5,686,097; 5,747,065; 5,750,137 and 5,900,250, a patch with a non-rate controlling tie layer on the skin-proximal surface of the reservoir, not affecting the drug release as described in U.S. Pat. Nos. 5,614,211 and 5,635,203, a patch using triacetin as permeation enhancer as described in U.S. Pat. Nos. 5,212,199, 5,227,169, 5,601,839 and 5,834,010, a patch with a matrix mass in the form of a layer which is self-adhesive, and in which the matrix mass consists of ammonium-group-containing (meth)acrylate copolymers as described in U.S. Pat. No. 6,555,129, a transdermal patch as described in U.S. Pat. Nos. 6,743,441; 7,081,249; 7,081,250; 7,081,251; 7,081,252 and 7,087,241; the disclosures of which are herein incorporated by reference in their entirety. Preferably, the transdermal drug delivery system is a patch, a patch pump, an infusion pump, or a micropump.

For example, a TDDS for the treatment of PD or a related disorder comprises ondansetron base, in an amount releasing fro 4 mg to 16 mg of ondansetron; and rotigotine base, in an amount releasing from 2 mg to 24 mg of rotigotine base.

For example, in the treatment of a PMND such as AD, PD, MSA, DDS, FTLD, PSP, or CBD in a patient in need of said treatment, the AEsI is preferably at least one AESI selected from the group consisting of domperidone base, domperidone maleate or domperidone succinate (1:1) in a dosage (in domperidone base) ranging from 2 mg to 120 mg, normally from 2 mg to 40 mg; ondansetron hydrochloride dihydrate, in a dosage ranging from 2 mg to 32 mg, from 4 mg to 32 mg, or from 4 mg to 16 mg (in ondansetron base); aprepitant, in a dosage ranging from 10 mg to 250 mg; and rolapitant, in a dosage ranging from 15 mg to 270 mg and the DA-agonist is preferably pramipexole dihydrochloride monohydrate, in a dosage ranging from 0.125 mg to 42 mg, preferably from 1.5 mg to 42 mg, normally from 1.5 mg to 20 mg.

In the above pharmaceutical compositions, an advantageous NK1-antagonist active ingredient is aprepitant, fosaprepitant, rolapitant, netupitant-300/palonosetron-0.5, or fosnetupitant-235/palonosetron-0.25, and the DA-agonist preferably is pramipexole base or a pharmaceutically acceptable salt or solvate thereof.

In the case of pediatric or obese patients, the NK1-antagonist daily dose may be decided on the basis of the body weight. Thus, for example, aprepitant may be administered at a daily dose of 0.16 mg/kg to 4.2 mg/kg and rolapitant may be administered at a daily dose of 0.25 mg/kg to 4.5 mg/kg.

### Kits

The present invention also provides a kit or package containing a medicament, a pharmaceutical combination, or a pharmaceutical composition as described herein, accompanied by instructions for use of the same in the treatment of a PMND in a patient in need thereof.

In one embodiment, a kit of the present invention is a kit comprising Unit Form Component (a), wherein the AEsI is in admixture with a pharmaceutical carrier or vehicle; and instructions for use of the same for treatment of a PMND in a patient in need thereof, in combination with pramipexole.

In another embodiment, a kit of the present invention is a kit comprising pharmaceutical composition (a) comprising at least one of AESI and a pharmaceutical composition (b) comprising pramipexole; and instructions for use of the same for treatment of a PMND in a patient in need thereof.

### Example 1

The ability of a 5HT3-antagonist and of a NK1-antagonist for preventing the adverse effects of a DA-agonist in humans was tested.

To be enrolled in the study, participants the following inclusion/exclusion key criteria:

### Key Inclusion Criteria

1. Male and female subjects aged 20-45 years old both ages included.
2. Females of childbearing potential must agree to be abstinent or else use any two of the following medically acceptable forms of contraception from the Screening Period through 14 days after the study Exit Visit: condom with spermicidal jelly, diaphragm or cervical cap with spermicidal jelly, or intrauterine device (IUD). A female whose male partner has had a vasectomy must agree to use one additional form of medically acceptable contraception. Subjects must agree to practice the above birth control methods for 14 days after the final visit as a safety precaution.
3. Females of non-childbearing potential, defined as surgically sterile (status post-hysterectomy, bilateral oophorectomy, or bilateral tubal ligation) or post-menopausal for at least 12 months, do not require contraception during the study. The reason must be documented in the source documents.
4. Males with female partners of childbearing potential must agree to use a highly effective, medically acceptable form of contraception from the Screening Period through 14 days after the study Exit Visit. Males with female partners of childbearing potential who themselves are surgically sterile (status post vasectomy) must agree to use condoms with spermicide over the same period of time. Male subjects must agree to practice the above birth control methods for 14 days after the final visit as a safety precaution.
5. Subjects must be in good health as determined by their medical history including personal and family psychiatric history and results of physical examination, electrocardiogram (ECG), vital signs, and laboratory tests. A subject with a medical abnormality may be included only if the investigator or designee considers that the abnormality will not introduce significant additional risk to the subject's health or interfere with study objectives.
6. Subjects must be able to clearly and reliably communicate changes in their medical condition.
7. Subjects with a body mass index (BMI) between 19.0 and 32.0 kg/m² (both inclusive).
8. Subjects able to swallow multiple pills or capsules simultaneously.
9. Subjects must have signed an informed consent form indicating that they understand the purpose of and procedures required for the study and are willing to participate in the study and comply with the study procedures and restrictions.

### Key Exclusion Criteria:

The criteria for exclusion of a subject from enrollment in the study were as follows:
1. Any clinically relevant acute or chronic diseases, which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of the study drugs.
3. History of substance abuse, known drug addiction, or positive test for drugs of abuse or alcohol.
4. History of drug or other significant allergy.
5. Known hypersensitivity to pramipexole, or to ondansetron or similar serotonin receptor antagonists, or to aprepitant or similar Substance P/NK1 receptor antagonists.
5. History of and/or current QT interval prolongation, congenital long QT syndrome, electrolyte abnormalities (e.g., hypokalemia or hypomagnesemia), congestive heart failure, bradyarrhythmias or other medicinal products that lead to QT prolongation or 1st degree AV block at Screening, Day−1, or pre-dose, ≥450 QTcF for males and ≥470 QTcF for females.
7. Treatment with centrally active drugs or antiemetics, within 1 months of study entry.
8. Tobacco or nicotine users (except subjects who stopped using tobacco or nicotine 1 year or more before enrollment in the study).
9. Excessive daily consumption of xanthines containing drinks (i.e. >500 mg/day of caffeine).
10. Subjects unwilling to curtail prolonged intensive physical exercise during the study conduct (from the Screening visit until the last dose of study drug).
11. Positive test result for hepatitis B surface antigen, hepatitis C antibody.
12. Positive test result for HIV 1 or 2 serology.
13. Likely to need any medical or dental treatment during the study period.
14. Use of any prescription or over-the-counter medication within 14 days prior to admission on Day-1. In addition, any medications with central effects are prohibited for a period equal to 5 times the drug half-life prior to admission (Day −1), should this period be longer than 14 days.
15. Subjects unlikely to co-operate during the study, and/or be questionably compliant in the opinion of the investigator.
16. Subjects unable to be contacted in case of an emergency.
17. Intake of an investigational drug within 30 days of study entry.
18. Show evidence of suicidal ideation within the last 6 months as assessed by the C-SSRS (Columbia Suicide Severity Rating Scale) at Screening.

Following enrollment in the study, participants received single increasing oral doses of pramipexole given once each day in the morning (Period 1 of the study). The starting dose of pramipexole was 0.5 mg and the dose was increased daily by 0.5 mg increments to the maximum protocol allowable dose of 6 mg as tolerated. Once a subject had reached his/her first intolerable dose (FID-1), upward dose escalation was discontinued. First intolerable dose (FID) was defined as:
One (1) episode of vomiting; or
Two (2) episodes of retching, or
One (1) episode of severe nausea (Grade 3; defined as nausea interfering with activities of daily living or inadequate oral caloric or fluid intake; tube feeding, total parenteral nutrition or hospitalization indicated) lasting more than 1 hour, or
Three (3) consecutive episodes at every 4 hour ratings of moderate nausea (Grade 2; defined as subjectively symptomatic, but not interfering with activities of daily living), or
One (1) episode of moderate diarrhea (Grade 2; defined as 4-6 stools more than at baseline).

When a subject reached FID-1 on pramipexole alone, they were washed out for at least 5 days, and then entered Study Period 2 during which they received single daily oral doses of pramipexole (using the same titration schedule as in Period 1, i.e., starting at 0.5 mg and increasing daily in 0.5 mg increments to a maximum of 6 mg) in combination with an oral antiemetic, either ondansetron (8 mg) in Period 2 or aprepitant (80 mg) in Period 3 until subjects again reached an intolerable dose defined as above. The FID on oral pramipexole plus oral ondansetron or aprepitant was referred to as FID-2.

On each study day, subjects were followed up for up to 8 hours following drug administration for AEs, vital signs, ECGs. In addition, a laboratory panel was taken at screening and at the end of the study.

Four subjects were enrolled in the study. The following Table 1 summarizes their demographic characteristics.

TABLE 1
Demographic Characteristics of Subjects Enrolled in the Study

| | | | Baseline Weight |
|---|---|---|---|
| Subject ID | Gender | Age (years) | (kg) |
| 1001 (019) | Female | 40 | 76.4 kg |
| 1006 (001) | Male | 41 | 99.1 kg |
| 1007 (004) | Male | 38 | 64.9 kg |
| 1008 (008) | Male | 39 | 81.8 kg |

All subjects reached FID-1 (pramipexole alone) during the study. The dose limiting toxicity was a gastro-intestinal adverse event in all 4 subjects.

During Period 2 of the study, 3 of the 4 subjects tolerated the highest pramipexole dose allowed per protocol of 6 mg given in combination with ondansetron (Table 2). One individual reached FID-2 (due to severe nausea) at a pramipexole dose of 1.0 mg compared with FID-1 of 0.5 mg when pramipexole was administered alone. The mean MTD of pramipexole alone in the 4 subjects evaluated was 2.1±0.66 compared with 4.6±1.3 when pramipexole was give with ondansetron (p<0.05).

During Period 3, all 4 subjects tolerated the maximum pramipexole dose of 6 mg allowed by the protocol and therefore none of them reach FID-3 (pramipexole with aprepitant). In other words, concomitant administration of aprepitant with pramipexole prevented the occurrence of dose-limiting adverse events (in these cases all were gastro-intestinal) associated with high doses of pramipexole. Table 2 lists for each subject the values for FID-1 (on pramipexole alone) and FID-3 (on pramipexole+aprepitant).

TABLE 2
Listing of First Intolerable Doses (FID) values

| | | FID-1 | | |
|---|---|---|---|---|
| | | Dose | FID-2 | FID-3 |
| | FID-1 | Limiting | Pramipexole | Pramipexole |
| Subject | (Pramipexole | Adverse | + | + |
| ID | alone) | Event | Ondansetron | Aprepitant |
| 1001 | 2.5 mg | GI upset | >6.0 mg | >6.0 mg |
| 1006 | 0.5 mg | Moderate | 1.0 mg | >6.0 mg |
| | | nausea | | |
| 1007 | 4.5 mg | Severe | >6.0 mg | >6.0 mg |
| | | nausea | | |
| 1008 | 1.5 mg | Vomiting | >6.0 mg | >6.0 mg |

FID—First intolerable dose
Subject 1006 dose limiting adverse event with pramipexole plus ondansetron was severe nausea

As illustrated in Table 3 below, the MTD of pramipexole (given with aprepitant) during Period 3 was higher than the MTD of pramipexole (given alone) during Period 1 in all subjects; in 3 of the 4 subjects, MTD-2 increased by more than 3-fold. The mean MTD of pramipexole given alone in these subjects was 2.1±0.66 compared with 6.0±0.0 when pramipexole was given with aprepitant (p<0.05).

Taken together, the results showed that the co-administration of ondansetron or of aprepitant with pramipexole substantially reduced all the dose-limiting adverse effects occurring with pramipexole alone, thus indicating that an AEsI of the 5HT3-antagonist or NK1-antagonist class enables the administration to a human being of high pramipexole doses that are not tolerated when the DA agonist is administered alone.

TABLE 3
Listing of Maximum Tolerated Doses (MTD)

| | Maximal | Maximal | Maximal | | |
|---|---|---|---|---|---|
| | Tolerated | Tolerated | Tolerated | | |
| | Dose | Dose | Dose | | |
| | MTD-1 | MTD-2 | MTD-3 | Ratio | Ratio |
| Subject | (Pramipexole | Pramipexole + | Pramipexole + | MTD2/ | MTD3/ |
| ID | alone) | Ondansetron | Aprepitant | MTD1 | MTD1 |
| 1001 | 2.0 mg | ≥6.0 mg | ≥6.0 mg | ≥3.0 | ≥3.0 |
| 1006 | <0.5 | 0.5 mg | ≥6.0 mg | >1 | ≥6 |
| | (not | | | | |
| | tolerated | | | | |
| | at 0.5 mg) | | | | |
| 1007 | 4.0 mg | ≥6.0 mg | ≥6.0 mg | ≥1.5 | ≥1.5 |
| 1008 | 1.0 mg | ≥6.0 mg | ≥6.0 mg | ≥6.0 | ≥6.0 |

MTD = Maximum Tolerated Dose

Accordingly, the co-administration of a 5HT3-antagonist and/or of a NK1-antagonist with pramipexole substantially blocks the dose-limiting adverse effects of pramipexole when given alone, thus enabling doses of a DA agonist to be to be safely and tolerably increased several folds, and thereby allowing the greater antiparkinsonian efficacy of higher agonist doses to be fully realized.

It has thus been shown that the co-administration of an AEsI of the 5HT3-antagonist and/or NK1-antagonist class will enable the safe and tolerable administration of higher dose of a DA agonist such a pramipexole to a PD or RLS or similar patient and thereby confer a far greater degree of symptomatic relief than achieved when the DA agonist is given alone. All PD patients as well as those with other diseases treated with drugs of this type become the beneficiaries of the use of characteristically longer acting drugs that carry a lesser risk of inducing or exacerbating MC. This risk reduction has been related to the more continuous and thus physiologic normalization of brain dopaminergic transmission afforded by DA agonists having a relatively long half-life. For example, LD has a plasma half-life of only about 1.5 hours, while many DA agonists have a half-life extending from about 6-8 hours (e.g., ropinirole and pramipexole IR), to 9-12 hours (pramipexole ER for once per day dosing) and beyond (cabergoline at about 65 hours). DA agonists with half-lives exceeding levodopa have been reported to bring both palliative and prophylactic benefit to those with PD like disorders: they appear to ameliorate existing MC and to delay the onset and reduce the severity of pending MC. Thus, the use of relatively high doses of long acting DA agonists, such s pramipexole ER as enabled by the co-administration of a suitable antiemetic, in the treatment of PD type disorders, in particular RLS, DDS, FTLD, PSP, and CBD potentially confers both enhanced efficacy (greater symptomatic relief) and greater safety (reduce risk of MC) to those suffering from these incurable, ineluctably progressive disorders.

### Example 2

The ability of domperidone to prevent the gastro-intestinal (GI) adverse effects (AEs) of pramipexole in humans is tested.

A Phase I study is conducted in subjects receiving a single oral dose of pramipexole dihydrochloride monohydrate (“pramipexole”) with or without a single oral dose of domperidone base (“domperidone”). The study is a single center study.

The objective of the study is to demonstrate that domperidone safely attenuates the gastro-intestinal side effects of pramipexole given in doses equivalent to or higher than those approved in the treatment of Parkinson's Disease or shown in clinical trials to be effective in the treatment of depression.

To be enrolled in the study, participants meet the following inclusion/exclusion key criteria:

### Key Inclusion Criteria

2. Male and female subjects aged 20-45 years old both ages are included.
2. Females of childbearing potential must agree to be abstinent or else use any two of the following medically acceptable forms of contraception from the Screening Period through 14 days after the study Exit Visit: condom with spermicidal jelly, diaphragm or cervical cap with spermicidal jelly, or intrauterine device (IUD). A female whose male partner has had a vasectomy must agree to use one additional form of medically acceptable contraception. Subjects must agree to practice the above birth control methods for 14 days after the final visit as a safety precaution.
3. Females of non-childbearing potential, defined as surgically sterile (status post-hysterectomy, bilateral oophorectomy, or bilateral tubal ligation) or post-menopausal for at least 12 months, do not require contraception during the study. The reason must be documented in the source documents.
4. Males with female partners of childbearing potential must agree to use a highly effective, medically acceptable form of contraception from the Screening Period through 14 days after the study Exit Visit. Males with female partners of childbearing potential who themselves are surgically sterile (status post vasectomy) must agree to use condoms with spermicide over the same period of time. Male subjects must agree to practice the above birth control methods for 14 days after the final visit as a safety precaution.
5. Subjects must be in good health as determined by their medical history including personal and family psychiatric history and results of physical examination, electrocardiogram (ECG), vital signs, and laboratory tests. A subject with a medical abnormality may be included only if the investigator or designee considers that the abnormality will not introduce significant additional risk to the subject's health or interfere with study objectives. 6. Subjects must be able to clearly and reliably communicate changes in their medical condition.
7. Subjects with a body mass index (BMI) between 19.0 and 32.0 kg/m² (both inclusive).
8. Subjects able to swallow multiple pills or capsules simultaneously.
9. Subjects must sign an informed consent form indicating that they understand the purpose of and procedures required for the study and are willing to participate in the study and comply with the study procedures and restrictions.

### Key Exclusion Criteria:

The criteria for exclusion of a subject from enrollment in the study are as follows:
1. Any clinically relevant acute or chronic diseases which could interfere with the subjects' safety during the trial, expose them to undue risk, or interfere with the study objectives.
2. History or presence of gastrointestinal, hepatic, or renal disease or other condition known to interfere with the absorption, distribution, metabolism or excretion of the study drugs.
3. History of substance abuse, known drug addiction, or positive test for drugs of abuse or alcohol.
4. History of drug or other significant allergy.
5. Known hypersensitivity to pramipexole, or to domperidone or similar dopamine receptor antagonists.
5. History of and/or current QT interval prolongation, congenital long QT syndrome, electrolyte abnormalities (e.g., hypokalemia or hypomagnesemia), congestive heart failure, bradyarrhythmias or other medicinal products that lead to QT prolongation or 1st degree AV block at Screening, Day−1, or pre-dose, ≥450 QTcF for males and ≥470 QTcF for females.
7. Treatment with centrally active drugs or antiemetics, within 1 month of study entry.
8. Tobacco or nicotine users (except subjects who stopped using tobacco or nicotine 1 year or more before enrollment in the study).
9. Excessive daily consumption of xanthines containing drinks (i.e. >500 mg/day of caffeine).
10. Subjects unwilling to curtail prolonged intensive physical exercise during the study conduct (from the Screening visit until the last dose of study drug).
11. Positive test result for hepatitis B surface antigen, hepatitis C antibody.
12. Positive test result for HIV 1 or 2 serology.
13. Likely to need any medical or dental treatment during the study period.
14. Use of any prescription or over-the-counter medication within 14 days prior to admission on Day-1. In addition, any medications with central effects are prohibited for a period equal to 5 times the drug half-life prior to admission (Day −1), should this period be longer than 14 days.
15. Subjects unlikely to co-operate during the study, and/or be questionably compliant in the opinion of the investigator.
16. Subjects unable to be contacted in case of an emergency.
17. Intake of an investigational drug within 30 days of study entry.
18. Show evidence of suicidal ideation within the last 6 months as assessed by the C-SSRS (Columbia Suicide Severity Rating Scale) at Screening.

Following enrollment in the study, participants will receive single increasing oral doses of pramipexole given once daily in the morning (Period 1 of the study). The starting dose of pramipexole is 0.5 mg and the dose increases daily by 0.5 mg increments. Once a subject reaches his/her first intolerable dose (FID-1), upward dose escalation is discontinued. First intolerable dose (FID) is defined as:
One (1) episode of vomiting; or
Two (2) episodes of retching, or
One (1) episode of severe nausea (Grade 3; defined as nausea interfering with activities of daily living or inadequate oral caloric or fluid intake; tube feeding, total parenteral nutrition or hospitalization indicated) lasting more than 1 hour, or
Three (3) consecutive episodes at every 4 hour ratings of moderate nausea (Grade 2; defined as subjectively symptomatic, but not interfering with activities of daily living), or
One (1) episode of moderate diarrhea (Grade 2; defined as 4-6 stools more than at baseline).

When a subject reaches FID-1 on pramipexole alone, the subject is washed out for at least 5 days, and then enters Period 2 of the study during which the subject receives single daily oral doses of pramipexole starting at 0.5 mg and titrated upward by 0.5 mg increments, together with oral domperidone (5 mg) until subject again reaches an intolerable dose defined as above. The FID on oral pramipexole plus oral domperidone is referred to as FID-2.

If a subject reaches FID-2 during Period 2 at the same or lower dose than FID-1, and providing the investigator judges there are no safety issues and the subject is consenting, the subject receives the same dose of pramipexole as the FID-2 dose together with a higher dose of oral domperidone (10 mg) on the next day and the protocol specifies that said subject should continue with the remainder of the dose titration with the higher dose of oral domperidone (10 mg) until they reach the intolerable dose (FID2+). All other provisions of the protocol remain unchanged. Assessments are the same as those planned for the dose escalation day.

On each study day, subjects are followed up for up to 8 hours following drug administration for AEs, vital signs, ECGs. In addition, a laboratory panel is taken at screening and at the end of the study.

Results show that the co-administration of domperidone with pramipexole attenuates dose-limiting gastro-intestinal adverse effects reported with pramipexole alone, thus showing that a domperidone enables the administration to a human being of pramipexole in doses otherwise non-tolerated when administering pramipexole alone.

### REFERENCES

Al-Mansoori et al. 2013: Al-Mansoori K M, Hasan M Y, Al-Hayani A, El-Agnaf M, “The role of α-synuclein in neurodegenerative diseases: from molecular pathways in disease to therapeutic approaches”. Curr Alzheimer Res 2013, July; 10(6): 559-568.
Braak et al. 2003: Braak H, Del Tredici K, Rub U, de Vos R A, Jansen Steur E N, Braak E. “Staging of brain pathology related to sporadic Parkinson's disease”. Neurobiol Aging. 2003 March-April; 24(2):197-211.
Bruni et al 2013: Bruni G, Maietta M, Scotti F, Maggi L, Bini M, Ferrari S, Capsoni D, Boiocchi M, Berbenni V, Milanese C, Girella A, Marini A. “Structure and properties of domperidone and its succinate salt” Acta Crystallogr B Struct Sci Cryst Eng Mater. 2013 August; 69(Pt 4):362-70. doi: 10.1107/52052519213016989. Epub 2013 Jul. 18.
Castillo-Carranza et al. 2018: Castillo-Carranza D L, Marcos J. Guerrero-Muñoz M J, Sengupta U, Gerson J E, Kayed R; “α-Synuclein Oligomers Induce a Unique Toxic Tau Strain”, Biol. Psychiatry 2018, January pii: S0006-3223(18)30034-9.
Chen J J 2011: Chen J J. “Pharmacologic safety concerns in Parkinson's disease: facts and insights”. Int J Neurosci 2011, 121 Suppl 2:45-52. doi: 10.3109/00207454.2011.620193.
Chen M et al. 2016: Chen M, Weiwei Yang W, Li Xin, X, Li Xuran, Wang P, Feng Yue F, Yang H, Chan P, and Yu S; “Age- and brain region-dependent α-synuclein oligomerization is attributed to alterations in intrinsic enzymes regulating α-synuclein phosphorylation in aging monkey brains”. Oncotarget 2016, Feb. 23; 7(8): 8466-8480.
Choi and Gandhi 2018: Choi M L, Gandhi S. “Crucial role of protein oligomerization in the pathogenesis of Alzheimer's and Parkinson's diseases”. FEBS J 2018, Jun. 20.
Choksi et al. 2014: Choksi D K, Roy B, Chatterjee S, Yusuff T, Bakhoum M. F, Sengupta U, Ambegaokar S, Kayed R, Jackson G R. “TDP-43 phosphorylation by casein kinase Iepsilon promotes oligomerization and enhances toxicity in vivo”. Hum Mol Genet 2014, 23, 1025-1035.
Chow et al. 2010: Chow V W, Savonenko A V, Melnikova T, Kim H, Price D L, Li T, Wong P C. “Modeling an anti-amyloid combination therapy for Alzheimer's disease”. Sci Transl Med 2010, Jan. 6; 2(13):13ra1. PubMed. 8 Jan. 2010.
Cline et al. 2018: Cline E N, Bicca M A, Viola K L, Klein W L “The Amyloid-β Oligomer Hypothesis: Beginning of the Third Decade”. J Alzheimer Dis. 2018; 64(s1):S567-S610.
Connolly and Lang 2014: Connolly B S, Lang A E, “Pharmacological treatment of Parkinson disease: a review”. JAMA 2014, Apr. 23-30; 311(16):1670-83).
Erro Aguirre et al. 2015: Erro Aguirre M E, Zelaya M V, Sánchez Ruiz de Gordoa J, Tuñón M T, Lanciego J L. “Midbrain catecholaminergic neurons co-express α-synuclein and tau in progressive supranuclear palsy”; Front Neuroanat. 2015, Mar. 11; 9:25. doi: 10.3389/fnana.2015.00025. eCollection 2015 (March).
Fang et al. 2014: Fang Y S, Tsai K J, Chang Y J, Kao P, Woods R, Kuo, P H, Wu C C, Liao J Y, Chou S C, Lin V, Jin L W, Yuan H S, Cheng I H, Tu P H, Chen Y R. “Full-length TDP-43 forms toxic amyloid oligomers that are present in frontotemporal lobar dementia-TDP patients”. Nat Commun 2014, 5, 4824.
Gandy et al. 2010: Gandy S, Simon A J, Steele J W, Lublin, A L, Lah, J J, Walker L C, Levey A I, Krafft G A, Levy E, Checler F, Glabe C, Bilker W., Abel T. Schmeidler J., Ehrlich M E. “Days-to-criterion as an indicator of toxicity associated with human Alzheimer amyloid-β oligomers”. Ann Neurol 2010, 68, 220-230.
Gerson and Kayed 2013: Gerson J E, Kayed R. “Formation and propagation of tau oligomeric seeds”. Front Neurol 2013, 4, 93.
Glabe 2006: Glabe C G. “Common mechanisms of amyloid oligomer pathogenesis in degenerative disease”. Neurobiol Aging 2006, 27, 570-575.
Jellinger 2008a: Jellinger K A. “A critical reappraisal of current staging of Lewy-related pathology in human brain; Acta Neuropathol 2008, July; 116(1):1-16.
Jellinger 2008b: Jellinger K A, “Neuropathological aspects of Alzheimer disease, Parkinson disease and frontotemporal dementia”; Neurodegener. Dis. 2008; 5(3-4): 118-121.
Marques and Outeiro 2012: Marques O, Outeiro T F. “Alpha-synuclein: from secretion to dysfunction and death”; Cell Death Dis 2012, Jul. 19; 3:e350. doi: 10.1038/cddis.2012.94.
Murphy et al. 2008: Murphy K E, Karaconji T, Hardman C D, Halliday G M. “Excessive dopamine neuron loss in progressive supranuclear palsy”. Movement Disorders 2008 23(4) March 15: 607-610.
Nilson et al. 2017: Nilson A N, English K C, Gerson J E, Barton Whittle T, Nicolas Crain C, Xue J, Sengupta U, Castillo-Carranza D L, Zhang W, Gupta P, Kayed R. “Tau Oligomers Associate with Inflammation in the Brain and Retina of Tauopathy Mice and in Neurodegenerative Diseases”. J Alzheimers Dis 2017, 55(3):1083-1099.
O'Regan et al. 2017: O'Regan G, DeSouza R M, Balestrino R, “Glucocerebrosidase Mutations in Parkinson Disease”. J Parkinson's Dis 2017, 7 411-422—doi 10.3233/JPD-171092.
Peeraully et al. 2012: Peeraully T, Tan E K. “Linking restless legs syndrome with Parkinson's disease: clinical, imaging and genetic evidence”. Transl Neurodegener. 2012; 1: 6. doi: 10.1186/2047-9158-1-6.
Petersen et al. 2013: Petersen R C, Aisen P, Boeve B F, Geda Y E, Ivnik R J, Knopman D S, Mielke M, Pankratz V S, Roberts R, Rocca W A, Weigand S, Weiner M, Wiste H, Jack C R. “Criteria for mild cognitive impairment due to Alzheimer's disease in the community”. Ann Neuro 2013, 74, 199-208.
Pittock et al. 2004: Pittock S J, Parrett T, Adler C H, Parisi J E, Dickson D W, J. Eric Ahlskog J E. “Neuropathology of primary restless leg syndrome: Absence of specific τ□ and a□synuclein pathology”. Movement Disorders 2004(June), 19(6) 695-699.
Poewe et al. 2017: Poewe W, Seppi K, Tanner C M, Halliday G M, Brundin P, Volkmann J, Schrag A E, Lang A E. “Parkinson disease”. Nat Rev Dis Primers 2017, Mar. 23; 3:17013.
Prusiner et al. 2015: Prusiner S B Woerman A L, Mordes D A, WaTDDS J C, Rampersaud R, Berry D B, Patel S², Oehler A, Lowe J K, Kravitz S N, Geschwind D H, Glidden D V, Halliday G M, Middleton L T, Gentleman S M, Grinberg L T, Giles K. “Evidence for α-synuclein prions causing multiple system atrophy in humans with parkinsonism”. Proc Natl Acad Sci USA 2015, Sep. 22; 112(38):E5308-17.
Reich and Grill 2009: Reich S G, Grill S E. “Corticobasal degeneration” Curr Treat Options Neurol. 2009 May; 11(3):179-85.
Samuels et al. 2007: Samuels E R, Hou R H, Langley R W, Szabadi E, Bradshaw C M. “Comparison of pramipexole with and without domperidone co-administration on alertness, autonomic, and endocrine functions in healthy volunteers”. Br J Clin Pharmacol. 2007 November; 64(5):591-602.
Schneider and Mierau 1987: Schneider C S, Mierau J “Dopamine autoreceptor agonists: resolution and pharmacological activity of 2,6-diaminotetrahydrobenzothiazole and an aminothiazole analogue of apomorphine”. J Med Chem 1987, March; 30(3):494-8.
Segawa et al 1976: Segawa M, Hosaka A, Miyagawa F, Nomura Y, Imai H. “Hereditary progressive dystonia with marked diurnal fluctuation”. Adv Neurol. 1976; 14:215-33.
Sengupta et al. 2016: Sengupta U, Nilson A, Kayed R. “The Role of Amyloid-β Oligomers in Toxicity, Propagation, and Immunotherapy”. EBioMedicine 2016, 6, Apr. DOI: 10.1016/j.ebiom.2016.03.035.
Sengupta et al. 2017: Sengupta U, Portelius E, Hansson O, Farmer K, Castillo-Carranza D, Woltjer R, Zetterberg H, Galasko D, Blennow K, Kayed. “Tau oligomers in cerebrospinal fluid in Alzheimer's disease”. Ann Clin Transl Neurol 2017, April; 4(4): 226-235.
Shi et al. 2014: Shi M, Liu C, Cook T J, Bullock K M, Zhao Y, Ginghina C, Li Y, Aro P, Dator R, He C, Hipp M J, Zabetian C P, Peskind E R, Hu S C, Quinn J F, Galasko D R, Banks W A, Zhang J; “Plasma exosomal α-synuclein is likely CNS-derived and increased in Parkinson's disease”. Acta Neuropathol 2014, November; 128(5):639-50. doi: 10.1007/s00401-014-1314-y. Epub 2014 Jul. 6.
Shiyi et al. 2017: Shiyi G, Huang J Jiang H, Han C, Li J, Xu X, Zhang G, Lin Z, Xiong N, Wang T. “Restless Legs Syndrome: From Pathophysiology to Clinical Diagnosis and Management.” Frontiers in Aging Neuroscience 9 (1): 171. doi:10.3389/fnagi.2017.00171.
Soria et al. 2017: Soria F N, Engeln M, Martinez-Vicente M, Glangetas C, López-González M J, Dovero S, Dehay B, Normand E, Vila M, Favereaux A, Georges F, Lo Bianco C, Bezard E, Fernagut P O. “Glucocerebrosidase deficiency in dopaminergic neurons induces microglial activation without neurodegeneration”. Human Molecular Genetics. PMID 28520872 DOI: 10.1093/hmg/ddx120.
Stuendl et al. 2016: Stuendl A, Kunadt M, Kruse N, Bartels C, Moebius W, Danzer K M, Mollenhauer B, Schneider A; “Induction of alpha-synuclein in aggregate formation by CSF exosomes from patients with Parkinson's disease and dementia with Lewy bodies”. Brain 2016, 139; 481-494.
Sweeney et al. 2017: Sweeney P, Park H, Baumann M, Dunlop J, Frydman J, Kopito R, McCampbell A, Leblanc G, Venkateswaran A, Nurmi A, Hodgson R. “Protein misfolding in neurodegenerative diseases: implications and strategies”. Transl Neurodegener 2017, Mar. 13; 6:6. doi: 10.1186/s40035-017-0077-5.
Visanji et al. 2016: Visanji N P, Brotchie J M, Kalia L V, Koprich J B, Tandon A, WaTDDS J C, Lang A E. “α-Synuclein-Based Animal Models of Parkinson's Disease: Challenges and Opportunities in a New Era”. Trends Neurosci 2016, November; 39(11):750-762.

1. A pharmaceutical composition for use for the treatment of a protein misfolding neurodegenerative disease (PMND) in a patient, which comprises a pharmaceutical carrier or vehicle and a fixed-dose combination of a dopamine-agonist Adverse Effects (or Events) Inhibitor (AEsI) selected from the group consisting of domperidone and pharmaceutically acceptable salts and solvates thereof, in an amount equivalent to from 2 mg to 120 mg of domperidone base, and a dopamine agonist selected from the group consisting of pramipexole and pharmaceutically acceptable salts and solvates thereof, in an amount equivalent to from 0.125 mg to 45 mg of pramipexole dihydrochloride monohydrate.

2. The composition of claim 1, wherein said AEsI is selected from the group consisting of domperidone and pharmaceutically acceptable salts and solvates thereof, in an amount equivalent to from 2 mg to 40 mg of domperidone base.

3. The composition according to claim 1, wherein said AEsI is selected from the group consisting of domperidone base, domperidone maleate and domperidone succinate (1:1).

4. The composition according to claim 1, wherein said dopamine agonist is pramipexole or a pharmaceutically acceptable salt thereof, in an amount equivalent to from 15 mg to 25 mg of pramipexole dihydrochloride monohydrate.

5. The composition of claim 4, wherein said pramipexole or pharmaceutically acceptable salt thereof is in an amount equivalent to from more than 20 mg to 25 mg of pramipexole dihydrochloride monohydrate.

6. The composition according to claim 1, wherein said disease is a Parkinson's disease.

7. The composition according to claim 1, wherein said disease is a Parkinson's disease related disorder selected from the group consisting of Restless Leg Syndrome, Dopamine-Responsive Dystonia Syndrome, Progressive Supranuclear Palsy, Fronto-temporal Lobe Dementia, and Corticobasal Degeneration.

8. The composition of claim 1, wherein said domperidone or pharmaceutically acceptable salt or solvate thereof is present in an amount equivalent to from 2 mg to 40 mg of domperidone base; said pramipexole or pharmaceutically acceptable salt or solvate thereof is present in an amount equivalent to from 0.125 mg to 6 mg of pramipexole dihydrochloride monohydrate; and said disease is Restless Leg Syndrome.

9. The composition of claim 8, wherein said pramipexole or pharmaceutically acceptable salt or solvate thereof is present in an amount equivalent to from 0.125 mg to 1 mg of pramipexole dihydrochloride monohydrate.

10. Use of a dopamine-agonist Adverse Effects (or Events) Inhibitor (AEsI) selected from the group consisting of domperidone and pharmaceutically acceptable salts and solvates thereof for the preparation of a medicament for the treatment of a protein misfolding neurodegenerative disease in a fixed-dose combination with pramipexole or a pharmaceutically acceptable salt thereof.

11. The use of claim 10, wherein, in said fixed-dose combination, domperidone or pharmaceutically acceptable salt or solvate thereof is present in an amount equivalent to from 2 mg to 120 mg of domperidone base, and said pramipexole or pharmaceutically acceptable salt or solvate thereof is present in an amount equivalent to from 0.125 mg to 45 mg of pramipexole dihydrochloride monohydrate.

12. The use of claim 10, wherein, in said fixed-dose combination, domperidone or pharmaceutically acceptable salt or solvate thereof is present in an amount equivalent to from 2 mg to 120 mg of domperidone base, and said pramipexole or pharmaceutically acceptable salt or solvate thereof is present in an amount equivalent to from 0.125 mg to 45 mg of pramipexole dihydrochloride monohydrate.

13. A method for treating a disease selected from the group consisting of Parkinson's disease and Parkinson's disease-related disorders in a patient, which comprises administering to said patient at least one dopamine-agonist Adverse Effects (or Events) Inhibitor (AEsI) selected from the group consisting of peripheral DA-antagonists, 5HT3-antagonists, and NK-1 antagonists, in combination with a therapeutically effective daily dose of a dopamine agonist.

14. The method of claim 13, wherein said disease is Parkinson's disease, said AEsI is a peripheral DA-antagonist selected from the group consisting of domperidone and pharmaceutically acceptable salts and solvates and prodrugs thereof, at an effective daily dose equivalent to from 4 mg to 120 mg of domperidone base and said dopamine agonist is selected from the group consisting of pramipexole and pharmaceutically acceptable salt and solvates thereof, at an effective daily dose of from 0.125 mg to 45 mg of pramipexole dihydrochloride monohydrate.

15. The method of claim 14, wherein said AEsI is selected from the group consisting of domperidone and pharmaceutically acceptable salts and solvates thereof, at an effective daily dose equivalent to from 4 mg to 120 mg of domperidone base; and said dopamine agonist is selected from the group consisting of pramipexole and pharmaceutically acceptable salt and solvates thereof, in an effective daily dose equivalent to from more than 4.5 mg to 45 mg of pramipexole dihydrochloride monohydrate.

16. The method of claim 14, wherein said AEsI and said dopamine agonist are each formulated in a pharmaceutical composition in dosage unit form, each in admixture with a pharmaceutical carrier or vehicle.

17. The method of claim 16, wherein said AEsI is selected from group consisting of domperidone and pharmaceutically acceptable salt and solvates thereof, in an amount per unit form equivalent to from 2 mg to 120 mg of domperidone base; and said dopamine agonist is pramipexole or a pharmaceutically acceptable salt thereof, in an amount per unit form equivalent to from 0.125 mg to 45 mg of pramipexole dihydrochloride monohydrate.

18. The method of claim 14, wherein said AEsI and said dopamine agonist are both formulated, as a fixed-dose combination, in a pharmaceutical composition comprising, as an active ingredient, domperidone Component (a), in an amount equivalent to from 2 mg to 120 mg of domperidone base; and, as a second active ingredient, pramipexole Component (b), in an amount equivalent to from 0.125 mg to 45 mg of pramipexole dihydrochloride monohydrate, in admixture with a pharmaceutical carrier or vehicle.

19. A fixed-dose combination consisting of a pharmaceutical composition comprising, as an active ingredient, domperidone Component (a), in an amount equivalent to from 2 mg to 120 mg of domperidone base; and, as a second active ingredient, pramipexole Component (b), in an amount equivalent to from 0.125 mg to 45 mg of pramipexole dihydrochloride monohydrate, in admixture with a pharmaceutical carrier or vehicle.

20. The use of a dopamine adverse effects inhibitor (AEsI) selected from the group consisting of domperidone and pharmaceutically acceptable salts and solvates thereof for the preparation of a medicament for the treatment of a disease selected from the group consisting of Parkinson's disease and Parkinson's disease-related disorders, in combination with a dopamine agonist.

21. A dopamine adverse effects inhibitor (AEsI) selected from the group consisting of domperidone and pharmaceutically acceptable salts and solvates thereof for use for the treatment of a disease selected from the group consisting of Parkinson's disease and Parkinson's disease-related disorders, in combination with a dopamine agonist.

22. A kit comprising the fixed-dose combination of claim 19, and instructions for treatment of a Parkinson's disease or Parkinson's disease-related disorder.
